(19) United States
(12) Patent Application Publication
(10) Pub. No.: US 2026/0263465 A1
(43) Pub. Date: Sep. 10, 2026

(54) REMIBRUTINIB DRUG SUBSTANCE AND DRUG PRODUCT SUBSTANTIALLY FREE OF EPOXIDE IMPURITY

(71) Applicant: Novartis AG, Basel (CH)

(72) Inventors: André BITTERLICH, Weil am Rhein (DE); Gustavo FIGOLI, Riehen (CH); Robert Raymond HUG, Bottmingen (CH); Jasmin MUMINOVIC, Moehlin (CH); Supriya Ramakant RANE, Basel (CH); Karin RAPP, Lörrach (DE); Gina ROEPPISCHER, Basel (CH)

(21) Appl. No.: 19/555,989

(22) Filed: Mar. 4, 2026

Related U.S. Application Data
(60) Provisional application No. 63/767,639, filed on Mar. 6, 2025.
(60) Provisional application No. 63/973,845, filed on Feb. 2, 2026.

(51) Int. Cl. A61K 31/506; A61K 9/16

(52) U.S. Cl. CPC A61K 31/506; A61K 9/1617; A61K 9/1623; A61K 9/1635; A61K 9/1652; A61K 9/1682

(57) ABSTRACT

This invention relates to compositions comprising i)N-(3-(6-Amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, and ii) pharmaceutically acceptable excipients, wherein said composition is substantially free of epoxide impurities, e.g. substantially free of N-(2-((4-cyclopropyl-2-fluorobenzamido)-5-fluoro-2-methylphenyl)-pyrimidin-5-yl)-oxy)ethyl)-N-methyloxirane-2-carboxamide. The invention further relates to novel processes of preparation of such compositions. Pharmaceutical products containing said compositions are also disclosed, as well as methods testing and validating such products for distribution.

### FIELD OF THE INVENTION

The present invention relates to compositions comprising i)N-(3-(6-Amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, and ii) pharmaceutically acceptable excipients, wherein said composition is substantially free of epoxide impurities, e.g. substantially free of N-(2-((4-cyclopropyl-2-fluorobenzamido)-5-fluoro-2-methylphenyl)-pyrimidin-5-yl)-oxy)ethyl)-N-methyloxirane-2-carboxamide.

The invention further relates to novel method of preparation of such compositions. Pharmaceutical products containing said compositions are also disclosed, as well as methods testing and validating such products for distribution.

### BACKGROUND

Epoxides are reactive organic compounds with a three-membered cyclic ether structure. Their triangular structure is highly strain so that they are usually very reactive with a variety of nucleophile. Their high reactivity can present dangers, particularly in biological systems leading to mutagenicity, carcinogenicity, genotoxicity and/or other adverse effects. Epoxides can form in medicines and pharmaceutical products during manufacturing, storage or even use. As a result, their presence is tightly regulated.

To ensure patient safety, regulatory authorities, such as the Food and Drug Administration (FDA) in the United States and the European Medicines Agency (EMA), impose strict limits on the amount of epoxides allowed in pharmaceutical products. Drug manufacturers must implement rigorous quality controls to minimize the formation of these unwanted compounds and ensure compliance with established safety standards. Continuous monitoring of the quality of medicines is therefore crucial to prevent any potential risks to patient health.

N-(3-(6-Amino-5-(2-(N-methylacrylamido)ethoxy)pyrimidin-4-yl)-5-fluoro-2-methylphenyl)-4-cyclopropyl-2-fluorobenzamide, (IUPAC name: N-[3-(6-Amino-5-{2-[methyl(prop-2-enoyl)amino]ethoxy}pyrimidin-4-yl)-5-fluoro-2-methylphenyl]-4-cyclopropyl-2-fluorobenzamide), also known as remibrutinib, is a highly potent and selective oral Bruton's tyrosine kinase (BTK) inhibitor:

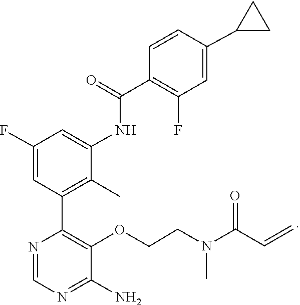 [SMILES: C=CC(=O)N(C)CCOc1c(N)ncnc1-c1cc(F)cc(NC(=O)c2ccc(C3CC3)cc2F)c1C]

Remibrutinib (also known as “LOU064”) was first disclosed in WO2015/079417, filed Nov. 28, 2014, in Example 6. WO2015/079417 is incorporated by reference in its entirety. “LOU064” and “remibrutinib” will be used interchangeably.

Pharmaceutical composition (drug product) comprising remibrutinib, as well as manufacturing process of such composition have been described in WO/2022/162513, filed on Jan. 24, 2022 (attorney docket number PAT059011).

It has now been discovered that the manufacturing process disclosed in WO/2022/162513 provides remibrutinib drug product with the presence of an epoxide impurity at levels which are higher than the level authorized by health authorities for commercial batches. The epoxide impurity is formed by epoxidation of the acrylamide reactive moiety present in remibrutinib and is therefore a degradation product. The epoxide impurity is N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide and has the following structure:

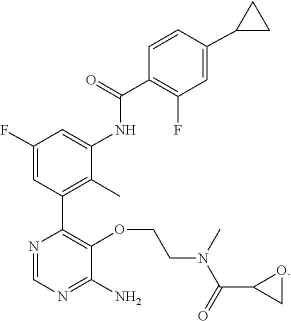 [SMILES: Cc1c(NC(=O)c2ccc(C3CC3)cc2F)cc(F)cc1-c1ncnc(N)c1OCCN(C)C(=O)C1CO1]

It has now been observed that the amount of the epoxide impurity increases during drug product processing and intermediate storage.

It has further been discovered that N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide is positive in an enhanced Ames Test (EAT) and is therefore considered genotoxic by health authorities. Even though the in vivo relevance of such positive EAT has not yet been established, it would be preferable to provide a drug product free or substantially free of a epoxide impurity which provides a positive EAT result, e.g. free of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide. The presence of a epoxide impurity in remibrutinib drug product and its genotoxicity in EAT is first reported in the present application.

It is therefore the object of this invention to provide remibrutinib drug product substantially free of epoxide impurities, e.g. substantially free of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide.

### FIGURES

FIG. 1: 1A: Equipment and depiction of wet media milling process with vessel in recirculation mode; 1B: equipment and depiction of wet media milling process with vessels in pendulum mode

FIG. 2: N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide impurity content versus applied specific energy (E_{M})

FIG. 3: N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide impurity net increase versus applied specific energy (E_{M})

FIG. 4: Particle size reduction versus applied specific energy (E_{M})

FIG. 5: Development of particle size xPCS over milling time

FIG. 6: Development of epoxide impurity (N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) over particle size xPCS

FIG. 7: development of epoxide impurity (N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) over milling time

FIG. 8: Development of epoxide impurity (N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) over storing time and temperature of wet media milling

FIG. 9: Development of epoxide impurity (N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) over milling time for different drug substance batches and copovidone supply

FIG. 10: Obtained particle size versus applied specific energy as defined in instant improved process

FIG. 11: Epoxide impurity ((N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) content versus applied specific energy as defined in instant improved process

FIG. 12: N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide impurity net increase versus applied specific energy (E_{M}) as defined in instant improved process

FIG. 13: Particle size xPCS vs passage number of wet media milling in composition comprising antioxidants

FIG. 14: Polydiversity index vs. passage number of wet medial milling in composition comprising antioxidants

FIG. 15: Epoxide impurity ((N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) content vs. passage number of wet media milling in composition comprising antioxidants.

FIG. 16: Pendulum operation vs. circulation operation and effect on Epoxide impurity ((N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) content

FIG. 17: Evolution of Epoxide impurity ((N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) content during granulation, sieving, tableting and coating

### SUMMARY OF THE INVENTION

The invention relates to reducing the amount of epoxide impurities, e.g. epoxide impurities which are positive in an EAT, e.g. reducing the amount of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide (thereafter REI (remibrutinib epoxide impurity) in the LOU064 drug product to certain levels, for example, where the content of epoxide in LOU064 drug product is no more than 75 ppm, or no more than 50 ppm, or no more than 30 ppm; or no more than 7.5 ppm.

All of these epoxide impurity levels are specified relative to the total amount of the LOU064 in the drug product.

In another aspect, the invention relates to remibrutinib drug product that is substantially free from an impurity that produces a positive EAT, e.g., substantially free from an epoxide impurity; e.g. the epoxide impurity N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide.

In another aspect, the invention relates to remibrutinib drug product, wherein remibrutinib drug product comply with EMA and FDA safety or pharmaceutical product regulations (e,g. new EMA guidance on epoxide impurities, e.g. ICH M7 guidance).

In one aspect, the invention provides a composition comprising or consisting of remibrutinib, or a pharmaceutically acceptable salt of remibrutinib, wherein said composition is substantially free of epoxide impurities, e.g. substantially free of the epoxide impurity of Formula:

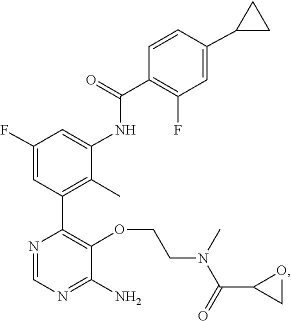 [SMILES: Cc1c(NC(=O)c2ccc(C3CC3)cc2F)cc(F)cc1-c1ncnc(N)c1OCCN(C)C(=O)C1CO1]

having the chemical name N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide (REI). In one aspect, the composition further comprises one or more pharmaceutically acceptable excipients, e.g. one or more excipients selected from surfactant, binder, filler, disintegrant, lubricant and antioxidant as defined herein.

In yet another aspect of the above embodiments, the amount of epoxide, e.g. N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide, in the composition is no more than 75 ppm, e.g. no more than 50 ppm, e.g. no more than 30 ppm, e.g. no more than 7.5 ppm, relative to the total amount of remibrutinib in free or salt form.

In yet another aspect of the above embodiments, the amount of epoxide, e.g. N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide, in the composition is between about 5 ppm and about 75 ppm; or between about 5 ppm and about 30 ppm, e.g. between about 5 ppm and about 7.5 ppm; all relative to the total amount of LOU064 in free form or in a salt form.

In other embodiment, the invention provides method of manufacturing the pharmaceutical composition, comprising i) remibrutinib, or a pharmaceutical acceptable salt thereof, (ii) one or more pharmaceutically acceptable excipients, wherein the pharmaceutical composition is substantially free of epoxide, particularly substantially free of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide (REI). In one aspect of this embodiment, the one or more pharmaceutically acceptable excipients are selected from surfactant, binder, filler, disintegrant, lubricant and antioxidant as defined herein.

The invention also provides pharmaceutical products comprising said compositions and documentation, e.g., in the form of packaging or a package insert. For example, a pharmaceutical product may include a document providing instructions to a patient as to how to administer the composition and/or a document which certifies that the composition is substantially free of epoxide, or at least substantially free of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide (REI).

The invention also provides method of validating remibrutinib drug product for commercial use.

These pharmaceutical compositions are suitable for the treatment of BTK related diseases as disclosed herein.

Further aspects, advantageous features and preferred embodiments of the present invention which are summarized in the following embodiments E1-E91, respectively alone or in combination, contribute to solving the object of the invention:

### Embodiments

E1. A composition comprising or consisting of remibrutinib, or a pharmaceutically acceptable salt of remibrutinib, wherein said composition is substantially free of the epoxide impurity of Formula:

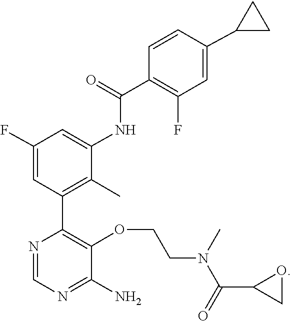 [SMILES: Cc1c(NC(=O)c2ccc(C3CC3)cc2F)cc(F)cc1-c1ncnc(N)c1OCCN(C)C(=O)C1CO1]

E2. A composition of embodiment 1 comprising or consisting of remibrutinib or a pharmaceutically acceptable salt thereof, wherein the total amount of epoxide impurity in the composition is no more than about 75 ppm, e.g. no more than about 50 ppm, e.g. no more than about 30 ppm; or e.g. no more than about 7.5 ppm, relative to the total amount of remibrutinib in free or salt form.
E3. A composition of embodiment 1 comprising or consisting of remibrutinib or a pharmaceutically acceptable salt thereof, wherein the total amount of the epoxide impurity in the composition is no more than the maximum amount of epoxide that is permitted in such a composition, by a regulatory authority, at the time when the composition is prepared and/or administered.
E4. A composition of embodiment 1 comprising or consisting of remibrutinib or a pharmaceutically acceptable salt thereof which has been tested and found to have a total amount of epoxides of no more than 30 ppm, relative to the total amount of remibrutinib in free or salt form.
E5. A composition of embodiment 1 comprising or consisting of remibrutinib or a pharmaceutically acceptable salt thereof which has been tested and found to have a total amount of epoxide impurity that is no more than the maximum amount of epoxides that is permitted in such a composition, by a regulatory authority, at the time when the composition is prepared and/or administered.
E6. A composition according to any one of embodiments 1 to 5, further comprising one or more pharmaceutically acceptable excipients.
E7. A composition according to embodiment 6 wherein one or more pharmaceutically acceptable excipients are selected from surfactant, binder, filler, disintegrant, lubricant and antioxidant.
E8. A pharmaceutical product comprising the composition of any one of embodiments 1 to 7 and instructions to a patient as to how to administer the composition.
E9. A pharmaceutical product containing: (a) a composition comprising remibrutinib or a pharmaceutically acceptable salt thereof according to any one of embodiments 1 to 7; and (b) a document which certifies, either directly or via a link to an electronic database, that the total amount of epoxide in the composition is no more than 30 ppm, relative to the total amount of remibrutinib in free or salt form.
E10. A pharmaceutical product containing: (a) a composition comprising remibrutinib or a pharmaceutically acceptable salt thereof according to any one of embodiments 1 to 7; and (b) a document which certifies, either directly or via a link to an electronic database, that the total amount of epoxide in the composition is no more than the maximum amount of epoxide that is permitted in such a composition, by a regulatory authority, at the time when the composition is prepared and/or administered.
E11. The pharmaceutical product of any one of embodiments 8 to 10, wherein the product further comprises instructions to a patient as to how to administer the composition.
E12. The composition or pharmaceutical product of any one of embodiments 1 to 11, wherein the total amount of remibrutinib or a pharmaceutically acceptable salt thereof in the composition provides the same amount of remibrutinib as 10 mg, 25 mg or 100 mg of remibrutinib free base. E13. A composition or pharmaceutical product according to any one of embodiments 1 to 12, wherein the composition is an oral dosage form, optionally wherein the oral dosage form is a tablet.
E14. A composition or pharmaceutical product according to any one of embodiments 1 to 12, wherein the composition consists of remibrutinib or a pharmaceutically acceptable salt thereof within an orally administrable capsule or tablet.
E15. A composition or pharmaceutical product according to any one of embodiments 1 to 14, wherein the composition is present in a sealed container together with desiccant.
E16. A method of preparing a composition comprising remibrutinib or a pharmaceutically acceptable salt of remibrutinib and one or more pharmaceutically acceptable excipients, wherein the method comprises a step of particle size reduction of remibrutinib by a wet media milling process wherein the wet media milling process is performed with a specific energy between 600 and 1000 KJ/Kg, preferably between 600 and 800 KJ/Kg.
E17. A method of preparing a composition comprising remibrutinib or a pharmaceutically acceptable salt of remibrutinib and one or more pharmaceutically acceptable excipients according to embodiment 16, wherein the wet media milling process is performed at an average stress energy process parameter of no more than 1.3 pNm.
E18. The method of preparing a composition comprising remibrutinib or a pharmaceutically acceptable salt of remibrutinib and one or more pharmaceutically acceptable excipients according to embodiment 16 or 17, wherein the wet media milling process is performed in grinding chamber with suspension outlet temperature of no more than 20° C.
E19. The method of preparing a composition comprising i) remibrutinib or a pharmaceutically acceptable salt of remibrutinib and ii) one or more pharmaceutically acceptable excipients according to any one of embodiments 16 to 18, wherein the wet media milling process is performed in recirculation mode with a turnover number of between about 28 to about 106.
E20. The method of preparing a composition comprising i) remibrutinib or a pharmaceutically acceptable salt of remibrutinib and ii) one or more pharmaceutically acceptable excipients according to any one of embodiments 16 to 19, wherein the wet media milling process generates a wet media milled suspension with a particle size of remibrutinib of less or equal to 200 nm.
E21. The method of preparing a composition comprising i) remibrutinib or a pharmaceutically acceptable salt of remibrutinib and ii) one or more pharmaceutically acceptable excipients according to any one of embodiments 16 to 20, wherein the wet media milling process is performed in the presence of one or more antioxidants.
E22. The method of embodiment 21, wherein the one or more antioxidant is selected from Thymol, Propyl galate, Citric acid, Tartaric acid, ascorbic acid, Sodium ascorbate, gentisic acid, Mequinol, Butylated hydroxy toluene, butylated hydroxy anisole, sodium metabisulfite or combination thereof, preferably Butylated hydroxy toluene, butylated hydroxy anisole, ascorbic acid, Sodium ascorbate, gentisic acid, sodium metabisulfite or combination thereof, or more preferably ascorbic acid, Sodium ascorbate, gentisic acid or combination thereof.
E23. The method of embodiments 21 or 22, wherein the antioxidant or mixture thereof are present in the wet media milling suspension in a total antioxidant amount of about 0.01% to about 0.5% weight by weight of the wet media milled suspension, preferably 0.01% to 0.2% w/w.
E24. The method of preparing a composition comprising i) remibrutinib or a pharmaceutically acceptable salt of remibrutinib and ii) one or more pharmaceutically acceptable excipients according to any one of embodiments 16 to 23, further comprising additional steps of preparing suspension for a spray granulation step.
E25. The method of embodiment 24 wherein the suspension for spray granulation comprises one or more antioxidant selected from Thymol, Propyl galate, Citric acid, Tartaric acid, ascorbic acid, Sodium ascorbate, gentisic acid, Mequinol, Butylated hydroxy toluene, butylated hydroxy anisole, sodium metabisulfite or combination thereof, preferably Butylated hydroxy toluene, butylated hydroxy anisole, ascorbic acid, Sodium ascorbate, gentisic acid, sodium metabisulfite or combination thereof, or more preferably ascorbic acid, Sodium ascorbate, gentisic acid or combination thereof.
E26. The method of embodiment 25 wherein the one or more antioxidant or mixture thereof are present in the suspension for spray granulation in a total antioxidant amount of about 0.01% to about 0.5% weight by weight of the suspension for spray granulation, preferably 0.01% to 0.2% w/w.
E27. The method of any of embodiments 20 to 26, wherein the media milled suspension and the suspension for spray granulation comprises one or more pharmaceutically acceptable excipients selected from a surfactant and a binder.
E28. The method of embodiment 27, wherein the media milled suspension and the suspension for spray granulation comprises a surfactant selected from sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof, preferably the surfactant is sodium lauryl sulfate.
E29. The method of embodiment 27 wherein the media milled suspension and the suspension of spray granulation comprises a binder selected from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, vitamin E TPGS, or a mixture thereof, preferably the binder is polyvinylpyrrolidone-vinyl acetate copolymer (copovidone).
E30. The method of any one of embodiments 24 to 29, further comprising the step of spray granulation wherein the suspension for spray granulation is sprayed onto an inert substrate to provide a granule.
E31. The method of embodiment 30 wherein the spray granulation is performed with a spray rate at steady state of between 500 and 600 g/min.
E32. The method of embodiment 30 or 31, wherein the spray granulation step is performed with an atomization air pressure of between about 2.5 and about 3.5 bar.
E33. The method of any one of embodiments 30 to 32 wherein the inert substrate is selected from the group consisting of lactose, microcrystalline cellulose, mannitol, sucrose, starch, granulated hydrophilic fumed silica, or mixtures thereof, preferably the material, which is selected from the group consisting of lactose, mannitol, or mixtures thereof and most preferably the material is mannitol.
E34. The method of any one of embodiments 16 to 33, further comprising the step of preparing a final blend.
E35. The method of embodiment 34, wherein the final blend comprises one or more pharmaceutically acceptable excipient is selected from a filler, a disintegrating agent, a lubricating agent, and a gliding agent.
E36. The method of embodiment 35, wherein the filler is selected from calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof preferably mannitol or cellulose or mixture thereof E37. The method of embodiment 35 or 36 wherein the disintegrating agent is selected from croscarmellose sodium, crospovidone, sodium starch glycolate, corn starch, or alginic acid, or mixture thereof.
E38. The method of any one of embodiment 35-37 wherein the lubricating agent is selected from magnesium stearate, sodium stearyl fumarate, stearic acid or talc or mixture thereof.
E39. The method according to any one of embodiment 35-38, further comprising step of compressing the final blend into orally administrable dosage form, optionally a tablet.
E40. The method according to any one of embodiments 35 to 39 wherein the pharmaceutically acceptable excipients have a peroxide content of less than 30 ppm, less than 20 ppm or less than 10 ppm, e.g. copovidone as a binder has a peroxide content of less than 30 ppm, less than 20 ppm or less than 10 ppm.
E41. A method of evaluating a composition comprising or consisting of remibrutinib or a pharmaceutically acceptable salt thereof, the method comprising testing the composition for the presence or amount of epoxide of Formula:

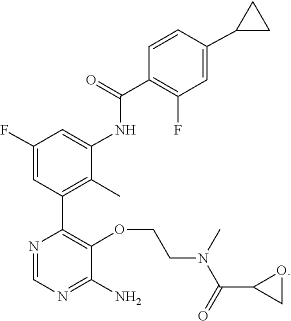 [SMILES: Cc1c(NC(=O)c2ccc(C3CC3)cc2F)cc(F)cc1-c1ncnc(N)c1OCCN(C)C(=O)C1CO1]

E42. A method of validating a process for the production of a composition comprising or consisting of remibrutinib or a pharmaceutically acceptable salt thereof, the method comprising testing the composition produced by the method according to any one of embodiments 15 to 34, for the presence or amount of epoxide of Formula:

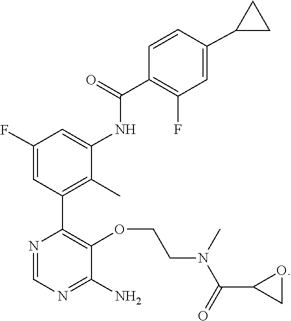 [SMILES: Cc1c(NC(=O)c2ccc(C3CC3)cc2F)cc(F)cc1-c1ncnc(N)c1OCCN(C)C(=O)C1CO1]

E43. A method of obtaining regulatory approval for a pharmaceutical composition which comprises or consists of remibrutinib or a pharmaceutically acceptable salt thereof, wherein the method comprises testing a sample of the composition for the presence or amount of remibrutinib epoxide impurity (REI) of Formula:

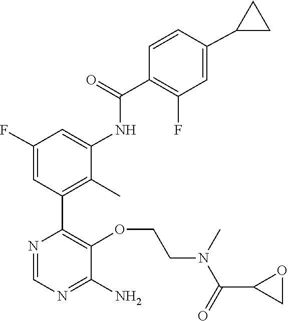 [SMILES: Cc1c(NC(=O)c2ccc(C3CC3)cc2F)cc(F)cc1-c1ncnc(N)c1OCCN(C)C(=O)C1CO1]

and submitting the results of said testing to a regulatory authority.
E44. The method of any one of embodiments 41 to 43, wherein a batch of the composition is tested.
E45. A process for preparing a pharmaceutical product comprising a composition that comprises remibrutinib or a pharmaceutically acceptable salt thereof, and one or more pharmaceutically acceptable excipients, the process comprising:
   a. obtaining a batch of a wet media milled suspension comprising remibrutinib or a pharmaceutically acceptable salt thereof;
   b. determining the total amount of remibrutinib epoxide impurity (REI) in said batch; and
   c. preparing the pharmaceutical product from the batch only if the batch of wet medial milled suspension is determined to have a total amount of epoxide of no more than 30 ppm, relative to the total amount of remibrutinib in free or salt form.
E46. A process of distributing a validated batch of a pharmaceutical product comprising remibrutinib or a pharmaceutically acceptable salt thereof and one or more pharmaceutically acceptable excipients, the process comprising:
   a. producing a batch of the pharmaceutical product;
   b. performing stability testing with a sample of the batch;
   c. determining the total amount of remibrutinib epoxide impurity of Formula:

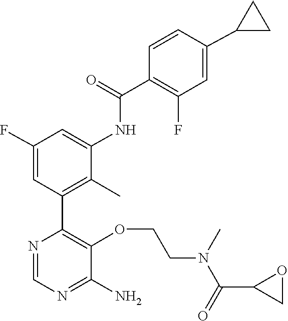 [SMILES: Cc1c(NC(=O)c2ccc(C3CC3)cc2F)cc(F)cc1-c1ncnc(N)c1OCCN(C)C(=O)C1CO1]

in said batch after stability testing; and
d. validating the batch for distribution only if the sample of the batch after stability testing is determined to have a total amount of epoxide of no more than 75 ppm, no more than 30 ppm or no more than 7.5 ppm, relative to the total amount of remibrutinib in free or salt form.
E47. The process of any one of embodiments 41 to 46, wherein the step of testing or determining the amount of epoxide is performed using HPLC, e.g. using validation method as described in example 2.
E48. Use of epoxide of Formula:

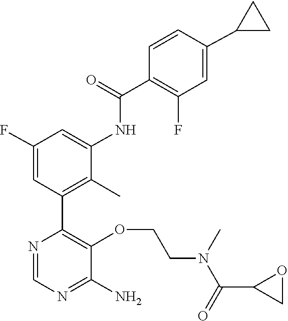 [SMILES: Cc1c(NC(=O)c2ccc(C3CC3)cc2F)cc(F)cc1-c1ncnc(N)c1OCCN(C)C(=O)C1CO1]

as a reference standard to detect an impurity in a composition comprising remibrutinib or a pharmaceutically acceptable salt thereof.
E49. The use of embodiment 48, wherein the impurity is

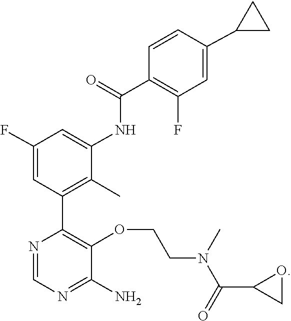 [SMILES: Cc1c(NC(=O)c2ccc(C3CC3)cc2F)cc(F)cc1-c1ncnc(N)c1OCCN(C)C(=O)C1CO1]

E50. The use of embodiment 48 or 49, wherein the detection is performed using described validation method in example 2.
E51. A method of storing a composition comprising or consisting of remibrutinib, wherein the method comprises storing the composition in the presence of a desiccant, optionally wherein the method comprises testing the composition for the presence or amount of remibrutinib epoxide impurity.
E52. A method of storing a composition comprising or consisting of remibrutinib, wherein the method comprises storing the composition at a temperature of less than 25° C.
E53. A method of storing wet media milled suspension and spray granulation suspension, wherein method comprises storing the suspensions at a temperature of less than 25° C., e.g. less than 15° C., less than 10° C., e.g. between 5° C. and 8° C., optionally wherein the method comprises testing the suspensions for the presence or amount of remibrutinib epoxide impurity.
E54. A composition or pharmaceutical product according to any one of embodiments 1 to 15, wherein said composition has a shelf life of at least 1, 2, 3, 4, 5, 6, 7, 8, 9, 10, 11, 12, 13, 14, 15, 16, 17, 18, 24, 30, 36, 42, 48, 54 or 60 months when stored at a temperature of no more than 40° C., preferably not more than 30° C., more preferably no more than 25° C., optionally at a temperature of 20 to 25° C.
E55. The composition or pharmaceutical product according to embodiment 54, wherein said composition has a shelf life of at least 9 months.
E56. The composition or pharmaceutical product according to any one of embodiments 54 or 55, wherein the shelf life is the period of time in which the total amount of remibrutinib epoxide impurity (REI, i.e. compound of Formula (1)), in the composition remains no more than 30 ppm, preferably no more than 15 ppm, or no more than 7.5 ppm, relative to the total amount of remibrutibib in free or salt form.
E57. A pharmaceutical composition for oral administration comprising remibrutinib, an inert substrate, a binder, a surfactant and one or more antioxidants wherein the weight ratio between remibrutinib and the binder is in a range of about [3:1] to about [1;1], e.g. about [3:1], about [2:1] or about [1:1], or about [1:2] or about [1:3], preferably about [1:1] and more preferably about [2:1].
E58. The pharmaceutical composition according to embodiment 57 wherein the inert substrate comprises a material which is selected from the group consisting of lactose, microcrystalline cellulose, mannitol, sucrose, starch, granulated hydrophilic fumed silica, or mixtures thereof, preferably the material, which is selected from the group consisting of lactose, mannitol, or mixtures thereof and most preferably the material is mannitol.
E59. The pharmaceutical composition according to embodiment 57 or 58, wherein the binder is selected from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, or a mixture thereof, preferably the binder is polyvinylpyrrolidone-vinyl acetate copolymer.
E60. The pharmaceutical composition according to any one of embodiments 57-59, wherein the surfactant is selected from the group consisting of sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof, preferably the surfactant is sodium lauryl sulfate.
E61. The pharmaceutical composition according to any one of embodiments 57-60, wherein the one or more antioxidant is selected from Thymol, Propyl galate, Citric acid, Tartaric acid, ascorbic acid, Sodium ascorbate, gentisic acid, Mequinol, Butylated hydroxy toluene, butylated hydroxy anisole, sodium metabisulfite or mixture thereof, preferably Butylated hydroxy toluene, butylated hydroxy anisole, ascorbic acid, Sodium ascorbate, gentisic acid, sodium metabisulfite or mixture thereof, or more preferably ascorbic acid, sodium ascorbate and gentisic acid, or mixture thereof.
E62. The pharmaceutical composition according to embodiment 61 wherein the inert substrate is mannitol, the binder is polyvinylpyrrolidone-vinyl acetate copolymer and the surfactant is sodium lauryl sulfate.
E63. The pharmaceutical composition according to any embodiments 57 to 62 wherein the antioxidant is gentisic acid.
E64. The pharmaceutical composition according to any one of embodiments 57 to 63, further comprising one or more pharmaceutically acceptable excipients selected from a filler, a disintegrating agent and a lubricating agent.
E65. The pharmaceutical composition of embodiment 64 wherein the composition comprises one or more fillers selected from calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof preferably mannitol or cellulose or mixture thereof.
E66. The pharmaceutical composition of embodiment 64 or 65, wherein the composition comprises one or more disintegrating agent selected from croscarmellose sodium, crospovidone, sodium starch glycolate, corn starch, or alginic acid, or mixture thereof.
E67. The pharmaceutical composition according to any one of embodiments 64-66 wherein the composition comprises one or more lubricating agent selected from magnesium stearate, sodium stearyl fumarate, stearic acid or talc or mixture thereof.
E68. The pharmaceutical composition according to any one of embodiments 64-67 wherein the composition comprises mannitol and cellulose as fillers, sodium stearyl fumarate or magnesium stearate as lubricant, and croscamellose sodium or sodium carbonate as disintegrating agent.
E69. The pharmaceutical composition according to any one of embodiments 64-68, wherein remibrutinib is present in an amount of about 10% w/w to about 25% w/w, preferably about 19% or about 20% based on the total weight of the composition.
E70. The pharmaceutical composition according to any one of embodiments 64 to 69, wherein a filler is present in an amount of about 20 to about 40% w/w based on the total weight of the composition.
E71. The pharmaceutical composition according to any one of embodiments 64 to 70, wherein a disintegrating agent is present in an amount of about 5% w/w to about 10% w/w, preferably about 5 or about 6% based on the total weight of the composition.
E72. The pharmaceutical composition according to any one of embodiments 64 to 71 wherein the inert substrate is present in an amount of about 20% w/w to about 40% w/w, preferably about 30% based on the total weight of the composition.
E73. The pharmaceutical composition according to any one of embodiments 64 to 72 wherein the binder is present in an amount of about 5% w/w to about 25% w/w, preferably about 8 to about 12% w/w, based on the total weight of the composition.
E74. The pharmaceutical composition according to any one of embodiments 64 to 73 wherein a lubricant is present in an amount of about 0.1 to about 2% w/w, preferably about 0.5% w/w to about 1.5% w/w based on the total weight of the composition.
E75. The pharmaceutical composition according to any one of embodiments 64 to 74 wherein a surfactant is present in an amount of about 0.1% w/w to about 2.5% w/w, preferably about 0.2% w/w to about 0.8% w/w based on the total weight of the composition. 76. The pharmaceutical composition according to any one of embodiments 64 to 75 wherein the antioxidant is present in an amount of about 0.01% to about 0.5% w/w, preferably about 0.03% to about 0.1% w/w based on the total weight of the composition.
E77. The pharmaceutical composition according to embodiment 76 wherein the antioxidant is gensitic acid and is present in an amount of about 0.01% to about 0.2% w/w based on the total weight of the composition.
E78. The pharmaceutical composition according to embodiment 76 wherein the antioxidant is ascorbic acid or sodium ascorbate or mixture thereof and the total amount of antioxidant is of about 0.01% to about 0.5% w/w based on the total weight of the composition.
E79. The pharmaceutical composition of embodiment 57 wherein:
   remibrutinib is present in an amount of 19.2% w/w, polyvinylpyrrolidone-vinyl acetate polymer is present in an amount of 9.6% w/w SLS is present in an amount of 0.8% w/w Mannitol substrate is present in an amount of 30.37% w/w Mixture of cellulose and mannitol are present in a total amount of 33% w/w (50:50 each w/w) Croscamellose is present in an amount of 6% w/w Sodium stearyl fumarate is present in an amount of 1% w/w, Gensitic acid is present in an amount of 0.03% w/w, wherein each percentage is expressed by weight of the total weight of the composition.
E80. The pharmaceutical composition of embodiment 57 wherein:
   remibrutinib is present in an amount of 19.2% w/w, polyvinylpyrrolidone-vinyl acetate polymer is present in an amount of 9.6% w/w SLS is present in an amount of 0.8% w/w Mannitol substrate is present in an amount of 30.35% w/w Mixture of cellulose and mannitol are present in a total amount of 33% w/w (50:50 each w/w) Croscamellose is present in an amount of 6% w/w Sodium stearyl fumarate is present in an amount of 1% w/w, Ascorbic acid is present in an amount of 0.05% w/w, wherein each percentage is expressed by weight of the total weight of the composition.
E81. The pharmaceutical composition of embodiment 57 wherein:
   remibrutinib is present in an amount of 19.2% w/w, polyvinylpyrrolidone-vinyl acetate polymer is present in an amount 9.6% w/w SLS is present in an amount of 0.8% w/w Mannitol substrate is present in an amount of 30.3% w/w Mixture of cellulose and mannitol are present in a total amount of 33% w/w (50:50 each w/w) Croscamellose is present in an amount of 6% w/w Sodium stearyl fumarate is present in an amount of 1% w/w, Sodium ascorbate is present in an amount of 0.1% w/w, wherein each percentage is expressed by weight of the total weight of the composition.
E82. The pharmaceutical composition according to any one of embodiments 57-81, wherein the particle size of remibrutinib is less than 200 nm, e.g. the particle size of remibrutinib as measured by PCS is between about 110 nm and about 180 nm.
E83. The pharmaceutical composition according to any one of embodiments 57-82 wherein the content of REI (compound of Formula (1) is less than 30 ppm, less than 15 ppm, less than 10 ppm or less than 7.5 ppm.
E84. A final dosage form which is a tablet formulation comprising the pharmaceutical composition of any one of embodiments 57-83.
E85. The final dosage form according to embodiment 84 wherein the tablet further comprises a coating.
E86. The composition according to any one of embodiments 1 to 7, 12 to 15 and 57-82 wherein the composition was obtained by a method of any one of embodiments 16 to 40.
E87. The composition of any one of embodiments 1 to 7 and 11 to 15 and 54 to 83, or the pharmaceutical product of any one of embodiments 8 to 15 and 54 to 56, for use in the treatment of a diseases or disorder mediated by BTK or ameliorated by inhibition of BTK, e.g. Autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis especially relapsing multiple sclerosis, myasthenia gravis, Sjogren's syndrome, hidradenitis suppurativa, IgE driven allergy, e.g. drug, venom, food allergy; autoimmune hemolytic anemia, anti-neutrophil cytoplasmic antibodies (ANCA)-associated vasculitis, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerulonephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of hematopoietic origin including, but not limited to, multiple myeloma; a leukemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenström disease in a patient in need of such treatment.
E88. A method of treating a disease or a disorder mediated by BTK or ameliorated by inhibition of BTK, in a patient in need of such treatment, comprising administering a composition according to any one of embodiments 1 to 7 and 11 to 15 and 54 to 83, or administering a pharmaceutical product according to any one of embodiments 8 to 15 and 54 to 56, wherein the disease or disorder is selected from an autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis especially relapsing multiple sclerosis, myasthenia gravis, Sjogren's syndrome, hidradenitis suppurativa, IgE driven allergy, e.g. drug, venom, food allergy; autoimmune hemolytic anemia, anti-neutrophil cytoplasmic antibodies (ANCA)-associated vasculitis, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerulonephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of hematopoietic origin including, but not limited to, multiple myeloma; a leukemia; acute myelogenous leukemia; chronic myelogenous leukemia; lymphocytic leukemia; myeloid leukemia; non-Hodgkin lymphoma; lymphomas; polycythemia vera; essential thrombocythemia; myelofi-brosis with myeloid metaplasia; and Waldenström disease.
E89. The composition, pharmaceutical product, for use according to embodiment 87, or the method of embodiment 88, wherein the treatment comprises administering to the patient a daily amount of remibrutinib of 20 mg, or a daily amount of the pharmaceutically acceptable salt of remibrutinib which corresponds to 20 mg of remibrutinib free base.
E90. The composition, pharmaceutical product for use according to embodiment 88, or the method of embodiment 87 wherein the treatment comprises administering to the patient a daily amount of remibrutinib of 50 mg, or a daily amount of the pharmaceutically acceptable salt of remibrutinib which corresponds to 50 mg of remibrutinib free base.
E91. The composition, pharmaceutical product for use according to embodiment 88, or the method of embodiment 89, wherein the treatment comprises administering to the patient a daily amount of remibrutinib of 200 mg, or a daily amount of the pharmaceutically acceptable salt of remibrutinib which corresponds to 200 mg of remibrutinib free base.
E92. The composition, pharmaceutical product for use according to embodiment 88, or a method according to embodiment 89 wherein the patient to be treated receives a daily amount of epoxide, e.g. remibrutinib epoxide impurity (REI) of no more than 1.5 ug.
E93. The composition, pharmaceutical product for use according to embodiment 88 or a method according to embodiment 89, wherein remibrutinib is in a crystalline form A as defined herein.
E94. A composition comprising remibrutinib or a pharmaceutically acceptable salt thereof, obtained or obtainable according to the method of any one of embodiments 16 to 40.

### DETAILED DESCRIPTION

### Definition

The term “drug substance” is the substance comprising an active pharmaceutical ingredient which is intended to be used in the manufacture of a drug product (i.e. pharmaceutical composition, e.g. tablet, capsule etc.). The active pharmaceutical ingredient (i.e. LOU064 (remibrutinib)) is present in the drug substance with various degrees of purities. The terms “drug substance”, “LOU064 drug substance” and “remibrutinib drug substance” are used interchangeably. Therefore “LOU064 drug substance” is LOU064 with various degrees of purities. In one embodiment, remibrutinib is substantially pure (e.g. substantially chemically pure) as defined below. In another embodiment, remibrutinib is in a crystalline form as disclosed in WO2020/234779, for example anhydrous crystalline form A as disclosed in example 1 of WO2020/234779. In one aspect of this embodiment, remibrutinib in in the form of crystalline form A and is substantially phase pure. In another embodiment, remibrutinib is both substantially chemically pure and substantially phase pure. In one embodiment, remibrutinib is additionally substantially free of the epoxide impurity, e.g. substantially free of N-(2-((4-cyclopropyl-2-fluorobenzamido)-5-fluoro-2-methylphenyl)-pyrimidin-5-yl)-oxy)ethyl)-N-methyloxirane-2-carboxamide.

Remibrutinib drug substance is manufactured according to processes described in WO/2024/069507(attorney docket number PAT059209-WO-PCT), incorporated herein by reference. Preferably, remibrutinib drug substance is manufactured according to processes described in PCT application Number WO/2025/158336 (attorney docket number PAT059622-WO-PCT, and in PCT application number WO/2025/210469 (attorney docket number PAT059651-WO-PCT), both applications are incorporated herein by reference.

The terms “crystalline form(s)” or “crystalline modification(s)” or “polymorphic form(s)” or “polymorph(s)” will be used interchangeably herein. As used herein “polymorph” refers to crystalline forms having the same chemical composition but different spatial arrangements of the molecules, atoms, and/or ions forming the crystal. Each polymorph differs with respect to thermodynamic stability, physical parameters, x-ray structure and methods of preparation.

As used herein, “substantially pure,” when used in reference to LOU064, means a compound having a purity greater than 90 weight %, including greater than 90, 91, 92, 93, 94, 95, 96, 97, 98, and 99 weight %, and also including equal to about 100 weights % of remibrutinib, based on the weight drug substance.

The remaining material comprises other form(s) of the compound, and/or reaction impurities and/or processing impurities arising from its preparation. For example, a crystalline form of remibrutinib may be deemed substantially pure in that it has a purity greater than 90 weight %, as measured by means that are at this time known and generally accepted in the art, where the remaining less than 10 weight % of material comprises other form(s) of remibrutinib and/or reaction impurities and/or processing impurities.

As used herein, “substantially phase pure,” when used in reference to crystalline form of LOU064, means remibrutinib having a phase purity of greater than about 90% by weight, including greater than about 90, 91, 92, 93, 94, 95, 96, 97, 98, and about 99% by weight, and also including equal to about 100% by weight of the remibrutinib, based on the weight of the LOU064 on anhydrous basis. The term “phase pure” or “phase purity” herein refers to phase homogeneity with respect to a particular solid-state form of remibrutinib and does not necessarily imply a high degree of chemical purity absent an express statement to that effect. Phase purity may be determined according to methods known in the art, for example, using XRPD to do quantitative phase analysis using one or more approaches known in the art, for example, via an external standard method, direct comparisons of line (peak) characteristics which are attributed to different phases in a particular spectra, or via an internal standard method. However, XRPD quantification of phase purity can be complicated by the presence of amorphous material. Accordingly, other methods that may be useful for determining phase purity include, for example, solid state NMR spectroscopy, Raman and/or infrared spectroscopy. A person skilled in the art would readily understand these methods and how to employ these additional (or alternative) methods for determining phase purity.

As used herein, “substantially chemically pure” when used in reference to remibrutinib, means remibrutinib having a chemical purity greater than about 90% by weight, including greater than about 90, 91, 92, 93, 94, 95, 96, 97, 98, and about 99% by weight, and also including equal to about 100% by weight of remibrutinib based on the weight of the drug substance. The remaining material generally comprises other compounds, such as, for example, reaction impurities, starting materials, reagents, side products, and/or other processing impurities arising from the preparation and/or isolation and/or purification of the remibrutinib. For example, remibrutinib may be deemed to be substantially chemically pure if it has been determined to have a chemical purity of greater than about 90% by weight, as measured by standard and generally accepted methods known in the art, where the remaining less than about 10% by weight constitutes other materials such as other stereoisomers of the compound of Formula (1), reaction impurities, starting materials, reagents, side products, and/or processing impurities. Chemical purity may be determined according to methods known in the art, for example, high performance liquid chromatography (HPLC), LC-MS (liquid chromatography—mass spectrometry), nuclear magnetic resonance (NMR) spectroscopy, or infrared spectroscopy. One of skill in the art would readily understand these methods and how to employ these additional (or alternative) methods for determining chemical purity.

In an embodiment, remibrutinib or any other compound described herein may be provided as a salt. As used herein, the terms “salt” or “salts” refers to an acid addition or base addition salt of a compound disclosed herein. “Salts” include in particular “pharmaceutically acceptable salts”. The term “pharmaceutically acceptable salts” refers to salts that retain the biological effectiveness and properties of a compound disclosed herein and, which typically are not biologically or otherwise undesirable. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In some embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and trimethamine. The pharmaceutically acceptable salts of the compounds disclosed herein can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

In some embodiment remibrutinib or a pharmaceutically acceptable salt thereof drug substance is substantially free of the epoxide impurity; e.g. substantially free of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide:

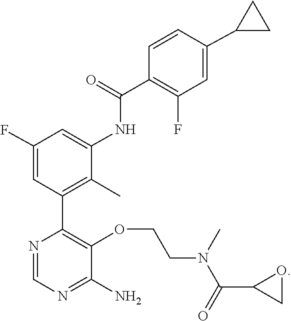 [SMILES: Cc1c(NC(=O)c2ccc(C3CC3)cc2F)cc(F)cc1-c1ncnc(N)c1OCCN(C)C(=O)C1CO1]

In yet another aspect of the above embodiment, the amount of epoxide, e.g. the amount of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide in the drug substance is no more than 30 ppm, e.g. no more 15 ppm, e.g. no more than 7.5 ppm, e.g. no more than 5 ppm or no more than 2 ppm.

The term “remibrutinib drug product” is a pharmaceutical composition comprising i) remibrutinib drug substance and ii) one or more pharmaceutically acceptable excipients, e.g. a capsule or tablet. “Composition” and “pharmaceutical composition” are used herein interchangeably. Remibrutinib drug product: remibrutinib composition substantially free of epoxide impurities, particularly substantially free of N-(2-((4-cyclopropyl-2-fluorobenzamido)-5-fluoro-2-methylphenyl)-pyrimidin-5-yl)-oxy)ethyl)-N-methyloxirane-2-carboxamide; and remibrutinib pharmaceutical product.

The expression “comprise”, as used herein, besides its literal meaning also includes and specifically refers to the expressions “consist essentially of and “consist of. Thus, the expression “comprise” refers to embodiments wherein the subject-matter which “comprises” specifically listed elements may and/or indeed does encompass further elements as well as embodiments wherein the subject-matter which “comprises” specifically listed elements does not comprise further elements.

Numeric ranges described herein are inclusive of the numbers defining the range. The headings provided herein are not limitations of the various aspects or embodiments of this invention which can be read by reference to the specification as a whole. According to one embodiment, subject matter described herein as comprising certain steps in the case of methods or as comprising certain ingredients in the case of compositions refers to subject matter consisting of the respective steps or ingredients. It is preferred to select and combine specific aspects and embodiments described herein and the specific subject-matter arising from a respective combination of specific embodiments also belongs to the present disclosure.

For purposes of interpreting this specification, the following definitions will apply and whenever appropriate, terms used in the singular will also include the plural and vice versa.

The term “substantially free” of epoxide impurity means the content of epoxide (e.g. in the drug product) is no more than about 75 ppm, no more than about 50 ppm, no more than about 30 ppm no more than about 7.5 ppm or no more than about 5 ppm. The term “about” is used to account for detection method variabilities.

The terms “ppm” and “ppb” indicates respectively part per million and part per billion. Those terms represent a weight-to-weight ratio used to describe concentration. For example, part per billion (ppb) of epoxide in the drug substance is the number of units mass of a contaminant (e.g. epoxide impurity (e.g. epoxide N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) per 1000 million units of total mass of drug substance.

Mass of drug substance is the sum of the mass of LOU064 (free base) and the mass of all impurities (including epoxide impurities).

This ppb ratio can be expressed as follow:

$$\frac{Mass of epoxide}{Mass of drug substance} \times {10}^{9}$$

Similarly, a ppm ratio can be expressed as follow:

$$\frac{Mass of epoxide}{Mass of drug substance} \times {10}^{9} .$$

The content of epoxide in ppm (or ppb) in a pharmaceutical composing comprising remibrutinib or a pharmaceutically acceptable salt thereof (i.e. in the drug product), is determined relative to the amount of remibrutinib in free or salt form. (i.e. for a pharmaceutical composition comprising remibrutinib, the content of epoxide in such composition is determined relative to remibrutinib, and for a pharmaceutical composition comprising remibrutinib salt, the content of epoxide is determined relative to the amount of remibrutinib salt).

The content in ppm of peroxide in an excipient represents a weight-by-weight ratio of peroxide versus excipient, i.e. mass of peroxide divided by mass of excipient and multiply by 10⁶.

The amount of epoxide impurity (e.g. N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) is measured using mass spectrometry (MS) coupled with separation methods (e.g. liquid chromatography or high performance liquid chromatography (HPLC, e.g. reversed phase HPLC)). In one embodiment, the detection level sensitivity can be enhanced by using mass spectrometry selection ion monitoring (SIM), wherein the scanning mode is tuned to detect only selected mass of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide. In one embodiment, the determination of the epoxide content (i.e. N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide) in the drug substance or drug product (i.e. in pharmaceutical composition comprising LOU064 or pharmaceutically acceptable salt thereof) is determined according to method described in example 2. One of skill in the art would readily understand these methods and how to employ additional (or alternative) methods for determining amounts of epoxide impurities in low ppm ranges.

The term “about”, as used herein, is intended to provide flexibility to a numerical range endpoint, providing that a given value may be “a little above” or “a little below” the endpoint accounting for variations one might see in the measurements taken among different instruments, samples, and sample preparations. The term usually means within 5%, e.g. within 1% of a given value or range. For example, 50 ppb within 5% means 50 ppm +/−2.5 ppm (between 47.5 ppm and 52.5 ppm) or 50 ppm within 1% means 50 ppm +/−0.5 ppm (i.e. between 49.5 ppm and 50.5 ppm).

In one aspect, the invention provides a composition comprising or consisting of i) remibrutinib, or a pharmaceutically acceptable salt of remibrutinib, wherein said composition is substantially free of epoxide impurity, e.g. substantially free of N-(2-((4-cyclopropyl-2-fluorobenzamido)-5-fluoro-2-methylphenyl)-pyrimidin-5-yl)-oxy)ethyl)-N-methyloxirane-2-carboxamide, which has following Formula:

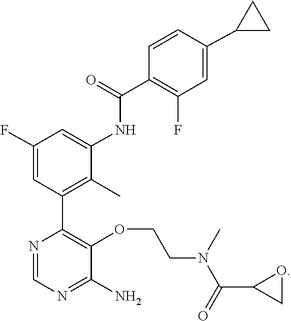 [SMILES: Cc1c(NC(=O)c2ccc(C3CC3)cc2F)cc(F)cc1-c1ncnc(N)c1OCCN(C)C(=O)C1CO1]

In another aspect, the invention provides a composition comprising or consisting of i) remibrutinib or a pharmaceutically acceptable salt thereof, wherein the total amount of epoxide impurity, e.g. the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in the composition is no more than about 75 ppm, e.g. no more than about 50 ppm, e.g. no more than about 30 ppm; or e.g. no more than about 7.5 ppm, relative to the total amount of remibrutinib in free or salt form.

In another aspect, the invention pertains to a composition comprising or consisting of remibrutinib or a pharmaceutically acceptable salt thereof, wherein the total amount of epoxide impurity, e.g. the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in the composition is no more than the maximum amount of epoxide that is permitted in such a composition, by a regulatory authority, at the time when the composition is prepared and/or administered.

In another aspect, the invention relates to a composition comprising or consisting of remibrutinib or a pharmaceutically acceptable salt thereof which has been tested and found to have a total amount of epoxides, e.g. a total amount of N-(2-((4-cyclopropyl-2-fluorobenzamido)-5-fluoro-2-methylphenyl)-pyrimidin-5-yl)-oxy)ethyl)-N-methyloxirane-2-carboxamide of no more than 30 ppm, relative to the total amount of remibrutinib in free or salt form.

In another aspect, the invention relates to a composition comprising or consisting of remibrutinib or a pharmaceutically acceptable salt thereof which has been tested and found to have a total amount of epoxides, e.g. a total amount of N-(2-((4-cyclopropyl-2-fluorobenzamido)-5-fluoro-2-methylphenyl)-pyrimidin-5-yl)-oxy)ethyl)-N-methyloxirane-2-carboxamide of no more than 7.5 ppm, relative to the total amount of remibrutinib in free or salt form.

In another aspect, the invention pertains to composition comprising or consisting of remibrutinib or a pharmaceutically acceptable salt thereof which has been tested and found to have a total amount of epoxide impurity, e.g. a total amount of N-(2-((4-cyclopropyl-2-fluorobenzamido)-5-fluoro-2-methylphenyl)-pyrimidin-5-yl)-oxy)ethyl)-N-methyloxirane-2-carboxamide, that is no more than the maximum amount of epoxides that is permitted in such a composition, by a regulatory authority, at the time when the composition is prepared and/or administered.

In another aspect, the invention relates to a pharmaceutical product comprising the composition as described above, i.e. substantially free of epoxide impurities, and instructions to a patient as to how to administer the composition.

In another aspect, the invention relates to a pharmaceutical product containing: (a) a composition comprising remibrutinib or a pharmaceutically acceptable salt thereof as defined above in term of epoxide impurities content; and (b) a document which certifies, either directly or via a link to an electronic database, that the total amount of epoxide impurities, e.g. the total amount of N-(2-((4-cyclopropyl-2-fluorobenzamido)-5-fluoro-2-methylphenyl)-pyrimidin-5-yl)-oxy)ethyl)-N-methyloxirane-2-carboxamide, in the composition is no more than about 75 ppm, no more than about 30 ppm, or no more than about 7.5 ppm, relative to the total amount of remibrutinib in free or salt form.

In another aspect, the invention relates to a pharmaceutical product containing: (a) a composition comprising remibrutinib or a pharmaceutically acceptable salt as defined above (substantially free of epoxide impurity); and (b) a document which certifies, either directly or via a link to an electronic database, that the total amount of epoxide impurities, e.g. the total amount of N-(2-((4-cyclopropyl-2-fluorobenzamido)-5-fluoro-2-methylphenyl)-pyrimidin-5-yl)-oxy)ethyl)-N-methyloxirane-2-carboxamide, in the composition is no more than the maximum amount of epoxide that is permitted in such a composition, by a regulatory authority, at the time when the composition is prepared and/or administered.

In a further aspect, the invention pertains to a pharmaceutical product as defined herein, wherein the product further comprises instructions to a patient as to how to administer the composition.

In another aspect, the invention pertains to a composition or pharmaceutical product as defined above, wherein the total amount of remibrutinib or a pharmaceutically acceptable salt thereof in the composition provides the same amount of remibrutinib as 10 mg, 25 mg or 100 mg of remibrutinib free base. In a further aspect, the invention relates to a composition or pharmaceutical product as defined above wherein the composition is an oral dosage form, optionally wherein the oral dosage form is a tablet. In yet another further aspect, the invention relates to a composition or pharmaceutical product as defined above wherein the composition consists of remibrutinib or a pharmaceutically acceptable salt thereof within an orally administrable capsule or tablet.

In another aspect, the invention relates to a composition or pharmaceutical product as defined above, wherein the composition further comprises one or more pharmaceutically acceptable excipients.

As used herein, the term “pharmaceutically acceptable excipients” includes any and all solvents, carriers, diluents, dispersion media, coatings, surfactants, antioxidants, preservatives (e.g., antibacterial agents, antifungal agents, antioxidants), isotonic agents, absorption delaying agents, salts, drug stabilizers, binders, additives, bulking agents, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). It should be understood that unless a conventional excipient is incompatible with the active ingredient, the use of any conventional excipient in any therapeutic or pharmaceutical compositions is contemplated by the present application.

The pharmaceutical composition can be formulated for particular routes of administration such as oral administration, parenteral administration, and rectal administration, etc. In addition, the pharmaceutical compositions described herein can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, carriers or buffering agents, as well as adjuvants, such as solvents, preservatives, stabilizers, wetting agents, emulsifiers and bulking agents, etc.

Typically, the pharmaceutical compositions are tablets or capsules comprising the active ingredient together with at least one excipient, such as:
a) diluents, e.g., lactose, dextrose, sucrose, mannitol, sorbitol, cellulose and/or glycine;
b) lubricants, e.g., silica, talcum, stearic acid, its magnesium or calcium salt, sodium stearyl fumarate and/or polyethyleneglycol; for tablets also
c) binders, e.g., magnesium aluminum silicate, starch paste, gelatin, tragacanth, methylcellulose, sodium carboxymethylcellulose, polyvinylpyrrolidone and/or, polyvinylpyrrolidone vinyl acetate copolymer; if desired;
d) carriers such as an aqueous vehicle containing a co-solvating material such as captisol, PEG, glycerin, cyclodextrin, or the like;
e) disintegrants, e.g., starches, agar, alginic acid or its sodium salt, croscarmellose sodium, crospovidone, sodium starch glycolate, or effervescent mixtures; and/or
f) absorbents, colorants, flavors and sweeteners.

Tablets may be either film coated or enteric coated according to methods known in the art. In one embodiment, the compound or composition is prepared for oral administration, such as a tablet or capsule, for example, and optionally packaged in a multi-dose format suitable for storing and/or dispensing unit doses of a pharmaceutical product. Examples of suitable packaging include, but are not limited to, hermetically sealed foils, unit dose containers (e.g., vials), blister packs, and strip packs.

Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed.

Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The present invention further comprises anhydrous pharmaceutical compositions and dosage forms comprising the remibrutinib prepared by or preparable by the methods described herein as active ingredients, since water may facilitate the degradation of certain compounds. Anhydrous pharmaceutical compositions and dosage forms can be prepared using anhydrous or low moisture containing ingredients and low moisture or low humidity conditions. An anhydrous pharmaceutical composition may be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further comprises pharmaceutical compositions and dosage forms that comprise one or more agents that reduce the rate by which the compound described herein as an active ingredient will decompose. Such agents, which are referred to herein as “stabilizers,” include, but are not limited to, antioxidants such as ascorbic acid, pH buffers, or salt buffers, etc.

In some embodiment, remibrutinib can be provided in a pharmaceutical composition, dosage form, and with pharmaceutical excipients as described in WO2022/162513 (Attorney docket number PAT059011-WO-PCT) which is hereby incorporated by reference in its entirety. In one embodiment the pharmaceutical composition is prepared according to the methods described herein (e.g. method of embodiments 16 to 40).

In another aspect, the invention relates to a composition or pharmaceutical product as defined above, wherein the composition further comprises one or more pharmaceutically acceptable excipients selected from a surfactant, a binder, a filler, a disintegrating agent, a lubricating agent and a gliding agent or mixture thereof.

The surfactant can be selected from sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof, preferably the surfactant is sodium lauryl sulfate.

The binder can be selected from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, vitamin E TPGS, or a mixture thereof, preferably the binder is polyvinylpyrrolidone-vinyl acetate copolymer (copovidone).

The filler can be selected from calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof preferably mannitol or cellulose or mixture thereof.

The disintegrating agent can be selected from croscarmellose sodium, crospovidone, sodium starch glycolate, corn starch, or alginic acid, or mixture thereof.

The lubricating agent can be selected from magnesium stearate, sodium stearyl fumarate, stearic acid or talc or mixture thereof.

In some embodiment, the pharmaceutical composition as described in WO2022/162513 further comprises one or more antioxidants. In one aspect of this embodiment, the one or more antioxidant is selected from Thymol, Propyl galate, Citric acid, Tartaric acid, ascorbic acid, Sodium ascorbate, gentisic acid, Mequinol, Butylated hydroxy toluene, butylated hydroxy anisole, sodium metabisulfite or mixture thereof, preferably Butylated hydroxy toluene, butylated hydroxy anisole, ascorbic acid, Sodium ascorbate, gentisic acid, sodium metabisulfite or mixture thereof, or more preferably ascorbic acid, sodium ascorbate and gentisic acid, or mixture thereof. Examples of such composition are described in any one of embodiments 57 to 83 herein. In another aspect of this embodiment, the composition is obtained according to the methods described herein (e.g. methods of any one of embodiments 16-40) The pharmaceutical composition described herein can be further formulated into a final dosage form (any one of embodiments 13, 84 and 85). Example of dosage form is capsule or tablet.

In yet another aspect, the invention relates to a composition or pharmaceutical product as defined above, wherein the composition is present in a sealed container together with desiccant.

In one aspect, the composition comprising or consisting of remibrutinib, is stored in the presence of a desiccant, and the composition is tested for the presence or amount of remibrutinib epoxide impurity.

The phrase “stored in the presence of a desiccant” as used herein refers to the inclusion of a desiccant within the packaging of the compositions and pharmaceutical products described. The phrase will be readily understood by a person of ordinary skill in the art who will be readily able to select suitable packaging. Examples of suitable packaging for the compositions and/or pharmaceutical products described herein include amber glass bottles, rigid high-density polyethylene (HDPE) bottles, Alu-bags, or blister packaging, such as rigid high-density polyethylene (HDPE) bottles, Alu-bags, or blister packaging. The desiccant may be placed in a separate container from the composition or pharmaceutical product, e.g. in a small HDPE canister or kraft paper bag, prior to inclusion in said packaging. The desiccant may form part of the packaging, e.g. the packaging may comprise a layer, a film, and/or a coating which consists of, comprises, or is derived from a desiccant and which is exposed to the void in which the composition or pharmaceutical product is contained. For example, the packaging may be a blister package that includes a blister tray that is a thermoformed plastic sheet with a plurality of blister cells or depressions formed therein. Typically, after the compositions or pharmaceutical products described herein are placed in the cells, the compositions or products are retained and protected in the respective cells by securing a backing sheet to the blister tray. One or more of the blister cells or depressions (e.g. each of the blister cells or depressions) may comprise a layer, a film and/or a coating which consists of, comprises, or is derived from a desiccant. Alternatively, or additionally, the backing sheet may comprise a layer, a film and/or a coating which consists, comprises, or is derived from a desiccant. Thus, in these embodiments, once the blister package is sealed, the composition is stored in said sealed blister package which also contains a desiccant as a component comprised within the blister package.

A “desiccant” as used herein refers to a hygroscopic material that absorbs moisture from the air and is used to create and maintain dry spaces i.e. to prevent condensation and maintain low humidity levels.

Suitable desiccants can readily be selected by a person of ordinary skill in the art. Examples of desiccants include activated alumina, aerogel, benzophenone, bentonite clay, calcium chloride, calcium oxide, calcium sulphate, cobalt(II) chloride, copper(II) sulphate, lithium chloride, lithium bromide, magnesium chloride hexahydrate, magnesium sulphate, magnesium perchlorate, molecular sieve, phosphorus pentoxide, potassium carbonate, potassium hydroxide, rice, silica gel, sodium, sodium chlorate, sodium chloride, sodium hydroxide, sodium sulphate, sucrose, sulfuric acid, triethylene glycol, and zeolite. For example, the desiccant may be selected from silica gel, molecular sieve, calcium oxide, and calcium sulphate, e.g. silica gel.

In one aspect, the invention provides a composition or pharmaceutical product as described herein, wherein said composition has a shelf life of at least 1, 2, 3, 4, 5, 6, 7, 8, 9, 10, 11, 12, 13, 14, 15, 16, 17, 18, 24, 30, 36, 42, 48, 54 or 60 months when stored at a temperature of no more than 40° C., preferably not more than 30° C., more preferably no more than 25° C., optionally at a temperature of 20 to 25° C. in a further aspect, the invention provides a composition or pharmaceutical product wherein said composition has a shelf life of at least 9 months.

In another aspect, the shelf life is the period of time in which the total amount of remibrutinib epoxide impurity (REI) in the composition remains no more than 30 ppm, relative to the total amount of remibrutibib in free or salt form.

In one aspect, the pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 10-50 mg of active ingredients. In an embodiment, the pharmaceutical composition or combination of the present invention can be in unit dosage of about 10 mg, about 25 mg or about 50 mg, or about 100 mg. The therapeutically effective dosage or amount of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The drug substance (i.e. remibrutinib drug substance) may be present in the pharmaceutical composition (e.g. film coated tablet (FCT)) in an amount of about 10 mg, about 15 mg, about 20 mg, about 25 mg, about 50 mg or about 100 mg.

In another aspect, the invention relates to a final dosage form comprising a pharmaceutical composition as described above and one or more pharmaceutically acceptable excipient(s). In one embodiment the final dosage form is a film coated tablet. In another embodiment, the final dosage form in a film coated tablet wherein LOU064 is present in an amount of about 5 mg, about 10 mg, about 15 mg, about 20 mg, about 25 mg or about 100 mg, e.g. about 25 mg or about 100 mg.

Manufacturing of Remibrutinib Drug Product (i.e. Composition Comprising Remibrutinib) Substantially Free of Epoxide Impurities

The invention further provides methods of preparing a pharmaceutical composition comprising (i) remibrutinib or a pharmaceutically acceptable salt thereof and (ii) one or more pharmaceutically acceptable excipients, wherein the pharmaceutical composition is substantially free of epoxide impurities, particularly epoxide impurity N-(2-((4-cyclopropyl-2-fluorobenzamido)-5-fluoro-2-methylphenyl)-pyrimidin-5-yl)-oxy)ethyl)-N-methyloxirane-2-carboxamide.

While methods of preparing such pharmaceutical compositions are described herein it will be readily apparent to a person of ordinary skill in the art that two or more of these methods may also be used in combination.

Thus, the present invention also provides the combined methods of preparing a pharmaceutical composition comprising (i) remibrutinib or a pharmaceutically acceptable salt thereof and (ii) one or more pharmaceutically acceptable excipients, wherein the pharmaceutical composition is substantially free of epoxide impurities, particularly N-(2-((4-cyclopropyl-2-fluorobenzamido)-5-fluoro-2-methylphenyl)-pyrimidin-5-yl)-oxy)ethyl)-N-methyloxirane-2-carboxamide. The pharmaceutical compositions produced by these methods may be used in the pharmaceutical products described herein.

It has been discovered that the epoxide formation occurred during the drug manufacturing process, specifically during the wet media milling process. The purpose of the wet media milling process is to reduce particle size of remibrutinib drug substance using mechanical energy input into a suspension by relative movement of the grinding media in a grinding chamber. The process parameters were found to be an impact on the particle size and the epoxide content.

Accordingly, the present invention provides a method of preparing a composition i) comprising remibrutinib or a pharmaceutically acceptable salt of remibrutinib and ii) one or more pharmaceutically acceptable excipients, wherein the method comprises a step of particle size reduction of remibrutinib using a wet media milling process wherein the wet media milling process is performed with a specific energy between about 600 and about 1000 KJ/Kg, preferably between about 600 and about 800 KJ/Kg.

In one aspect of this embodiment, the wet media milling process is performed in the presence of one or more antioxidants. The one or more antioxidant(s) is selected from Thymol, Propyl galate, Citric acid, Tartaric acid, ascorbic acid, Sodium ascorbate, gentisic acid, Mequinol, Butylated hydroxy toluene, butylated hydroxy anisole, sodium metabisulfite or combination thereof, preferably Butylated hydroxy toluene, butylated hydroxy anisole, ascorbic acid, sodium ascorbate, gentisic acid, sodium metabisulfite or combination thereof, or more preferably ascorbic acid, sodium ascorbate, gentisic acid or combination thereof. In another aspect of this embodiment, the antioxidant or mixture thereof are present in the wet media milling suspension in a total antioxidant amount of about 0.01% to about 0.5% weight by weight of the wet media milled suspension, preferably 0.01% to 0.2% w/w. In one aspect of this embodiment, the wet media milling process is performed in the presence of gentisic acid; e.g. gensitic acid is present in the wet media milling suspension in an amount of about 0.01% to 0.2% w/w. In another aspect of this embodiment, the wet media milling process is performed in the presence of sodium ascorbate, ascorbic acid or mixture thereof; e.g. sodium ascorbate, ascorbic acid or mixture thereof is present in the wet media milling suspension in an amount of about 0.01% to 0.5% w/w In one aspect, the present invention provides a method of preparing a composition comprising i) remibrutinib or a pharmaceutically acceptable salt of remibrutinib and ii) one or more pharmaceutically acceptable excipients wherein the wet media milling process as described above is performed at an average stress energy process parameter of no more than 1.3 pNm.

In another aspect, the invention provides a method as described above, wherein wet media milling process is performed in a grinding chamber with suspension outlet temperature of no more than 20° C.

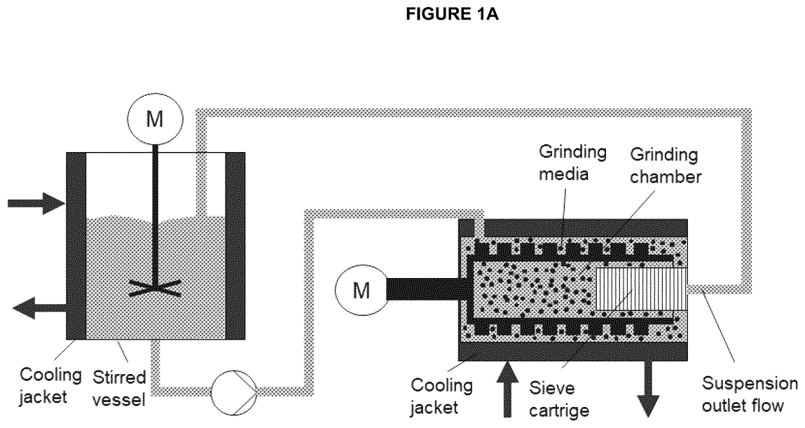

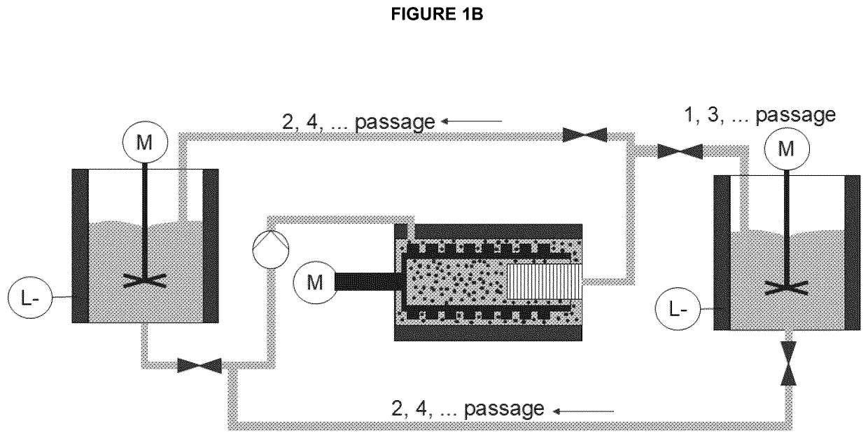

In another aspect, the invention provides a method as described above wherein the wet media milling process is performed in recirculation mode (as described in FIG. 1) with a turnover number of between about 28 to about 106.

In another aspect, the invention provides a method as described above wherein the wet media milling process is performed in a pendulum operation mode wherein the entire wet media suspension is pumped via the mill from a first vessel into a second vessel, and then the milled suspension is pumped via the mill back into the first vessel. The pumping backwards and forwards is continued until the desired target specific energy is met. This pendulum mode of operation allows for better control on the residence time distribution of the wet media milled suspension in the mill.

In another aspect, the invention provides a method of preparing a composition comprising i) remibrutinib or a pharmaceutically acceptable salt of remibrutinib and ii) one or more pharmaceutically acceptable excipients, as described above, wherein the wet media milling process generates a wet media milled suspension with a particle size of less or equal to 200 nm and wherein the composition is substantially free of epoxide impurities, e.g. substantially free of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide. The particle size is measured, for example, by laser diffraction methodology (e.g. particle size distribution (PSD)) using methods and instruments known to the skilled person in the art. For example, the particle size is measured analytical centrifugation (AC) or by photon correlation spectrometry (PCS).

In one aspect, the particle size is the mean particle sizes which are intensity weighted. The mean particle size can be determined by means of dynamic light scattering. For example, the mean particle size is determined by Photon Correlation Spectroscopy (PCS). In particular, for determining the mean particle size the device “Zetasizer Nano ZS”, Version 7.13 from Malvern Panalytical Ltd., UK can be used. In one aspect, the particle size is as determined in in WO2022/162513.

In another aspect, the particle size distribution is expressed in term of a polydispersity index (PI) which is between 0.01 and 0.5, more preferably between 0.09 and 0.2, in particular 0.09-0.15. A preferred particle size distribution is shown in Tables 5.2 to 5.4 and in Tables 10.3 to 10.5.

In another aspect, the invention provides a method of preparing a composition comprising i) remibrutinib or a pharmaceutically acceptable salt of remibrutinib and ii) one or more pharmaceutically acceptable excipients, as described above, wherein the wet media milling process generates a wet media milled suspension with a particle size of remibrutinib of less or equal to 200 nm and wherein the total of epoxide impurity, e.g. the total of N-(2-((4-amino-6-(3-(4-cyclopropyl-2-fluorobenzamido)-5-fluoro-2-methylphenyl)pyrimidin-5-yl)oxy)ethyl)-N-methyloxirane-2-carboxamide in the composition is no more than about 75 ppm, e.g. no more than about 50 ppm, e.g. no more than about 30 ppm; or e.g. no more than about 7.5 ppm, relative to the total amount of remibrutinib in free or salt form, preferably no more than 30 ppm, no more than 7.5 ppm.

The method of preparing a composition comprising i) remibrutinib or a pharmaceutically acceptable salt thereof, and ii) one or more pharmaceutically acceptable excipients, may further comprise one or more additional steps following the wet media milling step as described in WO2022/162513 (Attorney docket number PAT059011-WO-PCT) which is hereby incorporated by reference in its entirety.

In one aspect of the method of preparing a composition, the one or more pharmaceutically acceptable excipients may be selected from the group consisting of lactose, microcrystalline cellulose, mannitol, sucrose, starch, granulated hydrophilic fumed silica, polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, sodium lauryl sulfate, potassium lauryl sulfate, ammonium lauryl sulfate, sodium lauryl ether sulfate, polysorbates, perfluorobutanesulfonate, dioctyl sulfosuccinate, magnesium carbonate, kaolin, cellulose (e.g. microcrystalline cellulose, powdered cellulose), calcium phosphate, or sodium phosphate, croscarmellose sodium, crospovidone, sodium starch glycolate, corn starch, or alginic acid, magnesium stearate, sodium stearyl fumarate, stearic acid and talc.

In a further aspect of the method of preparing a composition, the one or more pharmaceutically acceptable excipients may be selected from the group consisting of magnesium stearate, sodium stearyl fumarate, microcrystalline cellulose, crospovidone, croscamellose sodium, lactose, mannitol, sodium lauryl sulfate, and polyvinylpyrrolidone-vinyl acetate copolymer (copovidone).

For example, in one aspect, the invention provides a method of preparing a composition comprising i) remibrutinib or a pharmaceutically acceptable salt of remibrutinib and ii) one or more pharmaceutically acceptable excipients as described above, said method further comprising an additional step of preparing suspension for a spray granulation step. In one aspect of this embodiment, the suspension for spray granulation comprises one or more antioxidant selected from Thymol, Propyl galate, Citric acid, Tartaric acid, ascorbic acid, sodium ascorbate, gentisic acid, Mequinol, Butylated hydroxy toluene, butylated hydroxy anisole, sodium metabisulfite or combination thereof, preferably Butylated hydroxy toluene, butylated hydroxy anisole, ascorbic acid, sodium ascorbate, gentisic acid, sodium metabisulfite or combination thereof, or more preferably ascorbic acid, sodium ascorbate, gentisic acid or combination thereof. In yet another aspect of this embodiment, the antioxidant or mixture thereof are present in the suspension for spray granulation in a total antioxidant amount of about 0.01% to about 0.5% weight by weight of the suspension for spray granulation, preferably 0.01% to 0.2% w/w. In one aspect of this embodiment, suspension for spray granulation comprises gentisic acid; e.g. gensitic acid is present in the spray granulation suspension in an amount of about 0.01% to 0.2% w/w. In another aspect of this embodiment, suspension for spray granulation comprises sodium ascorbate, ascorbic acid or mixture thereof; e.g. sodium ascorbate, ascorbic acid or mixture thereof is present in the spray granulation suspension in an amount of about 0.01% to 0.5% w/w In a further aspect of the method of preparing a composition, the media milled suspension and the suspension for spray granulation comprises one or more pharmaceutically acceptable excipients selected from a surfactant and a binder.

In another aspect of the method of preparing a composition, the media milled suspension and the suspension for spray granulation comprises a surfactant selected from sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof, preferably the surfactant is sodium lauryl sulfate.

In another aspect of the method of preparing a composition, the media milled suspension and the suspension of spray granulation comprises a binder selected from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, vitamin E TPGS, or a mixture thereof, preferably the binder is polyvinylpyrrolidone-vinyl acetate copolymer (copovidone).

In another aspect of the method of preparing a composition, one or more antioxidants as described above is/are added at the stage of preparing the wet media milling suspension and at the step of preparing the suspension for spray granulation.

In another aspect, the wet media milled suspension and spray granulation suspension are stored at a temperature of less than 25° C., e.g. less than 15° C., less than 10° C., e.g. between 5° C. and 8° C., optionally suspensions are tested for the presence or amount of remibrutinib epoxide impurity, prior to being used in the next step.

In another aspect, the invention provides a method of preparing a composition comprising i) remibrutinib or a pharmaceutically acceptable salt of remibrutinib and ii) one or more pharmaceutically acceptable excipients as described above, said method further comprising an additional step of preparing suspension for a spray granulation step; and wherein the suspension for spray granulation is sprayed onto an inert substrate to provide a granule.

In another aspect of the method of preparing a composition, the spray granulation is performed with a spray rate at steady state of between 500 and 600 g/min.

In another aspect of the method of preparing a composition, the spray granulation step is performed with an atomization air pressure of between 2.5 and 3.5 bar.

In another aspect of the method of preparing a composition, the inert substrate is selected from the group consisting of lactose, microcrystalline cellulose, mannitol, sucrose, starch, granulated hydrophilic fumed silica, or mixtures thereof, preferably the material, which is selected from the group consisting of lactose, mannitol, or mixtures thereof and most preferably the material is mannitol.

In another aspect, the invention provides a method of preparing a composition comprising i) remibrutinib or a pharmaceutically acceptable salt of remibrutinib and ii) one or more pharmaceutically acceptable excipients as described above, said method further comprising an additional step of:
preparing suspension for a spray granulation step; and
spraying the suspension onto an inert substrate to provide a granule, and
preparing a final blend.

In another aspect of the method of preparing a composition, the final blend is prepared by mixing the granule with one or more pharmaceutically acceptable excipients. In one aspect, the one or more pharmaceutically acceptable excipient for the final blend is selected from a filler, a disintegrating agent, a lubricating agent, and a gliding agent.

In another aspect of the method of preparing a composition, filler is selected from calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof preferably mannitol or cellulose or mixture thereof

In another aspect of the method of preparing a composition, the disintegrating agent is selected from croscarmellose sodium, crospovidone, sodium starch glycolate, corn starch, or alginic acid, or mixture thereof.

In another aspect of the method of preparing a composition, the lubricating agent is selected from magnesium stearate, sodium stearyl fumarate, stearic acid or talc or mixture thereof.

In another aspect, the invention provides a method of preparing a composition comprising i) remibrutinib or a pharmaceutically acceptable salt of remibrutinib and ii) one or more pharmaceutically acceptable excipients as described above, said method further comprising an additional step of:
preparing suspension for a spray granulation step; and
spraying the suspension onto an inert substrate to provide a granule, and
preparing a final blend, and
compressing the final blend into orally administrable dosage form, optionally a tablet.

Thus, an aspect of the invention relates to a method of preparing a composition comprising mixing remibrutinib or a pharmaceutically acceptable salt thereof with one or more pharmaceutically acceptable excipients, as described above wherein the one or more pharmaceutically acceptable excipients, and optionally each of the one or more pharmaceutically acceptable excipients, has a content of peroxide of less than 30 ppm, less than 20 ppm, or less than about 10 ppm. The one or more pharmaceutically acceptable excipients, e.g. each of the one or more pharmaceutically acceptable excipients, may have a content of peroxide of less than about 30 ppm, less than about 20 ppm, less than about 10 ppm.

Accordingly, the invention provides a method of preparing a composition as described above comprising mixing remibrutinib or a pharmaceutically acceptable salt thereof with one or more pharmaceutically acceptable excipients, wherein each of the one or more pharmaceutically acceptable excipients has a content of peroxide of less than 30 ppm, less than 20 ppm, or less than about 10 ppm, relative to the amount of the respective excipient. For example, the invention provides a method of preparing a composition as described above, wherein the copovidone as a binder has a peroxide content of less than 30 ppm, less than 20 ppm or less than 10 ppm.

Another aspect of the invention relates to a method of preparing a composition comprising mixing remibrutinib or a pharmaceutically acceptable salt thereof with more than one pharmaceutically acceptable excipient, as described above wherein the combined excipients have a total content of peroxide of less than 30 ppm, less than 20 ppm, or less than about 10 ppm relative to the combined amount of the excipients.

As disclosed elsewhere herein, the amount of peroxide in the composition, e.g. the amount of peroxide in a pharmaceutically acceptable excipient, may be determined using detection methods well known detection methods by the person skilled in the art, e.g. by high performance liquid chromatography (HPLC).

In one aspect, the invention further provides a method of preparing a composition as described herein, further comprising the use of antioxidant (e.g. ascorbic acid, sodium ascorbate, gentisic acid and the like) in order to prevent oxidation and/or reduce the amount of peroxide in the composition. The use of antioxidants can further reduce the amount of remibrutinib peroxide impurities, e.g. reduce the amount of N-(2-((4-cyclopropyl-2-fluorobenzamido)-5-fluoro-2-methylphenyl)-pyrimidin-5-yl)-oxy)ethyl)-N-methyloxirane-2-carboxamide.

Testing of the Drug substance and pharmaceutical composition (drug product) The invention further provides methods of testing the remibrutinib or pharmaceutically acceptable salt thereof, as well as methods of testing the compositions comprising (i) remibrutinib or a pharmaceutically acceptable salt thereof and (ii) one or more pharmaceutically acceptable excipients, for the presence and/or amount of epoxide impurities, in particular N-(2-((4-cyclopropyl-2-fluorobenzamido)-5-fluoro-2-methylphenyl)-pyrimidin-5-yl)-oxy)ethyl)-N-methyloxirane-2-carboxamide. Thus, an aspect of the invention relates to a method of evaluating a composition comprising remibrutinib or a pharmaceutically acceptable salt thereof, the method comprising testing the composition for the presence and/or amount of epoxide impurities, in particular impurity N-(2-((4-cyclopropyl-2-fluorobenzamido)-5-fluoro-2-methylphenyl)-pyrimidin-5-yl)-oxy)ethyl)-N-methyloxirane-2-carboxamide.

The method may be used to validate a process for the production of a composition comprising remibrutinib or a pharmaceutically acceptable salt thereof. Thus, an aspect of the invention relates to a method of validating a process for the production of a composition comprising remibrutinib or a pharmaceutically acceptable salt thereof, the method comprising testing the composition produced by said process for the presence and/or amount of epoxide impurities, in particular the presence and/or amount of N-(2-((4-cyclopropyl-2-fluorobenzamido)-5-fluoro-2-methylphenyl)-pyrimidin-5-yl)-oxy)ethyl)-N-methyloxirane-2-carboxamide.

Another aspect of the invention relates to a method of obtaining regulatory approval for a pharmaceutical composition which comprises or consists of remibrutinib or a pharmaceutically acceptable salt thereof, wherein the method comprises (i) testing the composition for the presence and/or amount of epoxide impurities, in particular the presence and/or amount of N-(2-((4-cyclopropyl-2-fluorobenzamido)-5-fluoro-2-methylphenyl)-pyrimidin-5-yl)-oxy)ethyl)-N-methyloxirane-2-carboxamide, and (ii) submitting the results of said testing to a regulatory authority. Suitable regulatory authorities to which the results may be submitted are, e.g. the FDA, the EMA, the MHRA, Swissmedic, or the PMDA.

In some aspects of the methods of the invention, a batch of the composition is tested to determine the presence and/or amount of epoxide impurities in said batch, in particular the presence and/or total amount of N-(2-((4-cyclopropyl-2-fluorobenzamido)-5-fluoro-2-methylphenyl)-pyrimidin-5-yl)-oxy)ethyl)-N-methyloxirane-2-carboxamide. In particular, a sample of the batch is tested. The batch testing may be used to determine whether to prepare a pharmaceutical product from said batch. For example, a pharmaceutical product may be prepared from the batch only if the batch is determined to have a total amount of epoxide impurities, e.g. a total amount of N-(2-((4-cyclopropyl-2-fluorobenzamido)-5-fluoro-2-methylphenyl)-pyrimidin-5-yl)-oxy)ethyl)-N-methyloxirane-2-carboxamide of no more than about 75 ppm, no more than about 50 ppm, no more than about 30 ppm, nor more than about 7.5 ppm, relative to remibrutinib in free or salt form.

Thus, in one aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of the composition,
b. determining the total amount of epoxide impurities, e.g. the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in said batch, in particular by testing a sample of the batch; and
c. preparing the pharmaceutical product from the batch only if the batch is determined to have a total amount of epoxide impurities, e.g. a total amount of N-(2-((4-cyclopropyl-2-fluorobenzamido)-5-fluoro-2-methylphenyl)-pyrimidin-5-yl)-oxy)ethyl)-N-methyloxirane-2-carboxamide of no more than about 75 ppm, no more than about 30 ppm, or no more than about 7.5 ppm, relative to remibrutinib in free or salt form.

In some aspects, a pharmaceutical product may be prepared from the batch only if the batch is determined to have a total amount of epoxide (e.g. total amount of N-(2-((4-cyclopropyl-2-fluorobenzamido)-5-fluoro-2-methylphenyl)-pyrimidin-5-yl)-oxy)ethyl)-N-methyloxirane-2-carboxamide) which would result in an amount of said epoxide impurities that corresponds to an amount of no more than 1.5 ug/day of total epoxide impurities (e.g. no more than 1.5 ug/day of N-(2-((4-cyclopropyl-2-fluorobenzamido)-5-fluoro-2-methylphenyl)-pyrimidin-5-yl)-oxy)ethyl)-N-methyloxirane-2-carboxamide) being administered to the patient when the composition is administered according to an approved dosage regimen of the remibrutinib or pharmaceutically acceptable salt thereof. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of the composition;
b. determining the total amount of epoxide impurities (e.g. N-(2-((4-cyclopropyl-2-fluorobenzamido)-5-fluoro-2-methylphenyl)-pyrimidin-5-yl)-oxy)ethyl)-N-methyloxirane-2-carboxamide) in said batch, in particular by testing a sample of the batch; and
c. preparing the pharmaceutical product from the batch only if the batch is determined to have a total amount of epoxide impurities (e.g. a total amount of N-(2-((4-cyclopropyl-2-fluorobenzamido)-5-fluoro-2-methylphenyl)-pyrimidin-5-yl)-oxy)ethyl)-N-methyloxirane-2-carboxamide) which would result in an amount of said epoxide impurities (e.g. an amount of N-(2-((4-cyclopropyl-2-fluorobenzamido)-5-fluoro-2-methylphenyl)-pyrimidin-5-yl)-oxy)ethyl)-N-methyloxirane-2-carboxamide) that corresponds to an amount of no more than 1.5 ug/day of epoxide impurities (such as no more than 1.5 ug/day of N-(2-((4-cyclopropyl-2-fluorobenzamido)-5-fluoro-2-methylphenyl)-pyrimidin-5-yl)-oxy)ethyl)-N-methyloxirane-2-carboxamide) being administered to the patient when the composition is administered according to an approved dosage regimen of the remibrutinib or pharmaceutically acceptable salt thereof.

In some aspects, a batch of the composition is tested to determine the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in said batch. In particular, a sample of the batch is tested. This batch testing may be used to determine whether to prepare a pharmaceutical product from said batch. For example, a pharmaceutical product may be prepared from the batch only if the batch is determined to have a total amount of N-(2-((4-cyclopropyl-2-fluorobenzamido)-5-fluoro-2-methylphenyl)-pyrimidin-5-yl)-oxy)ethyl)-N-methyloxirane-2-carboxamide is no more than about 75 ppm, no more than about 30 ppm, or no more than about 7.5 ppm, relative to remibrutinib in free or salt form. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of the composition;
b. determining the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in said batch, in particular by testing a sample of the batch; and
c. preparing the pharmaceutical product from the batch only if the batch is determined to have a total amount of N-(2-((4-cyclopropyl-2-fluorobenzamido)-5-fluoro-2-methylphenyl)-pyrimidin-5-yl)-oxy)ethyl)-N-methyloxirane-2-carboxamide is of no more than about 75 ppm, no more than about 30 ppm, or no more than about 7.5 ppm, relative to remibrutinib in free or salt form.

In some aspects, a batch of the composition is tested to determine the amount of total epoxide impurities, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, in said batch. In particular, a sample of the batch is tested. This batch testing may be used to determine whether to prepare a pharmaceutical product from said batch. For example, a pharmaceutical product may be prepared from the batch only if the batch is determined to have an amount of total epoxide impurities, including N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, of no more than about 75 ppm, no more than about 30 ppm, or no more than about 7.5 ppm, relative to remibrutinib in free or salt form.

In some aspects, a pharmaceutical product may be prepared from the batch only if the batch is determined to have an amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, which would result in an amount of said total epoxides that corresponds to an amount of no more than 1.5 ug/day of epoxide being administered to the patient when the composition is administered according to an approved dosage regimen of remibrutinib or pharmaceutically acceptable salt thereof. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of the composition;
b. determining the amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, in said batch, in particular by testing a sample of the batch; and
c. preparing the pharmaceutical product from the batch only if the batch is determined to have an amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, which would result in an amount of said total epoxides that corresponds to an amount of no more than 0.15 ug/day of epoxides being administered to the patient when the composition is administered according to an approved dosage regimen of remibrutinib or pharmaceutically acceptable salt thereof.

In some aspects, a batch of the wet media milled suspension is tested to determine the total amount of epoxide impurities (e.g. the total amount of N-(2-((4-cyclopropyl-2-fluorobenzamido)-5-fluoro-2-methylphenyl)-pyrimidin-5-yl)-oxy)ethyl)-N-methyloxirane-2-carboxamide) in said batch. In particular, a sample of the batch is tested. This batch testing may be used to determine whether to prepare a final composition from said batch, and therefore whether to prepare the pharmaceutical product from said batch. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of the wet media milled suspension;
b. determining the total amount of epoxide impurities, e.g. the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in said batch, in particular by testing a sample of the batch; and
c. preparing the pharmaceutical product from the batch only if the batch is determined to have a total amount of epoxide impurities, e.g. a total amount of N-(2-((4-cyclopropyl-2-fluorobenzamido)-5-fluoro-2-methylphenyl)-pyrimidin-5-yl)-oxy)ethyl)-N-methyloxirane-2-carboxamide of no more than about 75 ppm, no more than about 50 ppm, no more than about 30 ppm, no more than 15 ppm, no more than about 7.5 ppm or no more than about 2 ppm, relative to remibrutinib in free or salt form.

In some aspect, the wet media milled suspension is kept as a temperature of less than 20° C., during storage until it is used for preparing the composition as described herein.

In some aspect, stability testing may be performed using a sample of a batch of the composition.

Following this stability testing, the sample of the batch may be tested for the total amount of epoxides (such as the total amount of N-(2-((4-cyclopropyl-2-fluorobenzamido)-5-fluoro-2-methylphenyl)-pyrimidin-5-yl)-oxy)ethyl)-N-methyloxirane-2-carboxamide). This testing of the sample of the batch may be used to determine whether the batch is suitable for distribution and/or administration to a patient. For example, the batch may be determined to be suitable for distribution only if the sample of the batch after stability testing is determined to have a total amount of epoxides (such as a total amount of N-(2-((4-cyclopropyl-2-fluorobenzamido)-5-fluoro-2-methylphenyl)-pyrimidin-5-yl)-oxy)ethyl)-N-methyloxirane-2-carboxamide) of no more than about 75 ppm, no more than about 30 ppm, or no more than about 7.5 ppm, relative to remibrutinib in free or salt form

Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of epoxides (such as total amount of N-(2-((4-cyclopropyl-2-fluorobenzamido)-5-fluoro-2-methylphenyl)-pyrimidin-5-yl)-oxy)ethyl)-N-methyloxirane-2-carboxamide) in the sample of the batch after stability testing; and
d. validating the batch for distribution only if the sample of the batch after stability testing is determined to have a total amount of epoxides (such as total amount of N-(2-((4-cyclopropyl-2-fluorobenzamido)-5-fluoro-2-methylphenyl)-pyrimidin-5-yl)-oxy)ethyl)-N-methyloxirane-2-carboxamide) of no more than about 75 ppm, no more than about 30 ppm, or no more than about 7.5 ppm, relative to remibrutinib in free or salt form.

In some aspects, stability testing may be performed using a sample of a batch of the composition and the sample of the batch may be tested for the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in said sample of the batch after stability testing. This testing of the sample of the batch may be used to determine whether the batch is suitable for distribution and/or administration to a patient. For example, the batch may be determined to be suitable for distribution only if the sample of the batch after stability testing is determined to have a total amount of N-(2-((4-cyclopropyl-2-fluorobenzamido)-5-fluoro-2-methylphenyl)-pyrimidin-5-yl)-oxy)ethyl)-N-methyloxirane-2-carboxamide of no more than about 75 ppm, no more than about 30 ppm, or no more than about 7.5 ppm, relative to remibrutinib in free or salt form. Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of N-(2-((4-cyclopropyl-2-fluorobenzamido)-5-fluoro-2-methylphenyl)-pyrimidin-5-yl)-oxy)ethyl)-N-methyloxirane-2-carboxamide in said sample of said batch after stability testing; and
d. validating the batch for distribution only if the sample of the batch after stability testing is determined to have a total amount of N-(2-((4-cyclopropyl-2-fluorobenzamido)-5-fluoro-2-methylphenyl)-pyrimidin-5-yl)-oxy)ethyl)-N-methyloxirane-2-carboxamide of no more than about 75 ppm, no more than about 30 ppm, or no more than about 7.5 ppm, relative to remibrutinib in free or salt form.

In some aspects, stability testing may be performed using a sample of a batch of the composition and the sample of the batch may be tested for the amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, in said batch after stability testing. This testing of the sample of the batch may be used to determine whether the batch is suitable for distribution and/or administration to a patient. For example, the batch may be determined to be suitable for distribution only if the sample of the batch after stability testing is determined to have an amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, of no more than about 75 ppm, no more than about 50 ppm, no more than about 30 ppm, or no more than about 7.5 ppm, relative to remibrutinib in free or salt form. Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, in said batch after stability testing; and
d. validating the batch for distribution only if the sample of the batch after stability testing is determined to have an amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, of no more than about 75 ppm, no more than about 30 ppm, or no more than about 7.5 ppm, relative to remibrutinib in free or salt form.

In some aspects, the batch may be determined to be suitable for distribution only if the sample of the batch after stability testing is determined to have an amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, which would result in an amount of said total epoxides which corresponds to an amount of no more than 0.15 ug/day/day of epoxides being administered to the patient when the composition is administered according to an approved dosage regimen of remibrutinib or pharmaceutically acceptable salt thereof. Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, in the sample of the batch after stability testing; and
d. validating the batch for distribution only if the sample of the batch after stability testing is determined to have an amount of total epoxides, including both N-(2-((4-cyclopropyl-2-fluorobenzamido)-5-fluoro-2-methylphenyl)-pyrimidin-5-yl)-oxy)ethyl)-N-methyloxirane-2-carboxamide and other epoxides, which would result in an amount of said total epoxides which corresponds to an amount of no more than 0.15 ug/day of epoxides being administered to the patient when the composition is administered according to an approved dosage regimen of remibrutinib or pharmaceutically acceptable salt thereof.

In any of these methods, the step of testing for the presence and/or amount of epoxides may be performed using high performance liquid chromatography (HPLC)- and/or gas chromatography (GC)-mass spectroscopy. Likewise, the step of determining the total amount of N-(2-((4-cyclopropyl-2-fluorobenzamido)-5-fluoro-2-methylphenyl)-pyrimidin-5-yl)-oxy)ethyl)-N-methyloxirane-2-carboxamide may be performed using high performance liquid chromatography (HPLC) and/or gas chromatography (GC)-mass spectroscopy. For example, the HPLC-MS method performed may be the method provided in Example 2.

The invention also provides for the use of N-(2-((4-cyclopropyl-2-fluorobenzamido)-5-fluoro-2-methylphenyl)-pyrimidin-5-yl)-oxy)ethyl)-N-methyloxirane-2-carboxamide as a reference standard to detect an impurity in a composition comprising remibrutinib or a pharmaceutically acceptable salt thereof. The composition may comprise remibrutinib or a pharmaceutically acceptable salt thereof and one or more pharmaceutically acceptable excipients. The impurity may be N-(2-((4-cyclopropyl-2-fluorobenzamido)-5-fluoro-2-methylphenyl)-pyrimidin-5-yl)-oxy)ethyl)-N-methyloxirane-2-carboxamide. The detection of the impurity may be performed using high performance liquid chromatography (HPLC)- and/or gas chromatography (GC)-mass spectroscopy.

### Medical Uses

An aspect of the invention provides compositions or pharmaceutical products of the invention for use in methods of therapy. The compositions or pharmaceutical products of the invention may be for use in the treatment of disorders mediated by BTK or ameliorated by the inhibition of BTK. The methods may comprise administering a therapeutically effective amount of remibrutinib or a pharmaceutically acceptable salt thereof, in a composition or pharmaceutical product of the invention to a patient in need thereof.

It will be understood by a person skilled in the art that the invention also encompasses a method of treatment, including a method of treating disorders mediated by BTK or ameliorated by the inhibition of BTK, by administering to a patient in need thereof a therapeutically effective amount of remibrutinib or a pharmaceutically acceptable salt thereof in a composition or pharmaceutical product of the invention.

The invention also encompasses a pharmaceutical composition comprising remibrutinib or a pharmaceutically acceptable salt thereof for use in therapy, including for use in the treatment of disorders mediated by BTK or ameliorated by inhibition of BTK. The invention also encompasses the use of a composition or pharmaceutical product of the invention for the manufacture of a medicament for the treatment of a disease, including for the treatment of disorders mediated by BTK or ameliorated by inhibition of BTK as described herein. The invention also encompasses the use of a composition or pharmaceutical product of the invention for therapy, including for treating disorders mediated by BTK or ameliorated by inhibition of BTK as described herein.

The present invention also comprises a method for the treatment of disorders mediated by BTK or ameliorated by the inhibition of BTK, comprising administering to a patient in need of such treatment a therapeutically effective amount of remibrutinib, in a composition prepared by or preparable by a method described herein (composition substantially free of N-(2-((4-cyclopropyl-2-fluorobenzamido)-5-fluoro-2-methylphenyl)-pyrimidin-5-yl)-oxy)ethyl)-N-methyloxirane-2-carboxamide as described herein).

Also included in the invention are methods of treatment disorders mediated by BTK or ameliorated by the inhibition of BTK, comprising administering to a patient in need of such treatment, a pharmaceutical composition substantially free of N-(2-((4-cyclopropyl-2-fluorobenzamido)-5-fluoro-2-methylphenyl)-pyrimidin-5-yl)-oxy)ethyl)-N-methyloxirane-2-carboxamide as described herein.

Composition, pharmaceutical products prepared by or preparable by a method described herein (e.g. substantially free of epoxide impurities, (e.g. substantially free of N-(2-((4-cyclopropyl-2-fluorobenzamido)-5-fluoro-2-methylphenyl)-pyrimidin-5-yl)-oxy)ethyl)-N-methyloxirane-2-carboxamide as described herein) are useful in the treatment of the following diseases or disorders mediated by BTK or ameliorated by inhibition of BTK: Autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis especially relapsing multiple sclerosis, myasthenia gravis, Sjogren's syndrome, hidradenitis suppurativa, IgE driven allergy, e.g. drug, venom, food allergy; autoimmune hemolytic anemia, anti-neutrophil cytoplasmic antibodies (ANCA)-associated vasculitis, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerulonephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of hematopoietic origin including, but not limited to, multiple myeloma; a leukemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenström disease.

Composition, pharmaceutical products prepared by or preparable by a method described herein (substantially free of epoxide impurities, e.g. substantially free of N-(2-((4-cyclopropyl-2-fluorobenzamido)-5-fluoro-2-methylphenyl)-pyrimidin-5-yl)-oxy)ethyl)-N-methyloxirane-2-carboxamide as described herein) is especially useful in the treatment of chronic urticaria, e.g. chronic spontaneous urticaria or chronic inducible urticaria; Sjogren's syndrome, multiple sclerosis, hidradenitis suppurativa and food allergy.

### EXAMPLES

During product development of the pharmaceutical composition comprising LOU064, as disclosed in WO/2022/162513, the presence of a product degradation (epoxide) impurity was detected in LOU064 drug product. The epoxide impurity was found AMES positive and was considered a genotoxic impurity (GTI).

Investigations revealed that the epoxide impurity significantly increased during the wet media milling operation. This finding has triggered a reassessment of the wet media milling process parameters with the aim to identify a new set of operational parameter ranges that ensure the quality of the output material, while controlling the levels of the epoxide below the proposed specification based on ICH M7.

TABLE 1

| | | Maximum dose | Daily intake | Limit |
|---|---|---|---|---|
| | | daily (9 mg/day) | of epoxide | epoxide |
| | Dose (mg) | (BID) | (ug/day) | (ppm) |
| Clinic | 10 | 20 | 10 (>1-10 | 500 |
| setting | 25 | 50 | years) | 200 |
| | 100 | 200 | | 50 |
| Commercial | 10 | 20 | 1.5 (>10 | 75 |
| setting | 25 | 50 | years) | 30 |
| | 100 | 200 | | 7.5 |

While the pharmaceutical composition disclosed in WO/2022/162513 (attorney docket number PAT059011) met the clinical setting requirements (e.g. for dose 25 mg B.I.D.), new process of manufacture is needed to meet the commercial setting requirements as determined by health authorities, e.g. less than 75 ppm, less than 30 ppm, or less than 7.5 ppm. (table 1)

### Example 1

Enhanced Ames Test (EAT) for N-(2-((4-cyclopropyl-2-fluorobenzamido)-5-fluoro-2-methylphenyl)-pyrimidin-5-yl)-oxy)ethyl)-N-methyloxirane-2-carboxamide (Remibrutinib epoxide impurity referred to as REI hereinafter)

N-(2-((4-cyclopropyl-2-fluorobenzamido)-5-fluoro-2-methylphenyl)-pyrimidin-5-yl)-oxy)ethyl)-N-methyloxirane-2-carboxamide was evaluated in the EAT performed according to regulatory test guidance.

REI was tested in a Non-GLP Ames test using Salmonella strains TA98, TA100, TA97a and TA102 in the presence and absence of 10% rat-S9 metabolic activation (study 2412501). The test used plate-incorporation method. REI was formulated in dimethyl sulfoxide (DMSO) at concentrations of 5, 16, 50, 160, 500, 1600 and 5000 pg/plate.

No precipitation was observed in the study. Evidence of toxicity in the form of a marked reduction in revertant numbers was observed at 1600 pg/plate and above in strain TA1535 in the absence of S-9 and at 5000 μg/plate in strains TA98 and TA97a in the absence and presence of S-9.

The strain-specific positive controls induced increases in revertant numbers of 1.5-fold (in strain TA102), 2-fold (in strains TA98, TA100 and TA97a) or 3-fold (for strain TA1535) the concurrent vehicle controls confirming validity of the different strains in the presence and absence of S9. Following treatment with REI, increases in revertant numbers of at least 2-fold the concurrent vehicle controls were observed in strain TA97a in the absence of S-9. No other increases in revertant numbers of at least 2-fold (1.5-fold for strain TA102 or 3-fold for strain TA1535) the concurrent vehicle controls were observed following any other strain treatments.

In conclusion, REI showed evidence of a mutagenic potential in strain TA97a in the absence of S-9 but did not show evidence of a mutagenic potential in strains TA98, TA100, TA102 and TA1535 in the absence and presence of S-9, or in strain TA97a in the presence of S-9 when tested up to 5000 μg/plate as required by current regulatory test guidance.

It is therefore concluded that REI was mutagenic in the Ames test in the absence of metabolic activation.

It is therefore necessary to decrease the content of epoxide impurity in the drug product (DP)

### Example 2

Validation method for amount of N-(2-((4-cyclopropyl-2-fluorobenzamido)-5-fluoro-2-methylphenyl)-pyrimidin-5-yl)-oxy)ethyl)-N-methyloxirane-2-carboxamide in LOU064 drug substance and LOU064 film coated tablet
Specific impurity REI is quantified by reverse phase HPLC with mass spectrometry (MS) detection (electrospray in positive mode)

### Reagents:

| | |
|---|---|
| Formic acid (≥99%) | LCMS grade or equivalent |
| | (e.g. Fisher Chemical, Cat. no A117-50) |
| Acetonitrile (ACN) | LCMS grade or equivalent |
| | (e.g. Supelco, hypergrade for LC-MS LiChrosolv, |
| | code no. 1.00029) |
| Methanol | LCMS grade or equivalent |
| | (e.g. Fisher Chemical, Cat. no A456-4) |
| Water | HPLC grade or equivalent (e.g. Milli-Q water) |
| Impurity reference | REI |
| Resolution reference | LOU064 |

### Reagent Preparation

Water/Acetonitrile (80/20, v/v)

For example, transfer 800 mL of water and 200 mL of acetonitrile in a 1L mobile phase bottle and mix well.

### Equipment Material

| Apparatus | HPLC or UHPLC system with binary or quaternary pump |
|---|---|
| | with at least 600 bar back pressure, equipped with column |
| | oven, auto-sampler, and mass detector with electrospray |
| | ionization (ESI) source, e.g., Agilent 1260 Infinity UHPLC |
| | system with Agilent G6125B mass spectrometer or equivalent |
| Column | Agilent Zorbax Bonus-RP RRHD, particle size: 1.8 μm, |
| | length 100 mm, internal diameter 2.1 mm (Part-No |
| | 858768-901) |

### Operating Parameters

| | |
|---|---|
| Mobile | Water/Acetonitrile/Formic Acid (95/5/0.1 v/v/v) |
| phase A | For example, transfer 950 mL of water and 50 mL of acetonitrile |
| | in a 1 L mobile phase bottle. Add 1.0 mL of formic acid, mix |
| | well. |
| Mobile | Water/Acetonitrile/Methanol/Formic Acid (5/50/45/0.1 v/v/v/v) |
| phase B | For example, transfer 50 mL of water, 500 mL acetonitrile and |
| | 450 mL of methanol in a 1 L mobile phase bottle. Add 1.0 mL |
| | of formic acid, mix well. |

### Gradient Refer to Table 2

TABLE 2
Gradient

| Time (min) | Mobile phase A (%) | Mobile phase B (%) |
|---|---|---|
| 0.0 | 95 | 5 |
| 1.0 | 95 | 5 |
| 16.0 | 40 | 60 |
| 18.1 | 5 | 95 |
| 20.0 | 5 | 95 |
| 20.1 | 95 | 5 |
| 24.0 | 95 | 5 |

| | |
|---|---|
| Flow rate | 0.4 mL/min |
| Detection mode | Mass spectrometry (MS) with electrospray |
| | ionization (ESI), refer to section 1.2 |
| Column temperature | 45° C. |

| | | |
|---|---|---|
| Time (min) | Mobile phase A (%) | Mobile phase B (%) |

| | |
|---|---|
| Auto sampler temperature | 5° C. ± 3° C. |
| Injection volume | 10 μL |
| Run time | 24 min |
| Needle wash | Acetonitrile |

MS Conditions The instrument settings described in this test method apply to the Agilent G6125B mass spectrometer. Other instruments providing adequate or improved method performance may be used instead and MS conditions can be adjusted to optimize the method sensitivity.

| | |
|---|---|
| Polarity | ESI Positive |
| Mass range | Single ion monitoring (SIM) 524.2 m/z |
| Scan time | The MS acquisition can be started with a delay of 9.0 minutes and |
| | switched off again after 13.5 min. During non-acquisition time eluent |
| | flow will be conducted to the waste. |
| | Note that the MS acquisition range may be adapted to fit the |
| | instrument configuration used. In any case, it should be ensured that |
| | the peak associated with REI is appropriately quantified whilst |
| | avoiding detector saturation due to the LOU064 Drug substance |
| | elution. Care should also be taken that the signal-to-noise can be |
| | measured using 5 times the width at peak half height situated equally |
| | around the REI peak retention time (as per system suitability test |
| | (SST)). |
| Fragmentor | 100 V |
| Capillary voltage (Positive) | 3500 V |
| Drying Gas | 10.0 L/min |
| Gas temperature | 300° C. |
| Nebulizer pressure | 35 psig |

### Test Procedure/Preparation of Solutions

### Reference Solution Preparation

| | | |
|---|---|---|
| Number of solutions to be | Reference stock solution (RSS) | 2 preparations each from |
| prepared | | independent weighing |
| | Reference solution | 2 preparations each from |
| | (RS_1/RS_2) | independent weighing of |
| | | reference stock solutions |

| | |
|---|---|
| Reference stock solution in | Prepare reference stock solution of 0.2 μg/mL of REI impurity |
| duplicate (RSS_1 and RSS_2) | reference by dissolving it in acetonitrile and further diluting with |
| (concentration: 500 ppm of REI | sample solvent. |
| free form with respect to 0.4 | For example, weigh approximately 10 mg of REI impurity |
| mg/mL of LOU064 free form) | reference, accurately to at least 0.001 mg, into a 100 mL |
| | volumetric flask and dilute up to the mark with acetonitrile with |
| | the help of sonication if required. Mix well. Sonicate, if necessary. |
| | Afterwards pipette 1.0 mL of this solution into a 20 mL volumetric |
| | flask. Dilute to volume with sample solvent and mix well. |
| | Finally, pipette 4.0 mL of this solution into a 100 mL volumetric |
| | flask. Dilute to volume with sample solvent and mix well. |
| Reference solution in duplicate | Pipette 2.5 mL of the Reference stock solution (RSS_1) into a |
| for LOU064 50 and 100 mg | 25-mL volumetric flask. Dilute to volume with sample solvent and |
| FCTs | mix well. |
| (RS50(or100)_1 and | (Concentration: 20 ng/mL) |
| RS50(or100)_2) | |
| (REI: 50 ppm) | |
| Reference solution in duplicate | Pipette 1.5 mL of the Reference stock solution (RSS_1) into a |
| for LOU064 25 mg FCTs | 25-mL volumetric flask. Dilute to volume with sample solvent and |
| (RS25_1 and RS25_2) | mix well. |
| (REI: 30 ppm) | (Concentration: 12 ng/mL) |
| Reporting limit solution (RL) | Pipette 1.0 mL of the reference stock solution (RSS_1) into a 250 |
| (concentration REI: 2 ppm wrt | mL volumetric flask. Dilute to volume with sample solvent and |
| 0.4 mg/mL) | mix well. |
| | (Concentration: 0.8 ng/mL) |
| Resolution solution | Transfer 1 film coated tablet of LOU064 25 mg_SST1 into a 25 |
| | mL volumetric flask. Add 7.5 mL water and agitate in a |
| | mechanical shaker until complete disintegration of the tablets |
| | (e.g. 150 rpm, 15 minutes). Add about 12.5 mL of acetonitrile and |
| | equilibrate this solution until it reaches room temperature and |
| | dilute up to the mark with acetonitrile. Mix well. Add a magnetic |
| | stirrer bar to flask and stirred for 30 minutes at 600 rpm. |
| | Centrifuge a portion of the sample appropriately e.g. at 4000 rpm |
| | for about 10 minutes. Pipette 10.0 mL of the clear supernatant |
| | into a 25 mL volumetric flask and dilute up to the mark with |
| | sample solvent. |
| Stability of solutions | The reference and resolution solutions are stable for up to 14 |
| | days at ambient temperature in clear glass volumetric flask. |
| Note | For reference solutions alternative volumetric flask sizes and |
| | dilutions may be employed as long as the final concentrations |
| | are maintained. |

### Test Solution (TS) Preparation

| | |
|---|---|
| Number of solutions to be | 2 preparations |
| prepared | |
| Test solution for drug product | Transfer 10 film-coated tablets of LOU064 into a 250 mL volumetric |
| LOU064 25 MG Film Coated | flask. Add 75 mL water and agitate in a mechanical shaker until |
| Tablets | complete disintegration of the tablets (e.g. 150 rpm, 15 minutes). |
| | Add about 125 mL of acetonitrile and equilibrate this solution until it |
| | reaches room temperature and dilute up to the mark with |
| | acetonitrile. Mix well. Add a magnetic stirrer bar to flask and stirred |
| | for 30 minutes at 600 rpm. |
| | Centrifuge a portion of the sample appropriately e.g. at 4000 rpm |
| | for about 10 minutes. Pipette 10.0 mL of the clear supernatant into |
| | a 25 mL volumetric flask and dilute up to the mark with sample |
| | solvent. (Final concentration of LOU064 is ~0.4 mg/mL). |
| Test solution for drug product | Transfer 10 film-coated tablets of LOU064 into a 500 mL volumetric |
| LOU064 50 MG Film Coated | flask. Add 150 mL water and agitate in a mechanical shaker until |
| Tablets | complete disintegration of the tablets (e.g. 150 rpm, 15 minutes). |
| | Add about 250 mL of acetonitrile and equilibrate this solution until it |
| | reaches room temperature and dilute up to the mark with |
| | acetonitrile. Mix well. Add a magnetic stirrer bar to flask and stirred |
| | for 30 minutes at 600 rpm. |
| | Centrifuge a portion of the sample appropriately e.g. at 4000 rpm |
| | for about 10 minutes. Pipette 10.0 mL of the clear supernatant into |
| | a 25 mL volumetric flask and dilute up to the mark with sample |
| | solvent. (Final concentration of LOU064 is ~0.4 mg/mL). |
| Test solution for drug product | Transfer 10 film-coated tablets of LOU064 into a 1000 mL |
| LOU064 100 MG Film Coated | volumetric flask. Add 300 mL water and agitate in a mechanical |
| Tablets | shaker until complete disintegration of the tablets (e.g. 150 rpm, 15 |
| | minutes). Add about 500 mL of acetonitrile and equilibrate this |
| | solution until it reaches room temperature and dilute up to the mark |
| | with acetonitrile. Mix well. Add a magnetic stirrer bar to flask and |
| | stirred for 30 minutes at 600 rpm. |
| | Centrifuge a portion of the sample appropriately e.g. at 4000 rpm |
| | for about 10 minutes. Pipette 10.0 mL of the clear supernatant into |
| | a 25 mL volumetric flask and dilute up to the mark with sample |
| | solvent. (Final concentration of LOU064 is ~0.4 mg/mL). |
| Stability of solution | The test solution is stable for up to 14 days at ambient temperature |
| | in clear glass volumetric flask. |
| Note | For test solutions the extraction volume cannot be changed, but for |
| | dilutions of the test solutions alternative volumetric flask sizes and |
| | dilutions may be employed as long as the final concentrations are |
| | maintained. |

### Analysis

### System Suitability Test (SST)

| | |
|---|---|
| Note | The assessment of the SST attributes and the |
| | calculations are performed in accordance with |
| | Ph. Eur. 2.2.46, USP <621>, JP 2.00 and JP 2.01 |
| | on ′Chromatography′ as well as in accordance |
| | with the local SOP. |
| | The SST attribute ′Blank evaluation′ must be |
| | assessed at least at the start of the sequence. |
| | The SST attribute Signal-to-noise ratio must be |
| | assessed at least at the start and the end of the |
| | sequence. |
| Blank evaluation | No peak in the sample solvent and reference |
| | standard solvent chromatogram interfering |
| | with the peak of impurity REI with an area |
| | equal or greater than 1/3 of the peak area of REI |
| | in the reporting limit solution (RL). |
| Repeatability | srel ≤ 20% tested at the beginning of the sequence |
| | using the RS_1 at the beginning (n = 5) and |
| | across the entire sequence. |
| Standard accuracy | The standard agreement between the two |
| | preparations of the reference solution (RS_1 and |
| | RS_2) must be within the range of 90% to 110%. |

$\begin{matrix}Standard agreement = \\ \frac{Mean peak area of RS\_ 1}{Mean peak area of RS\_ 2} \times \frac{Reference weight of RS\_ 2}{Reference weight of RS\_ 1} \times 100\end{matrix}$

| | |
|---|---|
| Peak-to-valley | Peak-to-valley ratio ≥1.0, between the REI peak and the |
| | subsequent peak in the resolution solution. Two peaks must be |
| | clearly distinguished. |
| Signal-to-noise ratio | Signal-to-noise ≥10 (peak of REI in the reporting limit solutions, |
| | (RL)) |
| | The S/N ratio is determined based on a distance equal to at least 5 |
| | times the width at peak half height situated equally around the |
| | peak retention time. |
| Evaluation | |
| Evaluation | Determine the peak area for REI in the chromatograms of the test |
| | solution(s) and the peak area for REI in the chromatograms of |
| | reference solution. |
| Calibration mode | Total: The mean peak response of all injections of reference |
| | solution is used for calibration. |

### Calculation

| Calculation | Content of REI [ppm]: |
|---|---|
| | $C = \frac{( {PA}_{T} \times {m}_{R} \times D \times {C}_{R} )}{( {PA}_{R} \times {V}_{R} \times F \times 100 )} \times 1000000$ |
| PA_{T} | Peak area of REI in the test solution [counts · min] |
| m_{R} | Mass of test substance in reference solution [mg] |
| D | Dilution |
| | For drug product LOU064 25MG Film Coated Tablets |
| | (FCT): 0.00012 |
| | For drug product LOU064 50MG Film Coated Tablets: |
| | 0.0002 |
| | For drug product LOU064 100MG Film Coated |
| | Tablets: 0.0002 |
| C_{R} | Declared content of REI reference standard in percent |
| PA_{R} | Peak area of REI in the reference solution [counts · |
| | min] |
| V_{R} | Volume of reference stock solution [mL] |
| F | For drug product LOU064 25MG Film Coated Tablets: |
| | 0.4 |
| | For drug product LOU064 50MG Film Coated Tablets: |
| | 0.4 |
| | For drug product LOU064 100MG Film Coated |
| | ablets: 0.4 |

### Example 3: Manufacturing LOU064 Tablets

During the course of LOU064 drug development, it was uncovered that the genotoxic impurity (REI) content increases upon drug product manufacturing process. The drug manufacturing process is described below.

The objective of the study was therefore to evaluate the impact of selected material attributes and/or equipment and process parameters on LOU064 wet media milled suspension purity profile, in particular regarding particle size and degradation product REI.

### 1. Manufacturing Description

### Wet Media Milling

1. Dissolve copovidone in purified water using a vessel with stirrer
2. Add sodium lauryl sulfate to the solution from step 1 and dissolve under stirring
3. Add LOU064 (remibrutinib) drug substance to the solution from step 2 and suspend under stirring
4. Perform wet media milling on the suspension from step 3 to obtain wet media milled suspension, until a target specific energy

### Spray Granulation

5. Dissolve copovidone in purified water using a vessel with stirrer
6. Add sodium lauryl sulfate to the solution from step 5 and dissolve under stirring
7. Combine wet media milled suspension from step 4 and solution of copovidone and sodium lauryl sulfate from step 6 to complete the suspension for spray granulation; stir the mixture until a homogeneous suspension is obtained
8. Load the fluid bed granulator with mannitol and perform spray granulation by spraying the suspension from step 7 onto the mannitol. Dried granules are finally obtained

### Screening

9. De-agglomerate and sieve the dried raw granules from step 8, cellulose microcrystalline, mannitol, croscarmellose sodium and sodium stearyl fumarate through a screening mill (1.0 mm screen)

### Blending

10. Blend the sieved materials from step 9 in a diffusion mixer to obtain the final blend

### Tableting

11. Compress the final blend from step 10 using a rotary tablet press with a compression force adapted to achieve mean tablet hardness

### Coating

12. Prepare a homogeneous suspension of coating premixes in purified water in a suitable sized vessel.
13. Film-coat the tablet cores from Step 11 with the film coat suspension from step 12 in a perforated coating pan to achieve desired film-coat weight gain, followed by drying and cooling. Collect the film coated tablets in a suitable container

The formulation used for all experiments was the same as for the commercial process, e.g. for 25 mg film coated tablets
25% w/w LOU064 DS, 4% w/w Copovidone, 0.1% w/w Sodium Lauryl Sulfate for wet media milling.
Final tablet composition

TABLE 3.1
example of 25 mg film coated tablet

| | Amount per one FCT | |
|---|---|---|
| Component name | [mg/FCT] | Function |
| LOU064 | 25.000 | Drug substance |
| Copovidone | 4.000 | Stabilizer, Binder |
| Sodium lauryl sulfate | 0.100 | Stabilizer, |
| | | Surfactant |
| Water, purified | 70.900 | Vehicle |
| Wet media milled | 100.000 | |
| suspension weight | | |
| Copovidone | 8.500 | Stabilizer, Binder |
| Sodium lauryl sulfate | 0.900 | Stabilizer, |
| | | Surfactant |
| Water, purified | 22.179 | Vehicle |
| Matrix solution weight | 31.579 | |
| Suspension for Spray | 131.579 | |
| granulation amount | | |
| Mannitol SD 200 | 39.500 | Carrier |
| Dried raw granule amount | 78.000 | |
| Cellulose microcrystalline | 21.450 | Diluent |
| PH102 | | |
| Mannitol SD 200 | 21.450 | Diluent |
| Croscarmellose sodium | 7.800 | Disintegrant |
| Sodium stearyl fumarate | 1.300 | Lubricant |
| Tablet core amount | 130.000 | |
| OPADRY II WHITE | 5.554 | Coating agent |
| 85F48105.001 | | |
| OPADRY II YELLOW | 0.484 | Coating agent |
| 85F220148.001 | | |
| OPADRY II RED | 0.012 | Coating agent |
| 85F250061.001 | | |
| Water, purified | 34.283 | Vehicle |
| Amount of coating | 40.333 | |
| suspension | | |
| Film-coated tablet weight | 136.050 | |

Pharmaceutical compositions, e.g. 10 mg, 50 mg and 100 mg film coated tablet, were used in the described studies and analyzed for their REI content. Such pharmaceutical compositions have also been described in example 8 of WO/2022/162513 (attorney docket number PAT059011).

### 2. List of Equipment Used During Manufacturing

TABLE 3.2

| | | Type of | SUPAC equipment |
|---|---|---|---|
| No. | Step | equipment | class/sub-class |
| 1, 2, | Preparation of raw | Stirrer and | Convection Mixer, Low |
| 3 | suspension prior wet | vessel or | Shear/Impeller |
| | media milling | drum | |
| 4 | Wet media milling | Stirred wet | Media mill/agitated |
| | | media mill | ball mill^{a} |
| 5, 6 | Preparation of | Stirrer and | Convection Mixer, Low |
| | matrix solution | vessel | Shear/Impeller |
| 7 | Suspension for spray | Stirrer and | Convection Mixer, Low |
| | granulation | vessel | Shear/Impeller |
| 8 | Spray granulation | Stirrer and | Convection mixer, Low |
| | | vessel | Shear, Impeller, |
| | | | Jacketed |
| | | Fluidized bed | Fluid bed granulator |
| | | granulator | |
| 9 | Screening/Sieving | Screen sieve | screening mill, |
| | | | oscillating bar |
| 10 | Blending | Blender | Diffusion mixer, Bin |
| | | | blender |
| 11 | Tableting | Tablet press | Tablet Press, Power |
| | | | Assisted |
| 12 | Preparation of | Mixing: | Convection mixer, |
| | coating suspension | Stirrer and | High Shear |
| | | vessel | Rotor Stator |
| 13 | Coating | Pan coater | Perforated coating |
| | | | system |

3. Wet Milling Process (Equipment and Depiction of Wet Milling Process with Vessel in Recirculation Mode (FIG. 1)

TABLE 3.3
wet milling process, example of equipment parameter

| | | Netzsch Feinmahltechnik |
|---|---|---|
| Equipment parameters | Unit | GmbH, LabStar LS1 |
| Grinding chamber volume | mL | 600 |
| Grinding chamber cylinder | n/a | Silicium carbide (SIC) |
| liner material | | |
| Grinding chamber face side | n/a | Zirconia (Yttrium stabilized) |
| material | | |
| Rotor material in grinding | n/a | Zirconia (Yttrium stabilized) |
| chamber | | |
| Sieve cartridge type, mesh | n/a | Type woven or slotted; mesh |
| size | | size 45, or 100 μm, depending |
| | | on grinding media size |
| Grinding media vendor, | n/a | Tosoh Europe B.V., YTZ ®, |
| material | | Zirconia (Yttrium stabilized); |
| Grinding media size | μm | 100 or 200 |
| Grinding media fill level | % v/v | 80 |
| Pump type | n/a | Peristaltic pump |
| Stirred vessel volume, stirrer | n/a | Stainless steel vessel 5000 ml; |
| type | | overhead stirred |

### 4. Wet Media Milling Process Parameters Considerations

The purpose of the wet media milling process is to reduce drug substance particle size using mechanical energy input into the suspension by the relative movement of the grinding media in the grinding chamber. In general, the process can be viewed from a particle size reduction and energy point of view.

The impact of equipment and process parameters on the particle size reduction using wet media milling technology can be described by the well accepted mechanistic process model from Kwade, see Kwade (1999), Kwade (2003) and Kwade and Schwedes (2007), according to the following equation,

$${E}_{M} = SE \cdot SN \qquad ( 7 - 1 )$$

where E_{M} is the specific energy, SE is the stress energy of the grinding media and SN is the total stress number. The specific energy E_{M} can be calculated according to the following discrete equation,

$${E}_{M , i} = {\sum }_{i = 0}^{i = j} {E}_{M , i - 1} + \frac{( {P}_{i} - {P}_{0 , i} ) \cdot ( {t}_{i} - {t}_{i - 1} )}{M} \qquad ( 7 - 2 )$$

where i represents the index of summation, i=0 is the lower bound of summation representing the index at start of milling duration, i=j is the upper bound of summation representing the index at total milling duration, E_{M,i-1} is the specific energy calculated at milling duration with index i-1, Pᵢ is the power draw of the rotor of the wet media mill at milling duration with index i, P_{0,i} is the no load power draw of the rotor of the wet media mill at milling duration with index i, M is the total mass of the suspension, tᵢ is the milling duration at index i and tᵢ₋₁ is the milling duration at index i-1. The stress energy can be calculated according to Kwade by the following equation,

$$SE = {ρ}_{GM} \cdot {d}_{G M}^{3} \cdot {v}_{t}^{2} \qquad ( 7 - 3 )$$

where ρ_{GM} is density of the grinding media, d_{GM} is the diameter of the grinding media and vₜ is the tip speed of the rotor of the wet media mill. The stress energy is an almost constant parameter for the manufacturing process under development. Therefore, the average stress energy S̅E̅_{i→j} is introduced in the following discrete equation,

$${\overline{SE}}_{i \to j} = {ρ}_{GM} \cdot {d}_{GM}^{3} \cdot \frac{1}{j} \cdot {\sum }_{i = 0}^{i = j} {v}_{{t}_{t , i}}^{2} \qquad ( 7 - 4 )$$

where i represents the index of averaging, i=0 is the lower bound of averaging representing the index at start of milling duration, i=j is the upper bound of averaging representing the index at total milling duration. The stress number SN is proportional according to Kwade to the following equation for wet media mills operated in re-circulation mode,

$$SN ∝ n \cdot {N}_{GM} \cdot T \qquad ( 7 - 5 )$$

where n is the speed of the rotor of the wet media mill, N_{GM} is the number of grinding media in the grinding chamber and T is the ideal residence time of the suspension in the grinding chamber. The parameters n and N_{GM} are similar or approximately constant for manufacturing process under development. Therefore, parameter ideal residence time T can be described as the normalized turnover number N for the wet media milling operation in recirculation mode and can be calculated according to the following discrete equation,

$${N}_{i} = {\sum }_{i = 0}^{i = j} \frac{{m}_{i - 1} + {\dot{m}}_{i} \cdot ( {t}_{i} - {t}_{i - 1} )}{M} \qquad ( 7 - 6 )$$

where i represents the index of summation, i=0 is the lower bound of summation representing the index at start of milling duration, i=j is the upper bound of summation representing the index at total milling duration, mᵢ₋₁ is the cumulative mass of the suspension transported through the grinding chamber at milling duration with index i, \dot{m}ᵢ is the suspension mass flow at milling duration with index i, M is the total mass of the suspension, tᵢ is the milling duration at index i and tᵢ₋₁ is the milling duration at index i-1. Finally, the initial equation for the mechanistic process model according to Kwade can be expressed by following equation, where it is applicable for the wet media milling process in recirculation mode under development.

The study examined how the energy that is applied to the product in terms of mechanical and thermal stress during the wet media milling process influences the amount of by-product formation, as it was observed that the impurities REI increase in this process. The equipment parameters grinding media size, and the process parameters rotor tip speed (and thus also average stress energy SE) and suspension temperature were tested in different experimental designs to identify and appropriately choose the related parameters and establish proven acceptable ranges.

### Example 4: Film Coated Tablets as Exemplified in the Process Described in Wo/2022/162513

Specific batches made according to the disclosure of WO/2022/162513, for use in clinical trials, did not meet the health authority commercial requirements (i.e. did not ensure that the quality of the output material with respect to content of epoxide impurities (e.g. REI content below 30 ppm for 25 mg film coated tablet, or REI content below 7.5 ppm for 100 mg film coated tablet)). Particularly, the former process described in PAT059011 used specific energy above 2000 Kj/Kg in order to obtain desired particle size (e.g. less than 200 nm).

### Example 5: Influence of Specific Energy on Rei Content and Particle Size

TABLE 5.1
provides an overview of the executed
experimental process parameters

| Process parameter | NU4839 | NU4844 | NU4847 |
|---|---|---|---|
| Batch size/kg | 175 | 175 | 175 |
| Specific energy (E_{M})*^{,+}/kJ/kg | 1400 | 1400 | 2400 |
| Average stress energy (SE) μNm | 9.1 | 9.1 | 9.1 |
| Turnover number (N)*^{,+}/— | 70 | 70 | 116 |
| Suspension outlet temperature | 21-24 | 21-23 | 21-23 |
| (T)^{#}/° C. | | | |
| (measured, range of resulting | | | |
| parameter) | | | |
| Rotor speed/rpm (given parameter) | 1050 | 1050 | 1050 |
| Rotor tip speed (vt)^{#}/m/s | 12 | 12 | 12 |
| (calculated, resulting parameter) | | | |
| Volume flow (V)^{#}/l/min (measured, | 11-16 | 9-16 | 10-16 |
| range of resulting parameter | | | |
| including ramp up | | | |

⁺The variables specific energy (E_{M}) and turnover number (N) are evolving parameters with associated in-process sampling, and were therefore investigated in a range per experimental trial.
*The process is operated up to the target specific energy (E_{M}) as end point variable per experimental trial; process parameter turnover number (N) is a resulting parameter and not considered to be operated to a certain target.
^{#}Process parameter Rotor tip speed (vₜ), Volume flow (V), turnover number (N) and suspension outlet temperature (T) are resulting parameter and not considered to be operated at or to a certain target.

All batches were manufactured according to the wet milling process described in example 3, without any processability issues.

The suspension samples from the experimental trials were analyzed for REI content. The quantification of REI in the suspension is performed using High-Pressure Liquid Chromatography (HPLC) coupled to a single quadrupole mass spectrometer. 5.1 REI content versus applied specific energy Tables 5.2 to 5.4 summarize the process parameters specific energy (E_{M}), turnover number (N), average stress energy (S̅E̅) and suspension outlet temperature (T) and the corresponding analytical data for average particle size (xPCS), particle size distribution by polydispersity index (PI) and specific impurities REI for each batch.

TABLE 5.2
Experimental results for quantitative particle size, REI content of batch NU4839

| | | | Average | Suspension | Average | | |
|---|---|---|---|---|---|---|---|
| | Specific | | stress | outlet | particle | Poly- | |
| | energy | Turnover | energy | temperature | size | dispersity | |
| Sample | (E_{M})/ | number | (S̅E̅)/ | (T)/ | (X_{PCS})/ | index | REI |
| description | kJ/kg | (N)/- | μNm | ° C. | nm | (PI)/- | (ppm) |
| Drug | n.a | n.a. | n.a. | n.a. | n.a. | n.a. | 15 |
| substance | | | | | | | |
| Before start | n.a | n.a. | n.a. | n.a. | n.a. | n.a. | 21 |
| of milling | | | | | | | |
| at 150 | 150 | 5 | 9.09 | 24 | 283 | 0.19 | — |
| KJ/kg | | | | | | | |
| at 400 | 400 | 17 | 9.08 | 24 | 205 | 0.12 | 34 |
| KJ/kg | | | | | | | |
| at 650 | 650 | 28 | 9.07 | 24 | 178 | 0.12 | 41 |
| KJ/kg | | | | | | | |
| at 900 | 900 | 43 | 9.07 | 23 | 166 | 0.11 | 44 |
| KJ/kg | | | | | | | |
| at 1150 | 1150 | 57 | 9.07 | 23 | 158 | 0.12 | — |
| KJ/kg | | | | | | | |
| at 1400 | 1400 | 70 | 9.07 | 23 | 152 | 0.12 | 50 |
| KJ/Kg | | | | | | | |

TABLE 5.3
Experimental results for quantitative particle size, REI content of batch NU4844

| | | | Average | Suspension | Average | | |
|---|---|---|---|---|---|---|---|
| | Specific | | stress | outlet | particle | Poly- | |
| | energy | Turnover | energy | temperature | size | dispersity | |
| Sample | (E_{M})/ | number | (S̅E̅)/ | (T)/ | (X_{PCS})/ | index | REI |
| description | kJ/kg | (N)/- | μNm | ° C. | nm | (PI)/- | ppm |
| Drug | n.a | n.a | n.a | n.a | n.a | n.a | 8 |
| substance | | | | | | | |
| Before start | n.a | n.a | n.a | n.a | n.a | n.a | 12 |
| of milling | | | | | | | |
| at 150 kJ/kg | 150 | 4 | 9.09 | 23 | 360 | 0.34 | 23 |
| at 400 kJ/kg | 400 | 17 | 9.08 | 22 | 251 | 0.26 | 27 |
| at 650 | 650 | 29 | 9.07 | 22 | 182 | 0.12 | 35 |
| kJ/kg | | | | | | | |
| at 900 kJ/kg | 900 | 42 | 9.07 | 22 | 168 | 0.12 | 34 |
| at 1150 | 1150 | 56 | 9.07 | 22 | 162 | 0.11 | 36 |
| kJ/kg | | | | | | | |
| at 1400 | 1400 | 70 | 9.07 | 21 | 154 | 0.11 | 38 |
| kJ/kg | | | | | | | |

TABLE 5.4
Experimental results for quantitative particle size, REI content in batch NU4847

| | | | Average | Suspension | Average | | |
|---|---|---|---|---|---|---|---|
| | Specific | | stress | outlet | particle | Poly- | |
| | energy | Turnover | energy | temperature | size | dispersity | |
| Sample | (EM)/ | number | (S̅E̅)/ | (T)/ | (xPCS)/ | index | REI |
| description | kJ/kg | (N)/- | μNm | ° C. | nm | (PI)/- | ppm |
| Drug | n.a | n.a | n.a | n.a | n.a | n.a | 9 |
| substance | | | | | | | |
| Before start | n.a | n.a | n.a | n.a | n.a | n.a | 9 |
| of milling | | | | | | | |
| at 150 kJ/kg | 150 | 5 | 9.09 | 23 | 289 | 0.2 | 20 |
| at 400 kJ/kg | 400 | 17 | 9.08 | 22 | 210 | 0.13 | 26 |
| at 650 kJ/kg | 650 | 29 | 9.07 | 22 | 184 | 0.11 | 29 |
| at 900 kJ/kg | 900 | 42 | 9.07 | 22 | 170 | 0.1 | 27 |
| at 1150 | 1150 | 55 | 9.07 | 22 | 165 | 0.15 | 35 |
| kJ/kg | | | | | | | |
| at 1400 | 1400 | 67 | 9.07 | 22 | 156 | 0.11 | 37 |
| kJ/kg | | | | | | | |
| at 1650 | 1650 | 81 | 9.07 | 21 | 150 | 0.121 | — |
| kJ/kg | | | | | | | |
| at 1900 | 1900 | 93 | 9.07 | 21 | 147 | 0.11 | 43 |
| kJ/kg | | | | | | | |
| at 2150 | 2150 | 105 | 9.07 | 21 | 144 | 0.13 | — |
| kJ/kg | | | | | | | |
| at 2400 | 2400 | 118 | 9.07 | 21 | 144 | 0.1 | 44 |
| kJ/kg | | | | | | | |

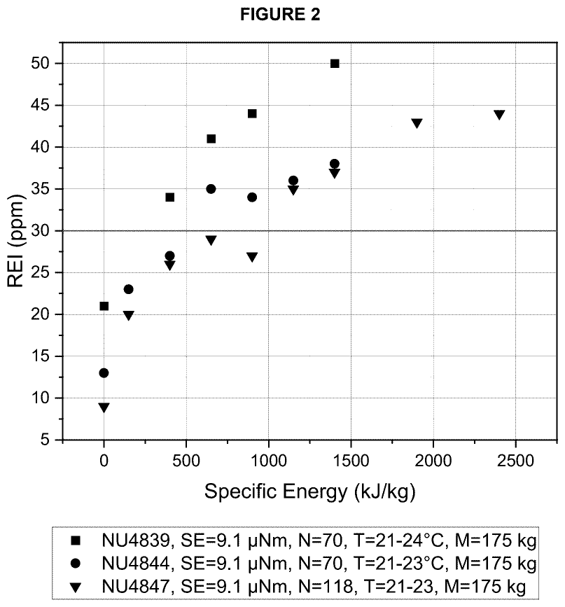

### FIG. 2 Represents REI Content Versus Applied Specific Energy (E_{M})

FIG. 2 shows that REI level increases as the specific energy does and exceeds the commercial specifications limit of 30 ppm (for 25 mg) already after a specific energy of about 650 KJ/Kg.

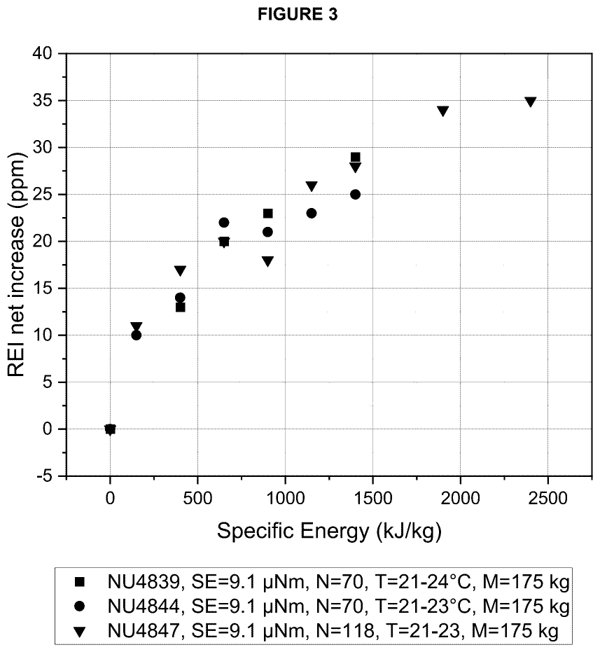

The different batches had different REI levels in the raw suspension which is driven by the respective used drug substance batch. To allow for better comparability, the net increase of the REI concentration was calculated by subtracting the value of the raw suspension from the measured values. Results are shown in FIG. 3. (REI net increase versus applied specific energy).

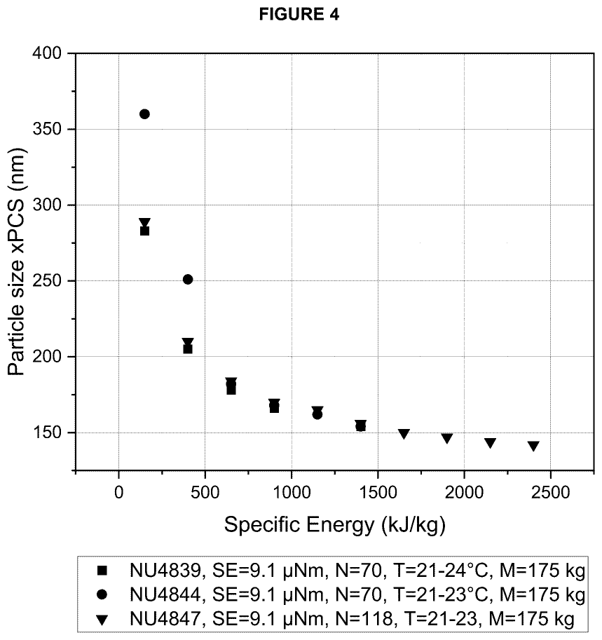

5.2 Obtained Particle Size Versus Applied Specific Energy The obtained average particle size results (x_{PCS}) against process parameter specific energy (E_{M}) including applied average stress energy (S̅E̅), turnover number (N), suspension outlet temperature (T) and batch size (M) is displayed in FIG. 4.

FIG. 4 shows that Specific energy (E_{M}) is an important process parameter related to the reduction of particle size.

It was determined that specific energy (E_{M}) applied during the milling process is the key factor, impacting drug substance particle size in the suspension. Particle size decreases with increasing specific energy. It also impacts the formation of impurity REI which constantly increases with specific energy.

### Example 6: Influence of Grinding Media Size/Average Stress Energy (Se) on Rei Net Increase

The data obtained in example 5.2 above, were repeated using 100 um grinding beads in the wet milling process.

TABLE 6.1
Overview of executed process parameters of manufactured
batches NV9429, NV8252 and NV5238:

| Process parameter | NV5238 | NV8252 | NV9439 |
|---|---|---|---|
| Batch size/kg | 60 | 60 | 60 |
| Specific energy (E_{M})*^{,+}/kJ/kg | 2400 | 1400 | 1400 |
| Average stress energy (SE) μNm | 0.87 | 0.87 | 0.61 |
| Turnover number (N)*^{,+}/— | 182 | 103 | 153 |
| Suspension outlet temperature | 19-22 | 19-22 | 15-17 |
| (T)^{#}/° C. | | | |
| (measured, range of resulting | 1050 | 1050 | 875 |
| parameter) | | | |
| Rotor speed/rpm (given parameter) | | | |
| Rotor tip speed (vt)^{#}/m/s | 12 | 12 | 10 |
| (calculated, resulting parameter) | | | |
| Volume flow (V)^{#}/l/min (measured, | 8-16 | 8-17 | 6-18 |
| range of resulting parameter | | | |
| including ramp up | | | |

⁺The variables specific energy (E_{M}) and turnover number (N) are evolving parameters with associated in-process sampling, and were therefore investigated in a range per experimental trial.
*The process is operated up to the target specific energy (E_{M}) as end point variable per experimental trial; process parameter turnover number (N) is a resulting parameter and not considered to be operated to a certain target.
^{#}Process parameter Rotor tip speed (vₜ), Volume flow (V), turnover number (N) and suspension outlet temperature (T) are resulting parameter and not considered to be operated at or to a certain target.

TABLE 6.2
REI net increase, comparison between
100 and 200 μm grinding media size

| | Grinding | | | |
|---|---|---|---|---|
| | media | size REI at | REI at | REI net |
| Batch | μm | 0 KJ/kg/ppm | 1400 KJ/kg/ppm | increase/ppm |
| NV9429 | 100 | 3 | 13 | 10 |
| NV8252 | 100 | 0 | 14 | 14 |
| NV5238 | 100 | 1 | 9 | 8 |
| NU4847 | 200 | 9 | 27 | 36 |
| NU4846 | 200 | 8 | 19 | 11 |
| NU4844 | 200 | 13 | 35 | 27 |
| NU4839 | 200 | 21 | 45 | 30 |

REI net increase is two to four times higher, when using 200 μm grinding media

Overall, the net increase of REI using 200 um beads is higher compared to the REI content using 100 um beads. This indicates that high average stress energies also promote formation of REI and that it is advantageous to use 100 nm beads for the wet milling process.

### Example 7: Influence of Length of Milling on Rei Content and Particle Size

TABLE 7
Overview of process parameters

| | Drug | | | | |
|---|---|---|---|---|---|
| | Substance | | Suspension | | |
| | REI | Tip | outlet | Grinding | Nitro- |
| | content | speed/ | temperature/ | media | gen |
| Batch | (ppm) | m/s | ° C. | size/μm | purge |
| LOU064-# 100 | 1 | 12 | 20 | 100 | Yes |
| LOU064-#-101 | 1 | 10 | 14 | 100 | Yes |
| LOU064-# 102 | 1 | 12 | 25 | 100 | No |
| LOU064-# 103 | 1 | 10 | 18 | 200 | Yes |
| LOU064-# 106 | 1 | 10 | 12-16 | 100 | Yes |
| LOU064 # 107 | 1 | 12 | 22-25 | 100 | No |

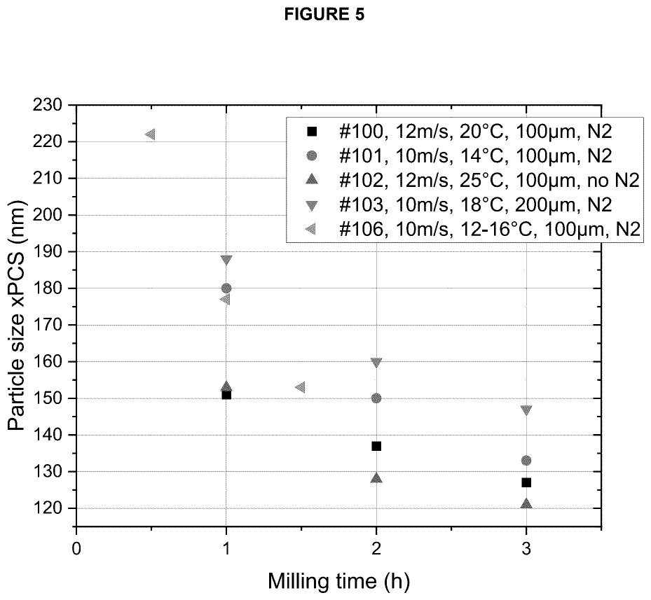

FIG. 5 Shows the Development of Particle Size xPCS Over Milling Time:

The particle size decreases by increasing the milling time for all batches (see FIG. 5). By reducing the tip speed from 12 to 10 m/s, coarser particles are obtained after the same milling time (compare batches #101 and #106 to batches #100 and #102). By increasing the bead size to 200 pm (batch #103), the obtained particle size at a specific milling time is even higher due to the reduced number of stress events but still below the desired limit of 200 nm already after 1 hour of milling.

Batch #100 was manufactured at a lower suspension outlet temperature compared to batch #102. To achieve this at a tip speed of 12 m/s, the temperature of the cooling liquid used to cool the mill and stirred vessel had to be reduced from −20° C. to −35° C. As a consequence, part of the suspension froze in the stirred vessel which might contribute to the fact that coarser particles were measured compared to batch #102 at the same milling time.

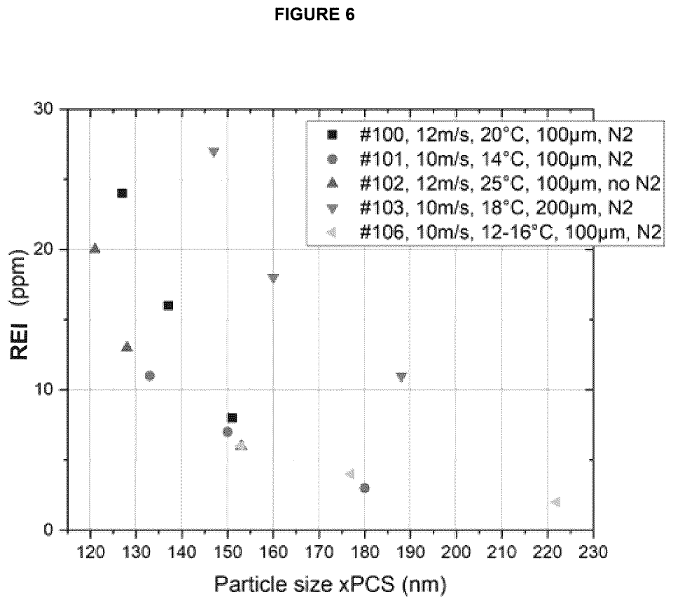

FIG. 6 Shows the Development of Impurity REI Over Particle Size xPCS

For all batches under investigation, the level of impurity REI increases when the milling time increases and, hence, particle size is decreased (see FIG. 6). Batch #102 was run under standard process conditions in terms of tip speed, suspension outlet temperature and grinding media size. Batch #101 was manufactured by using a lower tip speed and a lower suspension outlet temperature. Additionally, nitrogen flush has been applied for the stirred vessel. By comparing the obtained data, no obvious differences can be observed regarding the obtained REI level for a certain product particle size.

According to the Kwade model the parameters tip speed and grinding media size (and density) affect the average stress energy which can be calculated according to

$$SE = {ρ}_{GM} \cdot {d}_{G M}^{3} \cdot {v}_{t}^{2} \qquad ( 4 - 1 )$$

where ρ_{GM} is density of the grinding media, d_{GM} is the diameter of the grinding media and vₜ is the tip speed of the rotor of the wet media mill. Taking nominal values from the grinding media bead specification (Size: 100 μm, Density 5.95 g/cm³) this translates into an average stress energy of 0.86 pNm for batch #102 and an average stress energy of 0.6 pNm for batch #101. Reducing average stress energy about 30% does not seem to impact REI formation.

However, batch #103 was manufactured using 200 μm beads at a tip speed of 10 m/s which translates into an average stress energy of 4.76 μNm. The obtained values for REI for a specific product particle size is significantly higher compared to the other experiments which indicates that by significantly increasing the average stress energy by a factor of 5.4 compared to the standard experiment (batch #102), the impurity formation is also significantly increased.

As indicated above, batch #100 was using also standard parameters apart from a reduced suspension outlet temperature. As described, the particle size for a specific milling time was coarser for this batch compared to batch #102 due to freezing of the product in the stirred vessel. As the rest of the parameters (in particular bead size and tip speed) were the same it can be assumed that the transferred mechanical energy to the suspension was still similar. This could explain why for this experiment at a certain specific particle size the REI content seems to be higher compared to experiments #101 and #102 when plotted against particle size.

Based on the graph, stress energy and specific energy are main drivers for impurity REI formation.

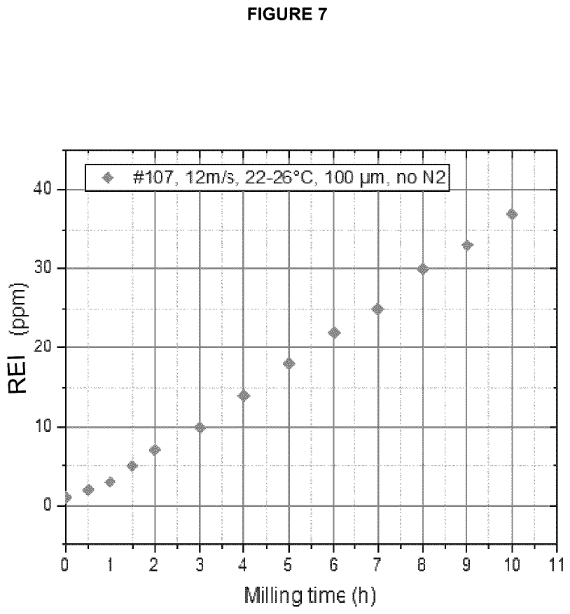

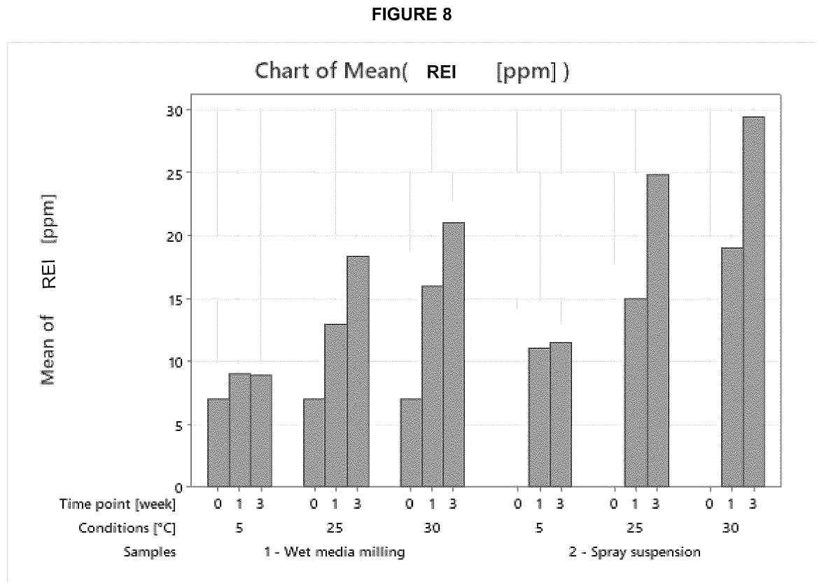

In order to understand if there was a limit to REI formation during the milling process, a long-term milling experiment was conducted (LOU064 #0107). The results are displayed in FIG. 7.

### FIG. 7 Shows the REI Content Versus the Milling Time.

The REI content is increasing almost linearly with milling time and does not reach any plateau within the investigated time frame of 10 hours. Thus, as shown before, limiting the milling time (or specific energy input, respectively) is key to control the formation of REI during the wet media milling process.

### Example 8: Influence of Holding Time (Wet Milling Suspension & Spray Granulation Suspension) On REI Content

Holding time of wet media milling suspension and suspension for spray has been conducted where different temperatures were studied after 1 week and 3 weeks.

For this investigation, batches LOU064 #106 (nanosuspension) and suspension for spray granulation-batch ORA-0247) have been manufactured from a DS batch containing 1 ppm or REI

TABLE 8
Development of REI over time and
temperature (holding time study)

| | | | REI/ppm | | |
|---|---|---|---|---|---|
| Batch number | Time point | | 5° C. | 25° C. | 30° C. |
| Wet medial milling | T0 | 7 | — | — | — |
| suspension | 1 week (1 W) | — | 9 | 13 | 16 |
| LOU064 #-106 | 3 week (3 W) | — | 8.9 | 18.3 | 21 |
| Suspension for spray | T0 | — | — | — | — |
| granulation | 1 week (1 W) | — | 11 | 15 | 19 |
| LOU064-ORA-0247 | 3 week (3 W) | — | 11.5 | 24.8 | 29.4 |

Figure shows the development of REI over time and temperature.

The starting value for REI of the drug substance is 1 ppm. Investigations has concluded that specific energy applied during the milling process is the key factor, impacting DS particle size in the suspension and it also impacts the formation of REI impurity. REI levels rise from 1 ppm (REI level in DS) to 7 ppm at the end of the milling step (TO).

After 1 and 3 weeks storage of the wet media milled suspension at 5° C., an increase of 2-3 ppm is observed. After only 1 week storage at higher temperature (25° C. and 30° C.), a higher increase of 5 to 11 ppm is measured.

For spray suspension there is no REI value measured at TO. Same increasing trend is observed when storage at higher temperatures and over time.

The holding time study of wet media milling suspension and spray suspension indicates that REI levels increase with high temperature (25° C. and 30° C.) over time. Storage in a cooler condition is beneficial to maintain the REI content in the product suspension over time.

It could also be concluded that using lower outlet temperature is beneficial to reduce the REI content during wet milling process.

### Example 9A: Influence of Excipient (E.G. Copovidone) Peroxide Content on Rei Content

As peroxides could facilitate the formation of REI, an additional experiment has been conducted where Copovidone from a different supplier (Ashland) was used with low level of peroxides. According to the CoA, Ashland copovidone has a peroxide content of 7 ppm whereas the material from BASF has <30 ppm peroxide content.

LOU064 #104 was manufactured with a copovidone grade from Ashland that has a lower peroxide content compared to the quality from BASF which has been the standard used in manufacturing process. Purpose of the experiment was to identify if a reduction in peroxide in the excipient can reduce the amount of REI which is created during the milling process.

TABLE 9.1
Overview of process parameters

| | Drug | | Suspen- | | | |
|---|---|---|---|---|---|---|
| | sub- | | sion | | | |
| | stance | | outlet | Grinding | | |
| | REI | Tip | temper- | media | Nitro- | Copovi- |
| | content | speed/ | ature/ | size/ | gen | done |
| Batch | (ppm) | m/s | ° C. | μm | purge | supplier |
| LOU064- | 5 | 12 | 32 | 100 | Yes | BASF |
| # 098 | | | | | | |
| LOU064 | 7 | 12 | 26 | 100 | No | BASF |
| # 099 | | | | | | |
| LOU064 | 1 | 12 | 25 | 100 | No | BASF |
| # 102 | | | | | | |
| LOU064 | 1 | 12 | 25 | 100 | No | Ashland |
| # 104 | | | | | | |

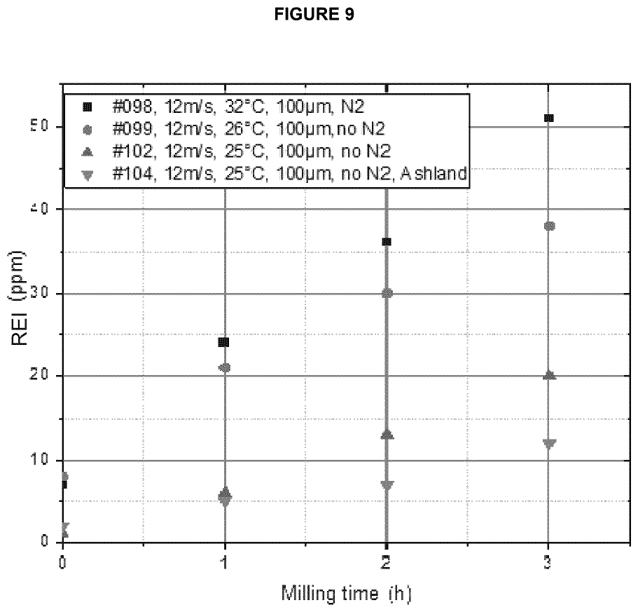

FIG. 9 shows the development of impurity REI over milling time for different drug substance batches and copovidone supply

Batches #099 and #102 were manufactured under the same process conditions during wet media milling. Only difference was the drug substance (DS) batch which was used which already showed an elevated level of REI. By comparing the obtained results for impurity REI for batch #099 with those for batch #102 it was seen that the DS quality impacts the quantity of impurity formation during the wet media milling process (see FIG. 9). Batch #98 was manufactured at a slightly higher temperature during the wet media milling process and by applying nitrogen flush in the stirred vessel. It shows the overall highest level of REI which was observed in all experiments so far. As mentioned in Example 8, the results of this experiment also suggest that using lower outlet temperature is beneficial to reduce the REI content during wet milling process.

Batch #104 was manufactured using the same DS and the same process parameters compared to batch #102. Only difference was that the used copovidone to prepare the raw suspension had a lower peroxide content. Although the starting value for REI of the raw suspension is slightly higher for batch #104 than for batch #102, the obtained results for batch #104 throughout the process are constantly below those of batch #102. This indicates that a reduced peroxide level in the copovidone is beneficial to reduce the REI content in the product suspension.

### Example 9B: Investigation of Peroxide Level in Additional Excipients

Peroxide levels were evaluated in the excipients and the results are provided in Table 9b.

For cellulose, croscarmellose sodium, sodium lauryl sulfate and mannitol the results were below 10 ppm. Only the lubricant exhibited increased levels which were below 20 ppm.

TABLE 9b
Peroxides value in excipients

| | Batch | Peroxide value as |
|---|---|---|
| Excipient | number | H₂O₂ [ppm] |
| Cellulose MK GR | S011902 | <10 |
| Microcrystalline Cellulose PH102 LN | S0001 | <10 |
| Crosscarmellose sodium | S0024 | <10 |
| Sodium lauryl sulfate | S0007 | <10 |
| Mannitol | S0028 | <10 |
| Sodium stearyl fumarate | S0020 | <20 |

Copovidone has therefore been identified as the excipient with a significant potential source of reactive peroxide which can react with remibrutinib to produce REI. Given copovidone accounts for approximately 9.6% w/w of remibrutinib composition (and about 4% in the wet media milling suspension), it is important to use copovidone with low level of peroxide content (e.g. less than 30 ppm, less than 20 ppm and preferably less than 10 ppm).

CONCLUSION: Ranges Selected—Optimizing the Particle Size (Less than or Equal to 200 nm) and REI Content (Up to 30 ppm, Preferably Up to 25 ppm, Up to 20 ppm)

Based on the above experiment in order to optimize the wet milling process while keeping the particle size (up to 200 nm) and minimizing the REI content (no more than 30 ppm, no more than 25 ppm, no more than 20 ppm, no more than 15 ppm or no more than 7.5 ppm, or no more than 5 ppm)). Parameters which were determined relevant as determined by experiments described in examples 4-x) were:
1. Suspension outlet temperature in grinding chamber
2. Specific energy
3. Average stress energy
4. Turnover number

In order to meet all desired requirements (particle size and REI content), the following acceptable ranges were determined based on above experiments:

| Variable description | Proven Acceptable Ranges* |
|---|---|
| Suspension outlet | Not more than 20° C. |
| temperature in grinding | |
| chamber | |
| Specific energy | 800 kJ/kg [600-1000] |
| Average stress energy | Not more than 1.3 μNm |
| (via reduction of | |
| grinding media size) | |
| Turnover number | [28-106] |
| Wet media milling | ≤200 nm |
| suspension DS particle | |
| size | |

### Influence of Turnover Number on REI Content

Turnover number is a resulting parameter and evolves throughout the grinding process. It is calculated taking the batch size, the mass flow and the milling duration into consideration. Milling time depends on the specific energy which has been varied within a broad range as described earlier. The TN range was selected on actual process data (either as in process control (IPC) or end point) when the following criteria were met: 1. Specific energy was in the defined process acceptable range (PAR), 2. REI and particle size data was within the specifications.

### Example 10: Validation of the Above Selected Ranges (Par: Process Acceptable Range)

Further experiments were carried out within the above determined ranges, to verify that all batches manufactured using the above selected parameters and ranges (TN, E_{M} and SE) provided desired particle size and distribution as well a desired content of REI (up to 20 ppm)

TABLE 10.1
Overview of executed parameters of manufactured batches

| | Batch | | |
|---|---|---|---|
| | (−) | | (+) |
| | level | Setpoint | level |
| Process parameter | NX0912 | NW4732 | NX0913 |
| Batch size/kg | 50.0 | 155.0 | 50.0 |
| Specific energy (E_{M})*^{, +}/kJ/kg | 600 | 800 | 1000 |
| Average stress energy (S̅E̅_{Actual}) | 0.67 | 1.00 | 1.32 |
| μNm, recalculated | | | |
| Turnover number (N)*^{, +}/− | 63 | 55 | 50 |
| Suspension outlet temperature (T)^{#}/° | 12-13 | 14-15 | 22-26 |
| C. (measured, range of resulting | | | |
| parameter) | | | |
| Rotor speed/rpm (given parameter) | 875 | 1050 | 1225 |
| Rotor tip speed (vt)^{#}/m/s (calculated, | 10 | 12 | 14 |
| resulting parameter) | | | |
| Volume flow (\dot{V})^{#}/l/min (measured, | 8-16 | 10-16 | 9-16 |
| range of resulting parameter | | | |
| including ramp up) | | | |
| External heat exchanger | Yes | Yes | No |

TABLE 10.2
Grinding beads used for DP manufacturing

| DP Batch | Bead batch | Average bead | Bead density (ρ)/ |
|---|---|---|---|
| number | number | diameter (d)/μm | kg/m³ |
| NW4732 | 32488846 | 107 | 6070 |
| NX0912 | 32534764 | 105 | 6070 |
| NX0913 | 32534764 | 105 | 6070 |

TABLE 10.3
Experimental results for quantitative particle size. REI content of batch NVP4732 (Setpoint)

| | | | Recalculated | | | | |
|---|---|---|---|---|---|---|---|
| | | Average | average | Suspension | Average | | |
| | | stress | stress | outlet | particle | Poly- | |
| | Turnover | energy | energy | temperature | size | dispersity | |
| | number | (S̅E̅_{Recipe})/ | (S̅E̅_{Actual})/ | (T)/ | (X_{PCS})/ | index | REI |
| Sample | (N)/- | μNm | μNm | ° C. | nm | (PI) | ppm |
| LOU064- | n.a. | n.a. | n.a. | n.a. | n.a. | n.a. | 1 |
| DS | | | | | | | |
| batch | | | | | | | |
| Before | n.a. | n.a. | n.a. | n.a. | n.a. | n.a. | 1.5 |
| start of | | | | | | | |
| milling | | | | | | | |
| at 150 | 9 | 0.88 | 1.01 | 14 | 268 | 0.131 | 3.5 |
| kJ/kg | | | | | | | |
| at 300 | 20 | 0.88 | 1.01 | 14 | 204 | 0.098 | 3.4 |
| kJ/kg | | | | | | | |
| at 450 | 31 | 0.88 | 1.01 | 15 | 177 | 0.108 | 3.6 |
| kJ/kg | | | | | | | |
| at 600 | 41 | 0.87 | 1.00 | 15 | 166 | 0.111 | 3.6 |
| kJ/kg | | | | | | | |
| at 700 | 48 | 0.87 | 1.00 | 15 | 161 | 0.107 | 3.8 |
| kJ/kg | | | | | | | |
| at 800 | 55 | 0.87 | 1.00 | 15 | 156 | 0.110 | 3.7 |
| kJ/kg | | | | | | | |

TABLE 10.4
Experimental results for quantitative particle size, REI content of batch NVP0913 (+ level)

| | | | Recalculated | | | | |
|---|---|---|---|---|---|---|---|
| | | Average | average | Suspension | Average | | |
| | | stress | stress | outlet | particle | Poly- | |
| | Turnover | energy | energy | temperature | size | dispersity | |
| | number | (S̅E̅_{Recipe})/ | (S̅E̅_{Actual})/ | (T)/ | (X_{PCS})/ | index | REI/ |
| Sample | (N)/- | μNm | μNm | ° C. | nm | (PI)/- | ppm |
| LOU064- | n.a. | n.a. | n.a. | n.a. | n.a. | n.a. | 1 |
| DS | | | | | | | |
| Before | n.a. | n.a. | n.a. | n.a. | n.a. | n.a. | 2.7 |
| start of | | | | | | | |
| milling | | | | | | | |
| at 150 | 4 | 1.19 | 1.32 | 25 | 249 | 0.159 | 5.4 |
| kJ/kg | | | | | | | |
| at 300 | 12 | 1.19 | 1.32 | 26 | 192 | 0.097 | 7.5 |
| kJ/kg | | | | | | | |
| at 450 | 19 | 1.19 | 1.32 | 26 | 171 | 0.117 | 9.5 |
| kJ/kg | | | | | | | |
| at 600 | 28 | 1.19 | 1.32 | 26 | 159 | 0.095 | 11.4 |
| kJ/kg | | | | | | | |
| at 700 | 33 | 1.19 | 1.32 | 26 | 151 | 0.095 | 13.0 |
| kJ/kg | | | | | | | |
| at 800 | 39 | 1.19 | 1.32 | 26 | 145 | 0.119 | 14.2 |
| kJ/kg | | | | | | | |
| at 900 | 45 | 1.19 | 1.32 | 26 | 142 | 0.099 | 15.6 |
| kJ/kg | | | | | | | |
| at 1000 | 50 | 1.19 | 1.32 | 26 | 138 | 0.122 | 16.8 |
| kJ/kg | | | | | | | |

TABLE 10.5
Experimental results for quantitative particle size, REI content of batch NVP0912 (− level)

| | | | Recalculated | | | | |
|---|---|---|---|---|---|---|---|
| | | Average | average | Suspension | Average | | |
| | | stress | stress | outlet | particle | Poly- | |
| | Turnover | energy | energy | temperature | size | dispersity | |
| | number | (S̅E̅_{Recipe})/ | (S̅E̅_{Actual})/ | (T)/ | (X_{PCS})/ | index | REI/ |
| Sample | (N)/- | μNm | μNm | ° C. | nm | (PI)/- | ppm |
| LOU064-DS | n.a. | n.a. | n.a. | n.a. | n.a. | n.a. | 1 |
| Before start | n.a. | n.a. | n.a. | n.a. | n.a. | n.a. | 2.6 |
| of milling | | | | | | | |
| at150 kJ/kg | 12 | 0.61 | 0.67 | 12 | 422 | 0.325 | 5.3 |
| at 300 kJ/kg | 30 | 0.61 | 0.67 | 12 | 223 | 0.155 | 5.8 |
| at 450 kJ/kg | 46 | 0.61 | 0.67 | 12 | 190 | 0.086 | 6.5 |
| at 600 kJ/kg | 63 | 0.61 | 0.67 | 12 | 176 | 0.095 | 6.4 |

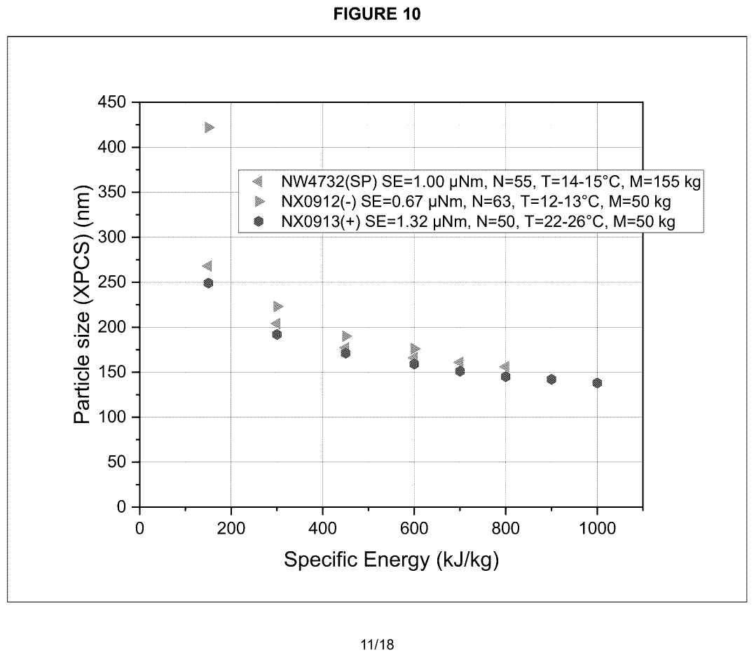

### FIG. 10 Shows the Obtained Particle Size Versus Applied Specific Energy

The obtained average particle size results (x_{PCS}) plotted against process parameter specific energy (E_{M}) including applied average stress energy (SE), turnover number at the end of the process (N), suspension outlet temperature (T) and batch size (M) are displayed in FIG. 10. Specific energy (E_{M}) is the most relevant process parameter related to the reduction of particle size. At low specific energy input (150 kJ/kg), there is a noticeable impact of parameter average stress energy on product particle size. Generally, variability of the particle size is higher at the beginning of the process due to the low turnover number and back mixing in the stirred vessel. Coarse DS particles need multiple stress events to milled down, particularly if stressed at lower average stress energies. The general results support the 600-1000 KJ/Kg range.

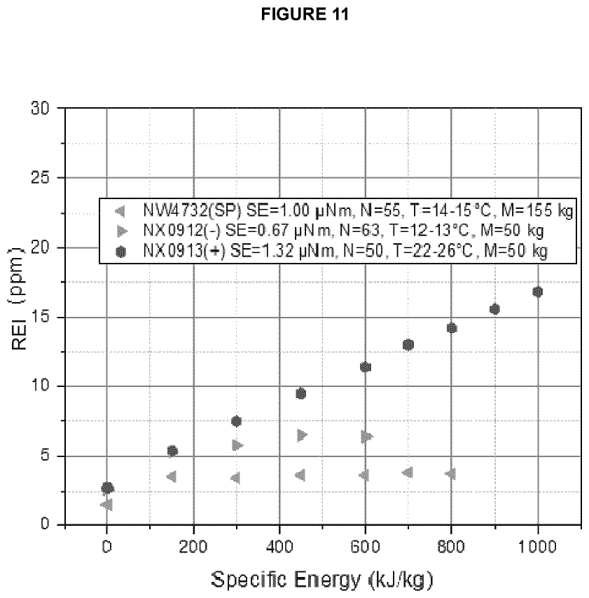

### FIG. 11 Shows the REI Content Versus Applied Specific Energy.

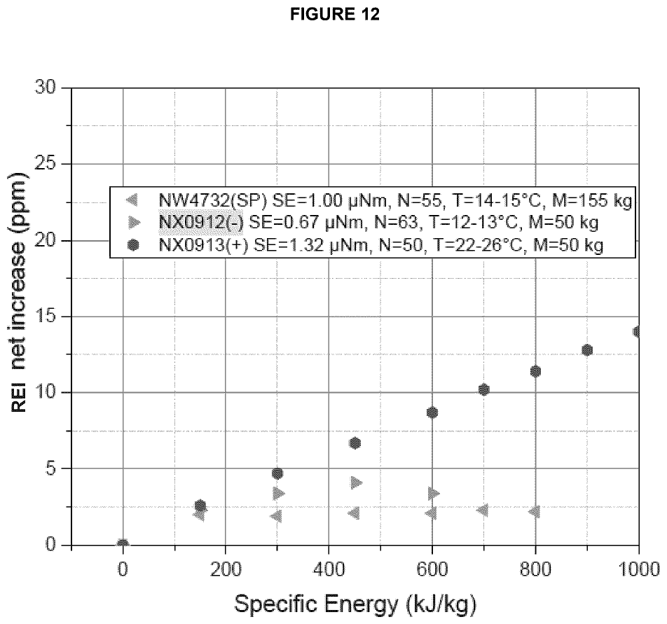

FIG. 11 shows that the REI level increases as the specific energy does at the beginning of the milling process up to 150 kJ/kg for all batches. Depending on the chosen process conditions, REI continues to increase for the minus batch NX0912 up to 500 kJ/kg and up to the end of the process for plus batch NX0913 (higher temperature, higher average stress energy). The maximum impurity level in the tested ranges is about half of the targeted commercial specification of 30 ppm. Batch NW4732 has the lowest REI level throughout the milling process. As the same drug substance batch was used for all batches, the REI level of the raw suspension is expected to be comparable for all batches. Therefore, the net REI increase during the milling process was calculated by subtracting the obtained value of the raw suspension of each measurement for the individual batches. Results are displayed in FIG. 12:

### FIG. 12: Net Increase of REI Content Versus Specific Energy

Key differences between batches NX0912 and NX0913 were the applied average stress energy and the suspension outlet temperature which were both higher for batch NX0913. Batch NX0913 shows a continuous increase of the REI content whereas batch NX0912 is seemingly reaching a plateau after about 300 kJ/kg specific energy input. By comparing batches NX0912 and NW4732 the key differences were the applied average stress energy and the batch size, which was both higher for batch NW4732. As the REI levels are comparable or even lower for the latter batch, an increase of the average stress energy of about 50% and an increase of the batch size about factor three did not negatively impact the impurity profile. When comparing Batches NW4732 and NX0913, the average stress energy is again increased about 32% while the suspension outlet temperature is almost doubled for NX0913. Which parameter is key to drive the difference in the REI formation is not fully understood, yet. From experiments described above, it was discovered that if the average stress energy is significantly increased (by using larger grinding media), REI formation is also promoted. Thus, it was decided to control average stress energy as well as suspension outlet temperature to gain proper control on the REI content, this in addition of limiting the overall applied specific energy. Within the investigated ranges (maximum specific energy=1000 kJ/kg, maximum stress energy=1.32 μNm, maximum suspension outlet temperature=26° C.) the obtained REI level is still about half of the targeted specification limit of 30 ppm (for 25 mg BID use) which is why these parameters are considered to be appropriate to control REI formation during the wet media milling process.

### Example 11: Downstream Process: Impact of Granulation, Blending and Tableting Steps on Rei Formation

The granulation step is followed by a sieving step of the granules and the outer phase excipients such as filler (microcrystalline cellulose, Mannitol SD 200), super-disintegrant (Croscarmellose sodium) and lubricant (Sodium Stearyl Fumarate). The sieved granules are blended with the sieved outer phase excipients to achieve the final blend. The LOU064 final blend is compressed into tablet cores using a power assisted tablet press. Finally, tablet cores are coated with Opadry II suspension, a non-functional immediate release film coating.

Level of REI were measured in drug substance, after wet-media milling (process as disclosed in PAT059011), in granules and in tablets.

The results do not exhibit a significant increase on the levels for REI.

TABLE 11
Laboratory scale evaluation for REI

| Drug | After wet-media | | |
|---|---|---|---|
| substance | milling | Granules | Tablets |
| 7 ppm | 38 ppm | Sample 1: 41 ppm | Sample 1: 40 ppm |
| | | Sample 2: 48 ppm | Sample 2: 39 ppm |

The increase in the REI levels occurs at the wet media milling step. For the granules and tablets, the differences might be associated to the analytical and sample variability.

This experiment allowed the optimization of the wet media milling process in order to reach lower level of REI (e.g. below 30 ppm, below 7.5 ppm) is sufficient to insensurew level of REI in the drug product.

### Example 12: Stability Results Related to Rei in Drug Product (Dp: 25 Mg Fct)

An initial Accelerated Predictive Stability (APS) assessment was conducted assessing the behavior of REI under different temperature and humidity conditions. Based on the results, both relative humidity and temperature do not appear to play a significant role in the formation of REI

TABLE 12.1
APS behavior of drug product 1 (performed under the optimized
conditions as disclosed herein) under open dish storage
at various temperature humidity conditions

| | | | REI [ppm] |
|---|---|---|---|
| Temp. (° C.) | RH(%) | Time (days) | DP 1 |
| NA | NA | 0 | 18 |
| 80 | 50 | 7 | 24 |
| 50 | 75 | 14 | 27 |
| 60 | 50 | 14 | 23 |
| 60 | 75 | 14 | 30 |
| 70 | 31 | 14 | 19 |
| 70 | 75 | 14 | 33 |
| 50 | 75 | 21 | 28 |
| 60 | 11 | 21 | 23 |
| 60 | 75 | 21 | 31 |
| 70 | 50 | 21 | 22 |
| 70 | 75 | 21 | 34 |
| 80 | 50 | 21 | 23 |
| 50 | 75 | 28 | 28 |
| 50 | 75 | 28 | NT* |
| 60 | 11 | 28 | 25 |
| 60 | 20 | 28 | 23 |

TABLE 12.2
APS behavior of drug product 2 (performed under the optimized
conditions as disclosed herein) under open dish storage
at various temperature humidity conditions

| | | | REI [ppm] |
|---|---|---|---|
| Temp. (° C.) | RH(%) | Time (days) | DP 2 |
| NA | NA | 0 | 16 |
| 80 | 50 | 7 | 18 |
| 50 | 75 | 14 | 21 |
| 60 | 50 | 14 | 18 |
| 60 | 75 | 14 | 22 |
| 70 | 31 | 14 | 17 |
| 70 | 75 | 14 | 23 |
| 50 | 75 | 21 | 20 |
| 60 | 11 | 21 | 19 |
| 60 | 75 | 21 | 21 |
| 70 | 50 | 21 | 16 |
| 70 | 75 | 21 | 23 |
| 80 | 50 | 21 | 17 |
| 50 | 75 | 28 | 19 |
| 50 | 75 | 28 | 19 |
| 60 | 11 | 28 | 18 |
| 60 | 50 | 28 | 18 |
| 60 | 75 | 28 | 22 |
| 60 | 75 | 28 | NT* |
| 70 | 31 | 28 | 19 |
| 70 | 50 | 28 | 17 |
| 70 | 75 | 28 | 27 |

Furthermore, the results for the registration stability of 3 batches as shown in the next table also do not exhibit any trend for the different packaging over time at different relative humidity and temperature.

TABLE 12.3
REI results in DP registration stability

| | | | Content of REI (ppm) | | |
|---|---|---|---|---|---|
| Timepoint | Storage condition | Packaging | (DP1) | (DP2) | (DP3) |
| Initial | | | 16 | 14 | 26 |
| 6 weeks | 5° C., ambient RH | Aluminum blister (PA/Al/PVC) | 15 | 14 | 26 |
| | | 40 mL HDPE Bottle, 30 counts | 17 | 16 | 29 |
| | | 40 mL HDPE Bottle, 60 counts | 16 | 16 | 28 |
| | 25° C., 05% RH | Aluminum blister | 15 | 14 | 26 |
| | | 40 mL HDPE Bottle, 30 counts | NT* | NT* | NT* |
| | | 40 mL HDPE Bottle, 60 counts | NT* | NT* | NT* |
| | 30° C., 75% RH | Aluminum blister (PA/Al/PVC) | 15 | 14 | 27 |
| | | 40 mL HDPE Bottle, 30 counts | 15 | 15 | 27 |
| | | 40 mL HDPE Bottle, 30 counts | 16 | 14 | 27 |
| | 5° C., Ambient RH | Aluminum blister | 15 | 14 | 27 |
| | | 40 mL HDPE Bottle, 30 counts | 16 | 15 | 26 |
| | | 40 mL HDPE Bottle, 60 counts | 15 | 15 | 27 |
| 3 months | 40° C., 75% RH | Aluminum blister (PA/Al/PVC) | 15 | 13 | 27 |
| | | 40 mL HDPE Bottle, 30 counts | 19 | 16 | 30 |
| | | 40 mL HDPE Bottle, 60 counts | 18 | 15 | 28 |
| | 30° C., 75% RH | Aluminum blister (PA/Al/PVC) | 15 | 13 | 26 |
| | | 40 mL HDPE Bottle, 30 counts | NT* | NT* | NT* |
| | | 40 mL HDPE Bottle, 60 counts | NT* | NT* | NT* |
| | 25° C., 60% RH | Aluminum blister (PA/Al/PVC) | 16 | 13 | 27 |
| | | 40 mL HDPE Bottle, 30 counts | 16 | 14 | 27 |
| | | 40 mL HDPE Bottle, 60 counts | 17 | 13 | 26 |
| | 5° C., Ambient RH | Aluminum blister (PA/Al/PVC) | 16 | 13 | 27 |
| | | 40 mL HDPE Bottle, 30 counts | 16 | 13 | 27 |
| | | 40 mL HDPE Bottle, 60 counts | 16 | 14 | 28 |
| | −20° C., Ambient | Aluminum blister (PA/Al/PVC) | 16 | 13 | 27 |
| | RH | 40 mL HDPE Bottle, 30 counts | 17 | 13 | 27 |
| | | 40 mL HDPE Bottle, 60 counts | 16 | 13 | 28 |

*RH: relative humidity, NT: not tested

Furthermore, the results for the registration stability of 1 batch for 18 and 24 months are shown in the next table. Longer stability study also does not exhibit any trend for the different packaging over time at different relative humidity and temperature.

TABLE 12.4
REI results in DP registration stability

| | | | Content |
|---|---|---|---|
| | Storage | | of REI |
| Timepoint | condition | Packaging | (ppm) |
| Initial | | | 21 |
| 18 months | 5° C., Ambient | Aluminum blister (PA/Al/PVC) | 25 |
| | RH | 40 mL HDPE Bottle, 30 counts | 25 |
| | | 40 mL HDPE Bottle, 60 counts | 23 |
| | 25° C., 60% RH | Aluminum blister | 26 |
| | | 40 mL HDPE Bottle, 30 counts | 24 |
| | | 40 mL HDPE Bottle, 60 counts | 26 |
| | 30° C., 75% RH | Aluminum blister (PA/Al/PVC) | 25 |
| | | 40 mL HDPE Bottle, 30 counts | NT |
| | | 40 mL HDPE Bottle, 60 counts | NT |
| 24 months | 5° C., Ambient | Aluminum blister (PA/Al/PVC) | 21 |
| | RH | 40 mL HDPE Bottle, 30 counts | 23 |
| | | 40 mL HDPE Bottle, 60 counts | 21 |
| | 25° C., 60% RH | Aluminum blister (PA/Al/PVC) | 22 |
| | | 40 mL HDPE Bottle, 30 counts | 23 |
| | | 40 mL HDPE Bottle, 60 counts | 23 |
| | 30° C., 75% RH | Aluminum blister (PA/Al/PVC) | 24 |
| | | 40 mL HDPE Bottle, 30 counts | NT |
| | | 40 mL HDPE Bottle, 60 counts | NT |

* NT: not tested

Conclusion: Within the variability of the analytical testing, REI does not develop further during stability, even at accelerated conditions, regardless the packaging configuration.

### Conclusion

Key findings from experiments conducted in Examples above and validation
Specific energy is the critical factor affecting drug substance particle size, with particle size decreasing as specific energy increases.
Specific energy is the critical factor impacting the formation of REI during wet media milling as REI levels rise with specific energy.
Suspension outlet temperature and average stress energy impact REI formation.
The study reveals that maintaining a specific energy range of 600 kJ/kg to 1000 kJ/kg ensure optimal product quality concerning particle size and impurity levels with a setpoint of 800 kJ/kg.
Average stress energy and suspension outlet temperature were investigated up to 1.32 μNm and 26° C. and resulted in REI content within the anticipated specification limits. However, a suspension outlet temperature of no more than 20° C. is preferred as it further decreases the REI content.
REI levels increase with high temperature (25° C. and 30° C.) over time in the intermediates and therefore storage of the wet media milling suspension and spray suspension at cooler temperatures is preferred to prevent further rise of REI content.
Excipient quality also impacts the quality of the drug product. Copovidone containing higher level of peroxide results in wet milling nanosuspension containing higher level of REI.
Level of REI do not increase during granulation, blending and tableting

### Example 13: Nanosuspension with Selected Scavenger (e.g. Antioxidants/Chelators)

The scavenging potential depends on the type of the scavenger used hence the type of scavenger can play a significant role in the reduction of epoxide (REI).

Since the amount of scavenger available for scavenging the peroxides and thus reducing the epoxide levels has a potential impact this was selected as one of the factors. Also, the scavenger levels, as for any functional excipient, was limited to avoid any unwanted risk (not above any FDA imposed acceptable level/maximum daily dose).

The presence of REI was evaluated in the planetary mill by adding different scavengers.

These experiments aimed to identify trends using small amounts of drug substance (10% w/w) for the selection of the best performing scavengers. The milling conditions are described in the table 13a below. While the results of the screening experiments are presented in Table 13b.

TABLE 13a
Milling Process in the planetary mill

| Milling | Size: | 1000 | μl |
|---|---|---|---|
| Chamber: | Material: | ZrO2 | |
| | 100% Grinding media charge Volume: | 1000.0 | μl |
| Grinding | Material: | ZrO2 | |
| media: | Diameter: | 200 | μm |
| | Bulk Density: | 3.58 | g/cm³ |
| | Amount: | 2148 | mg |
| | Bulk Volume: | 600.00 | μl |
| | Filling Level: | 60% | |
| Suspension | | ~620 | μl |
| volume: | | | |
| Mill: | Rotation speed: | 400 | rpm |

TABLE 13b
Experiment overview

| | | Scavenger | |
|---|---|---|---|
| Copovidone | | concentration | REI |
| supplier | Scavenger | [% w/w] | [ppm] |
| BASF | Cysteine | 0.02 | 41.1 |
| BASF | Lysine Hydrate | 0.02 | 45.7 |
| BASF | Methionine | 0.02 | 57.4 |
| BASF | Urea | 0.02 | 83.0 |
| BASF | Ascorbic Acid | 0.02 | 25.4 |
| BASF | Arginine | 0.02 | 61.3 |
| BASF | Tartaric Acid | 0.02 | 33.7 |
| BASF | VEDG-3.3 | 0.02 | 93.2 |
| BASF | Gentisic Acid | 0.02 | 18.4 |
| BASF | Gentisic Acid | 0.02 | — |
| BASF | Ascorbic Acid | 0.02 | — |
| BASF | Tartaric Acid | 0.02 | — |
| BASF | Benzyl alcohol | 0.05 | — |
| BASF | | | — |
| BASF | Vanillin | 0.01 | 29.0 |
| BASF | Thymol | 0.01 | 20.8 |
| BASF | Propyl gallate | 0.01 | 18.4 |
| BASF | EDTA | 0.01 | 31.4 |
| BASF | EDTA + Ascorbic | Each 0.01 | 15.4 |
| | acid | | |
| BASF | Mequinol | 0.02 | 10.7 |
| BASF | None (baseline) | 0.00 | 72.0 |
| BASF | None (platin) | 0.00 | 65.1 |
| BASF | Vanillin | 0.02 | 27.6 |
| | (stirred overnight) | | |
| BASF | Thymol | 0.02 | 21.3 |
| | (stirred overnight) | | |
| BASF | Vanillin | 0.02 | 27.5 |
| BASF | L-Methionine | 0.02 | 49.7 |
| BASF | BHT | | 44.0 |
| BASF | Sodium ascorbate | | 12.5 |
| BASF | Sodium | | 24.3 |
| | metabisulfite | | |
| BASF | ascorbate + citrate | | 6.6 |
| BASF | Ascorbate + | | 6.1 |
| | EDTA | | |
| BASF | Asc. Acid + citrate | | 7.9 |
| Ashland | Gentisic acid | | 18.1 |

It was shown that the peroxide content in the copovidone should be kept to low levels (e.g. less than 30 ppm, or less than 20 ppm and preferably less than 10 ppm)—Example 9a. Alternatively, or additionally, a scavenger (e.g. an antioxidant) can be added to better controlled the level of REI in wet media mill nanosuspension. In the instant experiment, the following scavengers were discarded due to lack of performance: VEDG-3.3, Urea, arginine, Methionine, BHT, lysine, cysteine.

Thymol and vanillin were not preferred due to their low vapor pressure. EDTA has a potential for generating nitrosamine impurities. Propyl gallate are big flakes and it has very low solubility in water. Therefore, Thymol, vanillin, EDTA and propyl gallate were not selected for further studies.

Mequinol performed well in reducing REI levels. It is commonly used in topical formulation. It is however an active ingredient and would need to be assessed with health authorities and therefore was also not selected for further studies.

Citrate buffer at 0.1M in combination with ascorbic acid or sodium ascorbate formed, a gel-like suspension.

Tartaric acid, despite its potential benefits, the screening experiments revealed that tartaric acid had limitations in performance for the intended application. Its interaction with other compounds during the experiments suggested potential issues with solubility and stability (blockage of the mill), which could affect the overall efficacy of the formulation.

Therefore, gentisic acid, sodium ascorbate and ascorbic acid were selected as best performing antioxidants.

### Example 14: Antioxidant Studies

Manufacturing batches were prepared according to Example 3, using the optimized process parameters for wet media milling process according to instant invention. The major difference of the process was the addition of an antioxidant in the raw suspension for milling as well as in the Matrix solution. The Matrix solution was then mixed with the suspension from the wet media milling resulting in the suspension for granulation. The addition of antioxidant was investigated to further minimize the formation of REI.

### Batch Tree for the Following Experiment

TABLE 14a
overview of technical batches manufactured

| Unit | | | | | | | | |
|---|---|---|---|---|---|---|---|---|
| Operations | Batch No. | | | | | | | |
| Batch ID Wet | LOU064- | LOU064- | LOU064- | LOU064- | LOU064- | LOU064- | PM1018 | PM1133 |
| media milling | munija 1-A- | brollu1- | brollu1- | munija1-A- | munija1-A- | brolllu1- | | |
| | 120 | 002 | 001 | 127 | 128 | 004 | | |
| Batch ID | LOU064- | LOU064- | LOU064- | LOU064- | LOU064- | LOU064- | PM1018 | PM1133 |
| Granulation | ORA-0258 | ORA- | ORA- | ORA-0278 | ORA-0279 | ORA-0285 | | |
| | | 0264 | 0265 | | | | | |
| Batch ID | LOU064- | LOU064- | LOU064- | LOU064- | LOU064- | LOU064- | PM1018 | PM1133 |
| Tabetting | ORA-0263 | ORA- | ORA- | ORA-0280 | ORA-0281 | ORA-0286 | | |
| | | 0270 | 0269 | | | | | |
| Batch ID | LOU064- | LOU064- | LOU064- | LOU064- | LOU064- | LOU064- | PM1018 | PM1133 |
| Filmcoating | ORA-0271 | ORA- | ORA- | ORA-0282 | ORA-0283 | ORA-0287 | | |
| | | 0275 | 0274 | | | | | |
| Additional | | | | | | | | |
| batch | | | | | | | | |
| information | | | | | | | | |
| Copovidone | Ashland | Ashland | Ashland | Ashland | Ashland | Ashland | Ashland | Ashland |
| supplier | | | | | | | | |
| Antioxidant | Reference | Gentisic | Gentisic | Ascorbic | Sodium | Sodium | — | Ascrobic |
| | batch | acid | acid | acid | Ascorbate | Ascorbate | | acid |
| Stabilizer | — | — | — | — | — | HPBC | — | — |
| Milling | Pendulum | pendulum | circulation | pendulum | pendulum | pendulum | pendulum | pendulum |
| opperation | | | | | | | | |

*HPBC: hydroxypropyl-beta-cyclodextrin

TABLE 14b
Wet media milling process overview

| | Batch | | | | | |
|---|---|---|---|---|---|---|
| | LOU064- | LOU064- | LOU064- | LOU064- | LOU064- | LOU064- |
| | muminja | brollu1- | brollu1- | muminja | munija | brollu1- |
| Process parameter | 1-A-120 | 001 | 002 | 1-A-127 | 1-A-128 | 004 |
| Operating mode | Pendulum | Circulation | Pendulum | Pendulum | Pendulum | Pendulum |
| Batch size/kg | 2 | 2 | 2 | 2 | 2 | 2 |
| Milling time/min | 85 | 184 | 93 | 56 | 59 | 44 |
| Passage number | 30 | n.a. | 30 | 15 | 15 | 15 |
| Turnover number | n.a. | 50 | n.a. | n.a. | n.a. | n.a. |
| (N)*/- | | | | | | |
| Suspension outlet | 16-26 | 19-23 | 17-24 | 14-23 | 18-22 | 17-20 |
| temperature (T) /° C. | | | | | | |
| (measured, range of | | | | | | |
| resulting parameter) | | | | | | |
| Rotor tip speed (vt)/ | 12.1-12.2 | 4.0-12.2 | 4.0-12.2 | 4.0-10.6 | 10.5-10.5 | 10.5-10.9 |
| m/s (calculated, | | | | | | |
| resulting parameter) | | | | | | |
| Cooling liquid inlet | 7-8 | 7-8 | 7-7 | 7-8 | 7-8 | 8-9 |
| temperature/° C. | | | | | | |

| Process parameter | PM1018 | PM1133 |
|---|---|---|
| Batch size/kg | 60 | 60 |
| Specific energy (E_{M})^{*,+}/kJ/kg | 764 | 752 |
| Average stress energy (S̅E̅) ^{#} μNm | 0.54-0.86 | 0.73-0.86 |
| Turnover number (N)^{*,+}/- | 55 | 58 |
| Passage number (N_{P})/- | 92 | 76 |
| Suspension outlet temperature (T)^{#}/° C. (measured, range of | 13-20 | 13-17 |
| resulting parameter) | | |
| Suspension inlet temperature (T_{S,in})/° C. (measured, range of | 10-21 | 10-21 |
| resulting parameter) | | |
| Rotor tip speed (vt)^{#}/m/s (calculated, resulting parameter) | 10.3-10.5 | 10.4-10.5 |
| Cooling liquid inlet temperature grinding chamber | 6-7 | 7-8 |
| (T_{C,in})/° C. (given parameter) | | |

TABLE 14c
antioxidant concentration

| | LOU064- | LOU064- | LOU064- | LOU064- | LOU064- | LOU064- | | |
|---|---|---|---|---|---|---|---|---|
| | ORA- | ORA- | ORA- | ORA- | ORA- | ORA- | | |
| Antioxidant | 0271 | 0283 | 0274 | 0275 | 0282 | 0287 | PM1018 | PM1133 |

Raw Suspension

| | | | | | | | | |
|---|---|---|---|---|---|---|---|---|
| Gentisic | — | — | 0.01% | 0.01% | — | — | — | — |
| acid | | | | | | | | |
| Ascorbic | — | — | — | — | 0.04% | — | — | 0.07 |
| acid | | | | | | | | |
| Sodium | — | 0.04% | — | — | — | 0.04% | — | — |
| Ascorbate | | | | | | | | |
| Matrix | | | | | | | — | — |
| solution | | | | | | | | |
| Gentisic | — | — | 0.08% | 0.08% | — | 0.27% | — | — |
| acid | | | | | | | | |
| Ascorbic | — | — | — | — | 0.27% | — | — | 0.16 |
| acid | | | | | | | | |
| Sodium | — | 0.27% | — | — | — | — | — | — |
| Ascorbate | | | | | | | | |

Spray suspension

| | | | | | | | | |
|---|---|---|---|---|---|---|---|---|
| Gentisic | — | — | 0.03% | 0.03% | — | — | — | — |
| acid | | | | | | | | |
| Ascorbic | — | — | — | — | 0.09% | — | — | — |
| acid | | | | | | | | |
| Sodium | — | 0.09% | — | — | — | 0.09% | — | — |
| Ascorbate | | | | | | | | |

The concentration of the Antioxidant was calculated as the mass of the Antioxidant divided by the sum of the mass of all compounds in the individual step.

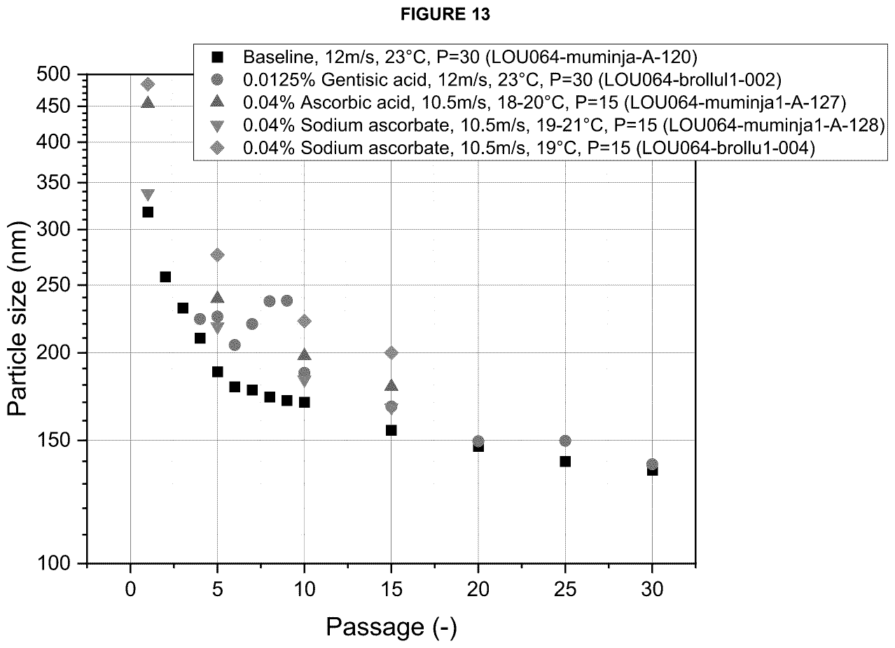

Particle sizes were analyzed for different formulation comprising antioxidant as compared to formulation without antioxidant and the results are depicted in FIG. 13. Analysis revealed that as the passage number increases, particle size (PS) typically decreases. some deviations were observed in experiments utilizing sodium ascorbate (specifically, brollul-004 and muminjal-A-128), which—despite being conducted with ostensibly identical process parameters-produced different PS values.

For the gentisic acid experiment, despite a non-explained anomaly (after six passages, PS surges unexpectedly, only to decrease again following ten passages) the particle size aligned with the baseline (i.e. no additional scavenger) by twenty passages. Not all batches were subjected to the same stirrer tip speeds, an operational variable that appears to subtly influence outcomes. Generally, higher tip speeds produced slightly reduced particle sizes at a given passage number, but intriguingly, muminja1-A-128 still achieves low PS at lower tip speed.

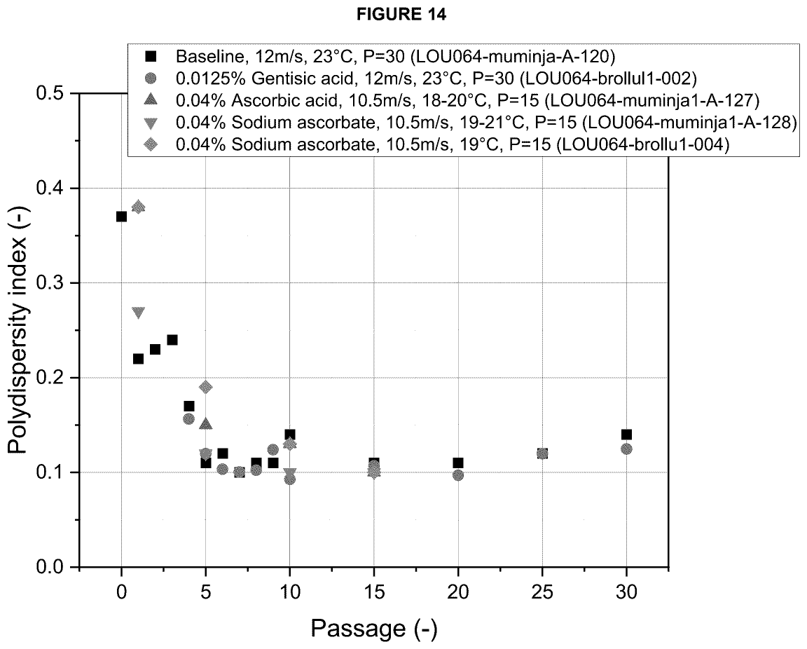

Polydispersity Indexes (PI) were determined for different formulations comprising antioxidant and the results were presented in FIG. 14. The relationship between polydispersity index (PI)-a measure of the width of the particle size distribution- and passage number remained consistent with the results obtained for formulation without antioxidant.

With increased passages, PI reliably decreased; after five passages, PI falls below 0.2 for all experiments, indicating that the resulting nanosuspensions exhibited an overall narrow particle size distribution. Notably, towards higher passage numbers (greater than 20), PI appeared to slightly increase again.

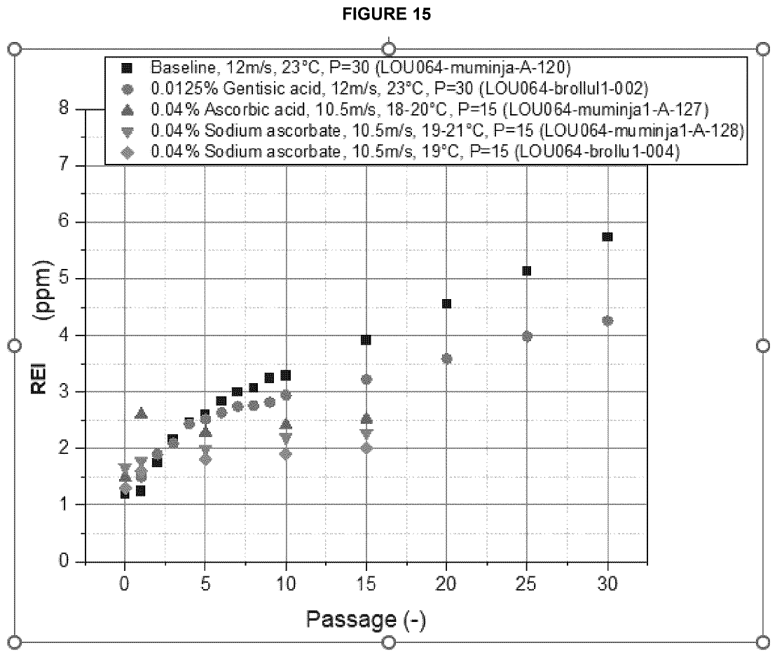

Examining the trends in REI impurity content as a function of passage number (FIG. 15), a general pattern emerged: increasing the passage number tended to result in a progressive rise in REI concentration. However, an exception to this trend was observed in the experiment utilizing ascorbic acid, where an initially sharp increase in REI content was followed by a plateau, with levels remaining relatively constant despite further passages.

When comparing experiments conducted at 12 m/s tip speed, the baseline experiment (no scavenger added) and the brollu1-002 batch containing gentisic acid exhibited divergent behaviors after five passages. Specifically, the presence of gentisic acid was associated with lower REI levels, indicating a potential mitigating effect of this scavenger on impurity formation during extended processing.

A separate comparison among experiments performed at 10.5 m/s tip speed revealed that the lowest overall REI concentrations at the end of the process are achieved in the batch containing sodium ascorbate.

### Example 15: Pendulum Operational Mode for Wet Media Milling Process

The impact of using antioxidants (described above) as well as the effect of changing the operating mode-specifically, comparing pendulum mode versus recirculation-on REI formation was investigated. These experiments were conducted both on laboratory scale (e.g. about 2 kg) and on pilot scale (e.g. 80 Kg) batches. In recirculation mode, the raw suspension was prepared in a vessel, pumped into the mill, and then recirculated within the same vessel.

This process continued until either the target specific energy (i.e. between about 600 and about 1000, preferably between about 600 and about 800 KJ/Kg) or the desired milling time was reached. In contrast, pendulum mode involved preparing the raw suspension in one vessel and then pumping it through the mill into a second vessel. Once the entire passage was completed, the suspension was pumped back from the second vessel through the mill into the first vessel.

The process was repeated until the defined number of passages or specific energy was achieved.

The main benefit of pendulum mode was that it reduces back-mixing of milled suspension with unmilled suspension, resulting in a narrower and more defined residence time distribution.

Operating modes are depicted in FIGS. 1a and 1b.

### Comparison Between Re-Circulation and Pendulum Operational Mode

REI formation vs Specific energy was measured in the 3 batches below:

### PM8440 Batch:

WMM with Re-circulation mode for 25 mg remibrutinib
25% LOU064
10.5 m/s tip speed
Copovidone (from Ashland)

### PM1018 Batch:

WMM with Pendulum mode for 100 mg remibrutinib
25% LOU064
Copovidone (from Ashland)
10.5 m/s tip speed

### PM1133 Batch:

WMM with Pendulum mode for 100 mg remibrutinib
25% LOU064
Copovidone (from Ashland)
10.5 m/s tip speed
Ascorbic acid

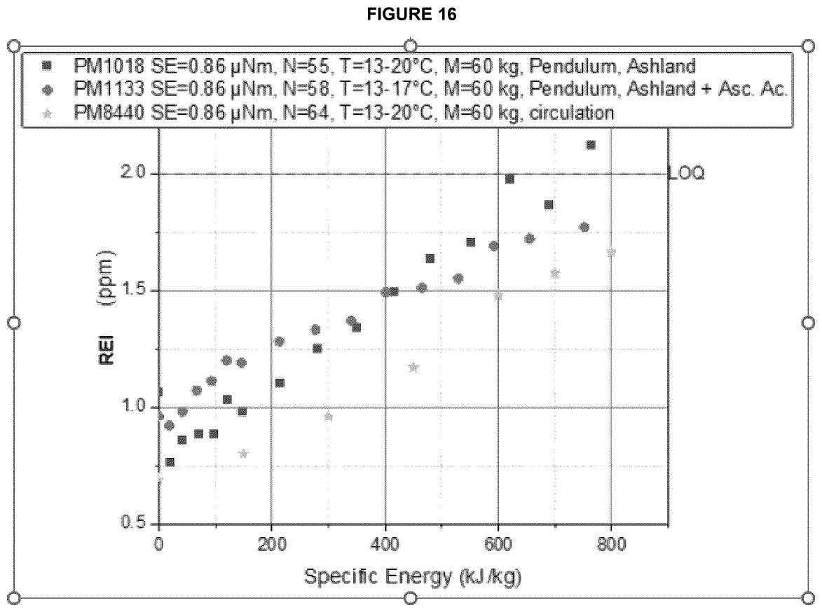

Results are presented in FIG. 16. FIG. 16 shows that REI values remained consistently below the analytical method's limit of quantification (LOQ) of 2 ppm throughout the milling process, with the sole exception of the end sample from the pendulum operating mode (PM1018). Examination of the raw data revealed that REI formation increases for both pendulum and circulation modes as specific energy rises, but almost all measured concentrations remained under the LOQ. There was a slight tendency for circulation mode to yield lower REI values than pendulum mode; however, given that all data points fall below the LOQ, these differences was not significant and did not indicate any clear advantage of one operating mode over the other.

### Summary of the Further Optimization of Milling Process:

The findings from the milling process analyses in Examples herein lead to several discoveries regarding REI formation and the effects of process parameters and operational modes.

Throughout all batches, the measured concentrations of REI remained consistently at or below the analytical method's limit of quantification (LOQ), set at 2 ppm. Even as specific energy was increased, the rise in REI was modest and differences between batches were relatively minor. A consistent benefit of adding an antioxidant—namely, a significant reduction in REI content—was not observed; but the batches with antioxidant displayed nevertheless a modest lower rate of increase.

Example 15 further examined the impact of switching the mill's operating mode from pendulum operation to circulation mode. Both operating modes produced highly consistent particle size profiles above 400 kJ/kg (and below 1000 KJ/Kg) specific energy. This consistency indicated that the transition between pendulum and circulation modes did not affect the overall efficiency of particle size reduction and indicated that specific energy was the principal parameter governing API particle size and REI content.

Polydispersity index (PI) data further reinforced this conclusion. For both pendulum and circulation operations, PI values were stable and low above 400 kJ/kg, indicating that particle distributions were highly uniform regardless of the selected milling configuration. The PI trends closely mirrored those seen in average particle size and together suggested that neither operating mode introduced significant variability into particle size distribution under the tested conditions.

Analysis of REI formation across operational modes revealed a similar pattern. Measured concentrations were below the LOQ for nearly all samples, except for the final sample from the pendulum-operated batch (PM1018). There was a mild tendency for circulation mode to yield slightly lower REI values than pendulum mode as specific energy increases, but since all data points remained below the LOQ, these differences were not statistically meaningful and offered no clear indication of an optimal mode for minimizing REI formation. Both operational modes can be equally used in the milling process.

Applicant hypothesized that pendulum operation might be advantageous for reducing REI formulation, due to a more narrowly defined residence time distribution and limited back-mixing. However, the process benefit could not be substantiated under current trial conditions (up to 60 kg). However, increasing batch size could differentiate both operation modes with respect to REI formation in a measurable way.

The Examples herein affirmed that process control and careful optimization of milling parameters as disclosed herein are essential for managing REI levels, with specific energy standing out as the most critical variable for both particle size determination and potential impurity control. Within the tested ranges and operational setups, antioxidant addition and pendulum operating mode were equally effective in limiting REI generation.

### Example 16: REI Evolution During Granulation, Sieving, Tableting and Coating

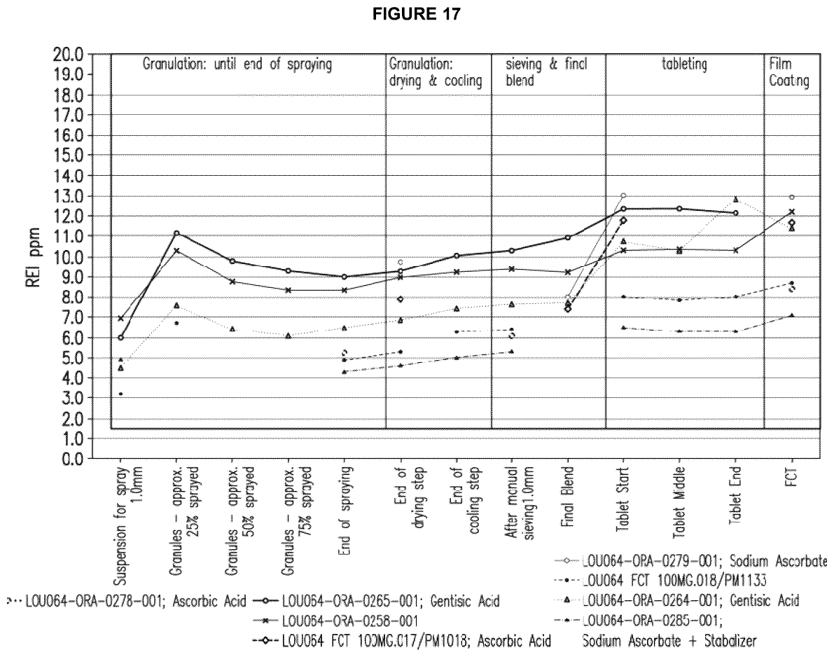

FIG. 17 represents the REI evolution during granulation, sieving, tableting and coating for different formulations

### Granulation

The granulation process started with preparing the matrix solution by adding purified water, polymer (Copovidone), and surfactant (Sodium Lauryl Sulfate). The grinded nanosuspension was then incorporated into the matrix solution for spray granulation. The process used top spray fluid bed granulation, where a carrier (Mannitol SD200) was pre-heated in the fluid bed. During the spray suspension application, the spray rate was gradually increased in ramp-up phases (e.g. start at about 200 g/kg-300 g/kg for commercial scale) to reach the final spray rate at steady state (e.g. between 500 and 600 g/min for commercial scale). The atomization air pressure was typically between 2 to 3.5 bar (e.g. 3 bar for commercial scale).

The spray granulation parameters (spray rate and atomization pressure) determined the quality of the granules. Coarse granules were generally the outcome of a high process moisture (lower atomization air pressure/high spray rate), while fine granules were generally the outcome of a dryer process (high atomization air pressure and lower spray rate).

The influence of antioxidants on granule size was preliminary assessed. Ascorbic Acid did not significantly affect granule size. Gentisic Acid resulted in slightly larger granules compared to the reference batch, but the impact was not significant.

Parameters (atomization air pressure and spray rate) can be adjusted accordingly by the skilled person in the art, depending on the process scale (laboratory scale (e.g. about 2 Kg), pilot scale (e.g. about 80 kg) or commercial scale (e.g. about 200 kg)) in order to obtain the granules with desired attributes (particle size distribution, bulk density, tapped density.) With respect to the REI content during granulation process, FIG. 17 shows that in general, the REI level was increasing at the beginning of the granulation process by approximately 3 ppm to 6 ppm (difference between REI concentration at 25% suspension sprayed to REI concentration in the suspension to spray). From 25% suspension sprayed onwards to 75% the REI level was decreasing. The reduction in the REI concentration was caused by the fact that the REI concentration in the “suspension to spray” was lower than the REI concentration in the granulate at 25% suspension sprayed. This correlation can also be demonstrated through a dilution calculation. This observation suggested that the granule's carrier particle played a role in the formation of REI. As the granulation process approached completion (after 75% of the suspension has been sprayed), there was a slight increase in the REI concentration within the granule. There was no evidence that the scavenger type had an impact on this small increase during granulation step.

Despite a slight increase of REI content during the granulation step, the REI content remained below 15 ppm, e.g. less than 12 ppm, less than 10 ppm or less than 7 ppm.

### Final Blend

The blending process involved combining screened granules with excipients such as Mannitol SD200, Cellulose MK GR or Microcrystalline Cellulose PH102, Crosscarmellose Sodium, and Sodium Stearyl Fumarate, followed by sieving and re-blending to achieve a uniform final blend. Particle size distribution (PSD) of the final blend trends mirrored those of the granules.

The blend was sieved through a 1 mm sieve, and the sieved material was blended again to get the final blend. FIG. 17 shows that the REI concentration can be taken to be constant in the blending process. The sieving & blending process, therefore, had no significant contribution to the REI formation.

### Tableting

During compression of the tablet, there was another small increase of the REI concentration. But the compression force had no impact on the REI formation. No significant differences were observed between minimum or maximum hardness. (Table 17)

| | Compression | | |
|---|---|---|---|
| Final blend | force (kN) | Hardness [N] | REI [ppm] |
| 11.28 mm round flat faced | 5 | 23 | 11 |
| Embossing: none | 40 | 208 | 12 |

The compression force applied is to be adjusted to preferably reach a hardness between 120 and 200 [N].

For Middle and End of the tableting process, the REI concentration stayed constant as highlighted in FIG. 17.

### Coating

A typical process for the coating process was as follows: At the start of phase 1 the machine was heated up without cores. In Phase 2, the cores were added to the pan coater and heated up. In Phase 3, the spray phase, the spray rate was ramped up at the beginning (phase 3.1) until the steady state phase is reached in phase 3.2 (steady state phase is indicated by the pump speed). At the end of this spraying phase, the film coated tablet was dried (phase 4) and cooled down in phase 5.

Tablets were coated with an OPADRY II PVA-based formulation containing red and black pigments dispersed in demineralized water. Key process parameters included a spray rate of 3.0-3.5 g/min, constant atomization and pattern pressure at 0.7 bar, and an inlet air temperature of 80° C. Outlet air temperature ranged between 42.7° C. and 44.2° C. across batches.

Tableting and Film coating processes did not contribute or increase the REI content for the final drug product.

Despite a small increase of REI content at the granulation and the compression processes, FIG. 17 shows that the film coated tablet obtained by instant invention (optimized wet media milling process) had a content of REI below 15 ppm, e.g. below 12 ppm, e.g. below 10 ppm, e.g. below 7 ppm.

### Example 17. 100 mg Film Coated Tablet (Ascorbic Acid as Antioxidant)

Using the optimized parameters of this invention, the following film coated tablet formulation were prepared.

### Manufacturing Process

Preparation of suspension for granulation
   Preparation of Tablet Core and Coating

### Detailed Process Flow Description for Wet Media Mill

The process was operated in pendulum mode. Specifically, the suspension was pumped from the preparation vessel (vessel 1) through the mill into a receiving vessel (vessel 2). Upon completion of the initial passage into the receiving vessel, the suspension was recirculated back into vessel 1 via the mill. This procedure was repeated until the target specific energy was achieved.
Specific energy: 800 kJ/Kg)
Rotor speed: 912 rpm
Rotor tip speed: 10.5 m/s
Average stress energy: 1.3 pNm
Number of passages/turnover number: 106
Suspension outlet temperature: 20° C.

### Composition

| | 1batch = 150′000 units per batch | | |
|---|---|---|---|
| | Formula per | Formula per | Batch |
| LOU064 FCT 100 MG | unit | unit | size |
| Component name | [mg/unit] | [% w/w] | [kg/batch] |

Raw suspension

| | | | |
|---|---|---|---|
| LOU064 | 100.000 | 19.231 | 15.000 |
| Plasdone S-630 Ultra | 16.000 | 3.077 | 2.400 |
| Copovidone | | | |
| Sodium Lauryl Sulfate | 0.400 | 0.077 | 0.060 |
| Ascrobic acid | 0.08 | 0.015 | 0.012 |
| Purified water | 283.520 | 54.523 | 42.528 |
| | | | 60.000 |

Matrix solution

| Plasdone S-630 Ultra | 34.000 | 6.538 | 5.100 |
|---|---|---|---|
| Copovidone | | | |
| Sodium Lauryl Sulfate | 3.600 | 0.692 | 0.540 |
| Ascorbic acid | 0.170 | 0.033 | 0.026 |
| Purified water | 88.546 | 17.028 | 13.282 |
| Suspension for spray granulation amount | | | 78.947 |
| Mannitol SD200 | 157.750 | 30.337 | 23.663 |
| Granulate amount | | | 46.800 |
| Microcrystalline cellulose | 85.800 | 16.500 | 12.870 |
| PH102 [Cellulose MK GR] | | | |
| Mannitol SD 200 | 85.800 | 16.500 | 12.870 |
| Croscarmellose sodium | 31.200 | 6.000 | 4.680 |
| [Natrium-CMC-XL] | | | |
| Sodium Stearyl Fumarate | 5.200 | 1.000 | 0.780 |
| Core tablet weight | 520.000 | 100.000 | 78.000 |
| OPADRY II WHITE | 22.975 | 4.418 | 3.446 |
| 85F48105.001 | | | |
| OPADRY II RED | 0.879 | 0.169 | 0.132 |
| 85F250061.001 | | | |
| OPADRY II BLACK | 0.147 | 0.028 | 0.022 |
| 85F277001.001 | | | |
| Purified water | 137.133 | 26.372 | 20.57 |
| Film coated tablet | 544.000 | 104.615 | 81.600 |

### Example 18. 100 mg Film Coated Tablet (Gentisic Acid as Antioxidant)

Using the optimized parameters of this invention, the following film coated tablet formulation were prepared.

Manufacturing process
Preparation of suspension for granulation
   Preparation of Tablet Core and Coating

### Detailed Process Flow Description for Wet Media Mill

The process was operated in pendulum mode. Specifically, the suspension was pumped from the preparation vessel (vessel 1) through the mill into a receiving vessel (vessel 2). Upon completion of the initial passage into the receiving vessel, the suspension was recirculated back into vessel 1 via the mill. This procedure was repeated until the target specific energy was achieved.
Specific energy: 800 kJ/Kg)
Rotor speed: 912 rpm
Rotor tip speed: 10.5 m/s
Average stress energy: 1.3 pNm
Number of passages/turnover number: 106
Suspension outlet temperature: 20° C.

### Composition

| | 1batch = 150′000 units per batch | | |
|---|---|---|---|
| | Formula per | Formula per | Batch |
| LOU064 FCT 100 MG | unit | unit | size |
| Component name | [mg/unit] | [% w/w] | [kg/batch] |

Raw suspension

| | | | |
|---|---|---|---|
| LOU064 | 100.000 | 19.231 | 15.000 |
| Plasdone S-630 Ultra | 16.000 | 3.077 | 2.400 |
| Copovidone | | | |
| Sodium Lauryl Sulfate | 0.400 | 0.077 | 0.060 |
| Gentisic acid | 0.050 | 0.010 | 0.008 |
| Purified water | 283.550 | 54.529 | 42.533 |
| | | | 60.000 |

Matrix solution

| Plasdone S-630 Ultra | 34.000 | 6.538 | 5.100 |
|---|---|---|---|
| Copovidone | | | |
| Sodium Lauryl Sulfate | 3.600 | 0.692 | 0.540 |
| Gentisic acid | 0.106 | 0.020 | 0.016 |
| Purified water | 88.610 | 17.040 | 13.291 |
| Suspension for spray granulation amount | | | 78.947 |
| Mannitol SD200 | 157.844 | 30.355 | 23.677 |
| Granulate amount | | | 46.800 |
| Microcrystalline cellulose | 85.800 | 16.500 | 12.870 |
| PH102 [Cellulose MK GR] | | | |
| Mannitol SD 200 | 85.800 | 16.500 | 12.870 |
| Croscarmellose sodium | 31.200 | 6.000 | 4.680 |
| [Natrium-CMC-XL] | | | |
| Sodium Stearyl Fumarate | 5.200 | 1.000 | 0.780 |
| Core tablet weight | 520.000 | 100.000 | 78.000 |
| OPADRY II WHITE | 22.975 | 4.418 | 3.446 |
| 85F48105.001 | | | |
| OPADRY II RED | 0.879 | 0.169 | 0.132 |
| 85F250061.001 | | | |
| OPADRY II BLACK | 0.147 | 0.028 | 0.022 |
| 85F277001.001 | | | |
| Purified water | 137.133 | 26.372 | 20.57 |
| Film coated tablet | 544.000 | 104.615 | 81.600 |

### Example 19. 100 mg Film Coated Tablet (Sodium Ascorbate as Antioxidant)

Using the optimized parameters of this invention, the following film coated tablet formulation was prepared.

Manufacturing process and detailed flow description for wet media milling is as disclosed in Example 18.

### Composition

| | 1batch = 150′000 units per batch | | |
|---|---|---|---|
| | Formula per | Formula per | Batch |
| LOU064 FCT 100 MG | unit | unit | size |
| Component name | [mg/unit] | [% w/w] | [kg/batch] |

Raw suspension

| | | | |
|---|---|---|---|
| LOU064 | 100.000 | 19.231 | 15.000 |
| Plasdone S-630 Ultra | 16.000 | 3.077 | 2.400 |
| Copovidone | | | |
| Sodium Lauryl Sulfate | 0.400 | 0.077 | 0.060 |
| Sodium ascorbate. | 0.160 | 0.031 | 0.024 |
| Purified water | 283.440 | 54.508 | 42.516 |
| | | | 60.000 |

Matrix solution

| Plasdone S-630 Ultra | 34.000 | 6.538 | 5.100 |
|---|---|---|---|
| Copovidone | | | |
| Sodium Lauryl Sulfate | 3.600 | 0.692 | 0.540 |
| Sodium ascorbate | 0.340 | 0.065 | 0.051 |
| Purified water | 88.376 | 16.995 | 13.256 |
| Suspension for spray granulation amount | | | 78.947 |
| Mannitol SD200 | 157.500 | 30.288 | 23.625 |
| Granulate amount | | | 46.800 |
| Microcrystalline cellulose | 85.800 | 16.500 | 12.870 |
| PH102 [Cellulose MK GR] | | | |
| Mannitol SD 200 | 85.800 | 16.500 | 12.870 |
| Croscarmellose sodium | 31.200 | 6.000 | 4.680 |
| [Natrium-CMC-XL] | | | |
| Sodium Stearyl Fumarate | 5.200 | 1.000 | 0.780 |
| Core tablet weight | 520.000 | 100.000 | 78.000 |
| OPADRY II WHITE | 22.975 | 4.418 | 3.446 |
| 85F48105.001 | | | |
| OPADRY II RED | 0.879 | 0.169 | 0.132 |
| 85F250061.001 | | | |
| OPADRY II BLACK | 0.147 | 0.028 | 0.022 |
| 85F277001.001 | | | |
| Purified water | 137.133 | 26.372 | 20.57 |
| Film coated tablet | 544.000 | 104.615 | 81.600 |

### CONCLUSION

The examples 13-19 described herein provide a comprehensive analysis of the technical development and pilot scale batches of LOU064 Film-Coated Tablets (FCT) (e.g. 25 mg, 100 mg). The objective was to evaluate the impact of various antioxidants and a stabilizer on the formation of REI, a critical impurity, during the manufacturing process and the processability.

The study focused on wet media milling, fluid bed granulation, tableting, and coating processes. The formulation was modified by adding antioxidants such as gentisic acid, ascorbic acid, sodium ascorbate and a stabilizer (Hydroxypropyl-beta-Cyclodextrin) to prevent or reduce the REI formation. For all batches produced, the REI concentration in the Film Coated Tablet (FCT) is below 50 ppm, below 30 ppm, below 15 ppm, or below 7 ppm.

The milling study indicated that REI formation remained negligible when using optimized wet media milling parameters according to the invention; concentrations of REI remained consistently at or below the analytical limit of quantification (2 ppm). Antioxidant addition provided only small benefit in reducing the level of REI.

Operational mode comparisons (pendulum vs. circulation) revealed no meaningful impact on particle size reduction or impurity formation. Both modes delivered consistent particle size profiles and low polydispersity index (PI) values above 400 kJ/kg, validating the mechanistic model that specific energy was the primary driver of particle size and REI formation.

Overall, findings emphasize that specific energy was the critical parameter for particle size control and impurity management, while operating mode offered no substantial advantage under current conditions.

Experiments have shown a small increase of REI content during the granulation and the compression processes, but the optimized wet media milling parameters of instant invention (specific energy, average stress energy, suspension outlet temperature, optionally in the presence of antioxidant and/or using a pendulum operational mode) have allowed the preparation of pharmaceutical compositions (e.g. film coated tablets, e.g. 25 mg, 100 mg of remibrutinib) with a content of REI below 15 ppm, e.g. below 12 ppm, e.g. below 10 ppm, e.g. below 7 ppm.

1. A composition comprising or consisting of remibrutinib, or a pharmaceutically acceptable salt of remibrutinib, wherein said composition is substantially free of the epoxide impurity of Formula:

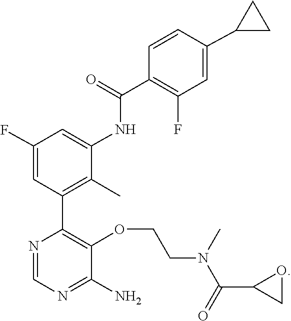 [SMILES: Cc1c(NC(=O)c2ccc(C3CC3)cc2F)cc(F)cc1-c1ncnc(N)c1OCCN(C)C(=O)C1CO1]

2. A composition of claim 1 comprising or consisting of remibrutinib or a pharmaceutically acceptable salt thereof, wherein the total amount of epoxide impurity in the composition is no more than about 75 ppm, e.g. no more than about 50 ppm, e.g. no more than about 30 ppm; or e.g. no more than about 7.5 ppm, relative to the total amount of remibrutinib in free or salt form.

3-5. (canceled)

6. A composition according to claim 1, further comprising one or more pharmaceutically acceptable excipients.

7. A composition according to claim 6 wherein one or more pharmaceutically acceptable excipients are selected from surfactant, binder, filler, disintegrant, lubricant and antioxidant.

8-11. (canceled)

12. The composition of claim 1, wherein the total amount of remibrutinib or a pharmaceutically acceptable salt thereof in the composition provides the same amount of remibrutinib as 10 mg, 25 mg or 100 mg of remibrutinib free base.

13-15. (canceled)

16. A method of preparing a composition comprising remibrutinib or a pharmaceutically acceptable salt of remibrutinib and one or more pharmaceutically acceptable excipients, wherein the method comprises a step of particle size reduction of remibrutinib by a wet media milling process wherein the wet media milling process is performed with a specific energy between 600 and 1000 KJ/Kg, preferably between 600 and 800 KJ/Kg.

17. A method of preparing a composition comprising remibrutinib or a pharmaceutically acceptable salt of remibrutinib and one or more pharmaceutically acceptable excipients according to claim 16, wherein the wet media milling process is performed at an average stress energy process parameter of no more than 1.3 μNm.

18. The method of preparing a composition comprising remibrutinib or a pharmaceutically acceptable salt of remibrutinib and one or more pharmaceutically acceptable excipients according to claim 16, wherein the wet media milling process is performed in grinding chamber with suspension outlet temperature of no more than 20° C.

19. The method of preparing a composition comprising i) remibrutinib or a pharmaceutically acceptable salt of remibrutinib and ii) one or more pharmaceutically acceptable excipients according to claim 16, wherein the wet media milling process is performed in recirculation mode with a turnover number of between about 28 to about 106.

20. The method of preparing a composition comprising i) remibrutinib or a pharmaceutically acceptable salt of remibrutinib and ii) one or more pharmaceutically acceptable excipients according to claim 16, wherein the wet media milling process generates a wet media milled suspension with a particle size of remibrutinib of less or equal to 200 nm.

21-23. (canceled)

24. The method of preparing a composition comprising i) remibrutinib or a pharmaceutically acceptable salt of remibrutinib and ii) one or more pharmaceutically acceptable excipients according to claim 16, further comprising additional steps of preparing suspension for a spray granulation step.

25-26. (canceled)

27. The method of claim 20 or 24, wherein the media milled suspension and the suspension for spray granulation comprises one or more pharmaceutically acceptable excipients selected from a surfactant and a binder.

28. The method of claim 27, wherein the media milled suspension and the suspension for spray granulation comprises a surfactant selected from sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof, preferably the surfactant is sodium lauryl sulfate.

29. The method of claim 27 wherein the media milled suspension and the suspension of spray granulation comprises a binder selected from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, vitamin E TPGS, or a mixture thereof, preferably the binder is polyvinylpyrrolidone-vinyl acetate copolymer (copovidone).

30. The method of claim 24, further comprising the step of spray granulation wherein the suspension for spray granulation is sprayed onto an inert substrate to provide a granule.

31-32. (canceled)

33. The method of claim 30 wherein the inert substrate is selected from the group consisting of lactose, microcrystalline cellulose, mannitol, sucrose, starch, granulated hydrophilic fumed silica, or mixtures thereof, preferably the material, which is selected from the group consisting of lactose, mannitol, or mixtures thereof and most preferably the material is mannitol.

34. The method of claim 30 to further comprising the step of preparing a final blend.

35. The method of claim 34, wherein the final blend comprises one or more pharmaceutically acceptable excipient is selected from a filler, a disintegrating agent, a lubricating agent, and a gliding agent.

36. The method of claim 35, wherein the filler is selected from calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof preferably mannitol or cellulose or mixture thereof.

37. The method of claim 35 wherein the disintegrating agent is selected from croscarmellose sodium, crospovidone, sodium starch glycolate, corn starch, or alginic acid, or mixture thereof.

38. The method of claim 35 wherein the lubricating agent is selected from magnesium stearate, sodium stearyl fumarate, stearic acid or talc or mixture thereof.

39. The method according to claim 35, further comprising step of compressing the final blend into orally administrable dosage form, optionally a tablet.

40. The method according to claim 35 wherein the pharmaceutically acceptable excipients have a peroxide content of less than 30 ppm, less than 20 ppm or less than 10 ppm, e.g. copovidone as a binder has a peroxide content of less than 30 ppm, less than 20 ppm or less than 10 ppm.

41-87. (canceled)

88. A method of treating a disease or a disorder mediated by BTK or ameliorated by inhibition of BTK, in a patient in need of such treatment, comprising administering a composition according to claim 1, wherein the disease or disorder is selected from an autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis especially relapsing multiple sclerosis, myasthenia gravis, Sjogren's syndrome, hidradenitis suppurativa, IgE driven allergy, e.g. drug, venom, food allergy; autoimmune hemolytic anemia, anti-neutrophil cytoplasmic antibodies (ANCA)-associated vasculitis, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerulonephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of hematopoietic origin including, but not limited to, multiple myeloma; a leukemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenström disease.

89. (canceled)

90. The method of claim 88 wherein the treatment comprises administering to the patient a daily amount of remibrutinib of 50 mg, or a daily amount of the pharmaceutically acceptable salt of remibrutinib which corresponds to 50 mg of remibrutinib free base.

91. The method of claim 89, wherein the treatment comprises administering to the patient a daily amount of remibrutinib of 200 mg, or a daily amount of the pharmaceutically acceptable salt of remibrutinib which corresponds to 200 mg of remibrutinib free base.

92. The method according to claim 88 wherein the patient to be treated receives a daily amount of epoxide, e.g. remibrutinib epoxide impurity (REI) of no more than 1.5 ug.

93-94. (canceled)
